(19) United States
(12) Patent Application Publication
(10) Pub. No.: US 2025/0049660 A1
(43) Pub. Date: Feb. 13, 2025

(54) ENOL ETHER PROPERFUME

(71) Applicant: FIRMENICH SA, Satigny (CH)

(72) Inventor: Gary Bernard WOMACK, Plainsboro, NJ (US)

(73) Assignee: FIRMENICH SA, Satigny (CH)

(21) Appl. No.: 18/719,325

(22) PCT Filed: Dec. 14, 2022
(86) PCT No.: PCT/EP2022/085830

§ 371 Date: Jun. 13, 2024

Related U.S. Application Data
(60) Provisional application No. 63/289,339, filed on Dec. 14, 2021.

(30) Foreign Application Priority Data
Jan. 13, 2022 (EP) 22151258.5

(51) Int. Cl. A61K 8/33; A61Q 13/00; C07C 43/16

(52) U.S. Cl. CPC A61K 8/33; A61Q 13/00; C07C 43/16; A61K 2800/57

(57) ABSTRACT

The present invention relates to enol ether compounds of formula (I) as properfume compounds. In particular, the present invention relates to a method to release a compound being an aldehyde compound of formula (II), a formate ester of formula (III) and/or an alcohol of formula (IV), by exposing the enol ether compound of formula (I) to an environment wherein it is oxidized. Moreover, the present invention relates to a perfuming composition and a perfumed consumer product comprising at least one enol ether compound of formula (I).

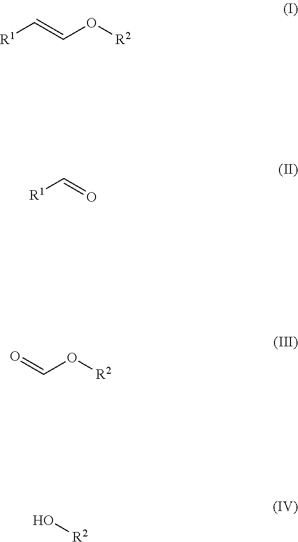

### TECHNICAL FIELD

The present invention relates to compounds of formula (I) as properfume compounds. In particular, the present invention relates to a method to release a compound being an aldehyde compound of formula (II), a formate ester of formula (III) and/or an alcohol of formula (IV), by exposing the compound of formula (I) to an environment wherein it is oxidized. Moreover, the present invention relates to a perfuming composition and a perfumed consumer product comprising at least one compound of formula (I).

### BACKGROUND

The perfume industry has a particular interest for compositions or additives which are capable of prolonging or enhancing the perfuming effect of at least one perfuming ingredient for a certain period of time. It is particularly desirable to obtain long-lasting properties for standard perfumery raw materials which are too volatile or have a poor substantivity by themselves, or which are only deposited in a small amount onto the surface of the final application. Furthermore, some of the perfumery ingredients are unstable and need to be protected against slow degradation prior to their use. Long-lasting perfumes are desirable for various applications, as for example fine or functional perfumery or cosmetic preparations. The washing and softening of textiles are particular fields in which there is a constant need to enable the effect of active substances, in particular perfumes, or perfuming compositions, to be effective for a certain period of time after washing, softening and drying. Indeed, many active substances which are particularly suitable for this type of application are known to lack tenacity on laundry, or do not remain on the laundry when rinsed, with the result that their perfuming effect is experienced only briefly and not very intensely. Given the importance of this type of application in the perfume industry, research in this field has been sustained, in particular with the aim of finding new, and more effective solutions to the aforementioned problems.

WO 2019243501 discloses enol ether capable of efficiently releasing an aryl aldehyde, a formate ester compound and an alcohol compound. However, said enol ethers do not allow releasing alkyl aldehyde as the access to this kind of enol ether is very challenging.

It has now been surprisingly found that enol ether compounds according to the present invention may be prepared efficiently allowing to release alkyl aldehyde compound of formula (II) while still being able of efficiently releasing a compound being a formate ester of formula (III) and/or an alcohol of formula (IV).

### DETAILED DESCRIPTION

Olfaction is a complex and dynamic process and controlling the release profile of volatile fragrance compounds may maximize the impact of fragrance formulations and enrich the sensorial experience. Profragrances, such as the compounds of the present invention add a dimension of control and long-lastingness to the release profile of highly volatile perfumery raw materials (PRMs), such as alkyl aldehyde highly sought and representing an important group of compounds in perfumery field.

Without intending to be limited to any particular theory, the compounds of the present invention may achieve their effect on the olfactive properties of a perfuming composition by tethering the PRM to a molecular anchor and requiring a specific reaction mechanism under certain environmental conditions to release the volatile PRM from this anchor. In the present invention, the release of one, two or up to three PRMs is prompted by oxidation when the profragrance is exposed to the oxygen in ambient air.

Herein disclosed, a method to release from a precursor compound, compounds selected from the group consisting of
a) an aldehyde compound of formula

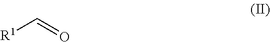

wherein
R¹ is a C₁₋₁₅ alkyl, C₃₋₁₅ alkenyl, C₃₋₁₅ cycloalkyl, C₅₋₁₅ cycloalkenyl or C₃₋₁₄ heterocycloalkyl group, each optionally substituted with one or more of a hydroxy, C₁₋₁₅ alkyl, C₂₋₁₅ alkenyl, C₁₋₁₅ alkoxy, C₂₋₁₅ alkenyloxy, C₃₋₁₅ cycloalkyl, C₅₋₁₅ cycloalkenyl, C₃₋₁₅ heterocycloalkyl, carboxylic acid, C₁₋₄ carboxylic ester, C₆₋₁₀ aryl and/or C₆₋₁₀ aryloxy group, each optionally substituted with one or more of a C₁₋₈ alkyl, C₁₋₈ alkoxy, hydroxy, carboxylic acid and/or C₁₋₄ carboxylic ester group;
b) a formate ester of formula

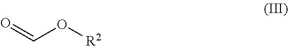

R² is a C₁₋₁₈ hydrocarbon group optionally comprising one, two or three oxygen atoms; provided that an ester functional group alpha to the formyloxy group is excluded;
c) an alcohol of formula

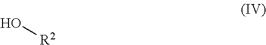

wherein R² has the same meaning as defined above;
wherein the precursor compound comprises a compound of formula (I)

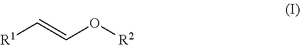

in the form of any one of its stereoisomers or a mixture thereof, and wherein R¹ and R² have the same meaning as defined above;
   by exposing the precursor compound of formula (I) to an environment wherein the compound is oxidized.

The first object of the present invention is a method to release from a precursor compound, compounds selected from the group consisting of
a) an aldehyde compound of formula

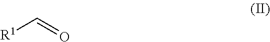

wherein
R¹ is a C₁₋₁₅ alkyl, C₃₋₁₅ alkenyl, C₃₋₁₅ cycloalkyl, C₅₋₁₅ cycloalkenyl or C₃₋₁₄ heterocycloalkyl group, each optionally substituted with one or more of a hydroxy, C₁₋₁₅ alkyl, C₂₋₁₅ alkenyl, C₁₋₁₅ alkoxy, C₂₋₁₅ alkenyloxy, C₃₋₁₅ cycloalkyl, C₅₋₁₅ cycloalkenyl, C₃₋₁₅ heterocycloalkyl, carboxylic acid, C₁₋₄ carboxylic ester, C₆₋₁₀ aryl and/or C₆₋₁₀ aryloxy group, each optionally substituted with one or more of a C₁₋₈ alkyl, C₁₋₈ alkoxy, hydroxy, carboxylic acid and/or C₁₋₄ carboxylic ester group;
b) a formate ester of formula

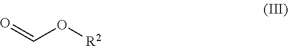

R² is a C₄₋₈ hydrocarbon group optionally comprising one or two oxygen atoms; provided that an ester functional group alpha to the formyloxy group is excluded and provided that R² does not comprise an allylic functional group;
c) an alcohol of formula

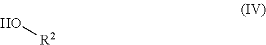

wherein R² has the same meaning as defined above;
wherein the precursor compound comprises a compound of formula (I)

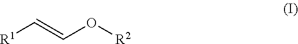

in the form of any one of its stereoisomers or a mixture thereof, and wherein R¹ and R² have the same meaning as defined above;
   by exposing the precursor compound of formula (I) to an environment wherein the compound is oxidized.

According to any one of the embodiments of the invention, at least one of the compounds of formula (II), (III) or (IV) is an active compound.

The terms “active compound”, “active volatile compound”, “active volatile aldehyde, formate ester and/or alcohol” or the similar, are understood as aldehyde, formate ester and/or alcohol compounds being capable of bringing a benefit or effect into its surrounding environment. In particular, the “active compound” is selected from the group consisting of a perfuming ingredient, flavoring ingredient, malodor counteracting ingredient, antimicrobial ingredient and insect repellent or attractant ingredient. Therefore, to be considered as an “active compound” the compound has to possess at least one property which renders it useful as a perfuming ingredient, as a malodor counteracting ingredient, as a flavoring ingredient, as an antimicrobial ingredient and/or as an insect repellent or attractant.

The term “perfuming ingredient” is understood as a compound which is used as an active ingredient in perfuming preparations or compositions in order to impart a hedonic effect. In other words, a compound to be considered as being a perfuming ingredient, must be recognized by a skilled person in the art of perfumery as being able to impart or modify in a positive or pleasant way the odor of a composition, and not just as having an odor. The perfuming ingredient may impart an additional benefit beyond that of modifying or imparting an odor, such as long-lasting, blooming, malodour counteraction, antimicrobial effect, antiviral effect, microbial stability, or pest control. The term “flavoring ingredient” is understood to as being capable of imparting a taste sensation to the taster's pallet. The term “malodor counteracting ingredient” is understood as being capable of reducing the perception of malodor, i.e. of an odor that is unpleasant or offensive to the human nose. The term “antimicrobial ingredient” is understood as being capable of killing microorganism or reducing or preventing their growth and/or accumulation and include antibacterial, antibiotic, antifungal, antiviral and antiparasitic ingredients. The term “insect attractant or repellent” is understood as a compound having a positive or negative effect on insects. Examples of insect attractant or repellent ingredients can be found in reference texts or in other works of a similar nature as for example: A. M. El-Sayed, The Pherobase 2005, http://www.pherobase.net.

According to the above and below mentioned embodiments of the invention, the method according to the present invention is particularly useful when the active compound is a perfuming ingredient, i.e. a perfuming aldehyde compound, formate ester and/or alcohol. A “perfuming aldehyde compound, formate ester and/or alcohol” is a compound, which is of use in the perfumery industry, i.e. a compound which is used as active ingredient in perfuming preparations or compositions in order to impart a hedonic effect. In other words, such a aldehyde compound, formate ester and/or alcohol, to be considered as being a perfuming one, must be recognized by a person skilled in the art of perfumery as being able to impart or modify in a positive or pleasant way the odor of a composition, and not just as having an odor. The perfuming aldehyde compound, formate ester and/or alcohol can be of natural or synthetic origin. Many of these perfuming aldehyde compounds, formate esters and/or alcohols are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

Herein described, the terms “perfuming aldehyde compound, formate ester and/or alcohol” are also referred to as “perfuming compounds”.

Practically, the invention is carried out exactly in the same manner, independently of the exact properties of the active aldehyde compound, formate ester or alcohol. Therefore, it is understood that, even if the invention will be further illustrated herein below with a specific reference to “perfuming compounds”, the below embodiments are also applicable to other active aldehyde compound, formate ester and/or alcohol (i.e. it is possible to replace the expression “perfuming” with “flavoring”, “malodor counteracting”, “antibacterial”, “antimicrobial”, “insect attractant” or with “insect repellent” for instance).

The term “optionally” is understood that a certain group to be optionally substituted can or cannot be substituted with a certain functional group. The term “one or more” is understood as being substituted with 1 to 7, preferably 1 to 5 and more preferably 1 to 3 of a certain functional group.

The terms “alkyl” and “alkenyl” are understood as comprising branched and linear alkyl and alkenyl groups. The terms “alkenyl” and “cycloalkenyl” are understood as comprising 1, 2 or 3 olefinic double bonds, preferably 1 or 2 olefinic double bonds, provided that the cycloalkenyl group is not an aryl group. The terms “cycloalkyl”, “cycloalkenyl”, “heterocycloalkyl” and “heterocyclic” are understood as comprising a monocyclic or fused, spiro and/or bridged bicyclic or tricyclic cycloalkyl, cycloalkenyl, heterocycloalkyl and heterocyclic groups, preferably monocyclic cycloalkyl, cycloalkenyl, and heterocycloalkyl groups. The term “alkoxy” is understood as —OR′ wherein R′ is a linear branched or cyclic alkyl group.

The term “aryl” is understood as comprising any group comprising at least one aromatic group such as phenyl, indenyl, indanyl, benzodioxolyl, dihydrobenzodioxinyl, tetrahydronaphthalenyl or naphthalenyl group.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynyl group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, optionally comprising . . . ” it is meant that said hydrocarbon group optionally comprises one two or three oxygen atoms in a form of alcohol, ketone, aldehyde, ether, ester, carboxylic acid, carbonate groups. These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain. For example, a —CH₂—CH₂—CHOH—CH₂— group represents a C₄ hydrocarbon group comprising an alcohol group (substitution of a hydrogen atom), i.e. a C₄ hydrocarbon comprising an oxygen atom; a —CH₂—CH₂—COO—CH₂—CH₂CH₂—CH₂— group represents a C₇ hydrocarbon group comprising one ester group (substitution of carbon atoms/insertion into the hydrocarbon chain), i.e. a C₇ hydrocarbon comprising two oxygen atoms and, similarly, a —CH₂—CH₂—O—CH₂—CH₂—O—CH₂—CH₂— group represents a C₆ hydrocarbon group comprising two ether groups, i.e. a C₆ hydrocarbon comprising two oxygen atoms.

The term “alpha to the formyloxy group” is understood as the carbon next to the formyloxy group. In other words, R² may not be a C(R^{2′})₂C(═O)OR^{2″} group wherein R^{2′} and R^{2″} are, independently from each other a hydrogen atom or a C₁₋₁₇ hydrocarbon group optionally comprising one oxygen atom. For compound of formula (IV), R² is a C₁₋₁₈ hydrocarbon group optionally comprising one, two or three oxygen atoms; provided that an ester functional group alpha to the hydroxy group is excluded By the term “allylic functional group” it is meant the normal meaning understood by a person skilled in the art, i.e. a —C—C═C— group optionally further substituted.

For the sake of clarity, by the expression “any one of its stereoisomers or a mixture thereof”, or the similar, it is meant the normal meaning understood by a person skilled in the art, i.e. that the compound of formula (I) can be a pure enantiomer or diastereomer. In other words, the compound of formula (I) may possess several stereocenters and each of said stereocenter can have two different stereochemistries (e.g. R or S). The compound of formula (I) may even be in the form of a pure enantiomer or in the form of a mixture of enantiomers or diastereoisomers. The compound of formula (I) can be in a racemic form or scalemic form. Therefore, the compound of formula (I) can be one stereoisomer or in the form of a composition of matter comprising, or consisting of, various stereoisomers.

According to any one of the above embodiments of the invention, said compound of formula (I) can be in the form of its E or Z isomer or of a mixture thereof, e.g. the invention comprises compositions of matter consisting of one or more compounds of formula (I), having the same chemical structure but differing by the configuration of the double bond. In particular, compound (I) can be in the form of a mixture consisting of isomers E and Z and wherein said isomers E represent at least 50% of the total mixture, or even at least 60%, or even at least 70%, or even at least 75% (i.e a mixture E/Z comprised between 75/25 and 100/0). Or, compound (I) can be in the form of a mixture consisting of isomers E and Z and wherein said isomers Z represent at least 50% of the total mixture, or even at least 60%, or even at least 70%, or even at least 75% (i.e a mixture E/Z comprised between 25/75 and 0/100).

According to any one of the embodiments of the invention, the heterocycloalkyl group is a vyvloalkyl comprising at least one heteroatom wherein the heteroatom represents one or more of an oxygen atom.

According to any one of the embodiments of the invention, when R¹ may be a C₃₋₁₅ alkenyl, it is understood that the double bond is not adjacent to the carbon connecting R¹. In other words, compounds of formula (II) are not an enal and compounds of formula (I) are not a dienol ether.

According to any embodiments of the invention, the compound of formula (I) is a C₁₈-C₃₆ compound, preferably a C₂₀₋₃₆ compound.

According to a particular embodiment of the invention, R¹ may be a C₁₋₁₅ alkyl, C₃₋₁₅ alkenyl, C₃₋₁₅ cycloalkyl, C₅₋₁₅ cycloalkenyl or C₃₋₁₄ heterocycloalkyl group, each optionally substituted with one or more of a hydroxy, C₁₋₁₅ alkyl, C₂₋₁₅ alkenyl, C₁₋₁₅ alkoxy, C₂₋₁₅ alkenyloxy, C₃₋₁₅ cycloalkyl, C₅₋₁₅ cycloalkenyl, C₃₋₁₅ heterocycloalkyl, C₆₋₁₀ aryl and/or C₆₋₁₀ aryloxy group, each optionally substituted with one or more of a C₁₋₈ alkyl, C₁₋₈ alkoxy and or hydroxy group. Particularly, R¹ may represent a C₁₋₁₂ alkyl, C₃₋₁₂ alkenyl, C₃₋₁₂ cycloalkyl, C₅₋₁₂ cycloalkenyl or C₃₋₁₂ heterocycloalkyl group, each optionally substituted with one or more of a hydroxy, C₁₋₁₀ alkyl, C₂₋₁₀ alkenyl, C₁₋₁₀ alkoxy, C₂₋₁₀ alkenyloxy, C₃₋₁₀ cycloalkyl, C₅₋₁₀ cycloalkenyl, C₃₋₁₀ heterocycloalkyl C₆₋₁₀ aryl and/or C₆₋₁₀ aryloxy group, each optionally substituted with one or more of a C₁₋₆ alkyl, C₁₋₆ alkoxy and/or hydroxy group. Particularly, R¹ may represent a C₁₋₁₀ alkyl, C₃₋₁₀ alkenyl, C₃₋₁₁ cycloalkyl or C₅₋₁₁ cycloalkenyl group, each optionally substituted with one or more of a hydroxy, C₁₋₄ alkyl, C₁₋₄ alkoxy, C₃₋₈ cycloalkyl, C₅₋₈ cycloalkenyl, C₆ aryl and/or C₆ aryloxy group, each optionally substituted with one or more of a hydroxy, C₁₋₄ alkyl and/or C₁₋₄ alkoxy group. Particularly, R¹ may represent a C₁₋₁₀ alkyl, C₃₋₁₀ alkenyl group, each optionally substituted with one or more C₁₋₄ alkoxy, 4-methoxyphenyl and/or phenyl group. Particularly, R¹ may represent a nonyl, decyl, undecyl, 2-undecyl, benzyl, dec-8-en-1-yl dec-9-en-1-yl, 4-phenylbutan-2-yl, 4-(C₁₋₆ alkyl)benzyl wherein the alkyl group is optionally substituted by a hydroxy or methoxy group, 4-phenylbutan-2-yl or phenylethyl group. Even more particularly, R¹ may represent a heptyl, nonyl, decyl, undecyl, 2-undecyl, benzyl, non-3-en-1-yl, non-8-en-1-yl, dec-8-en-1-yl, dec-9-en-1-yl, 4-phenylbutan-2-yl, 4-(tert-butyl)benzyl, 2-phenylprop-1-yl, 2-(4-methylcyclohex-3-en-1-yl)propyl, 2,6-dimethyhept-5-en-1-yl, 1-(4-(tert-butyl)phenyl)propan-2-yl, 2,4-dimethylcyclohex-3-en-1-yl, 2-(4,4-dimethylcyclohex-1-en-1-yl)ethyl, 4-phenylbutan-2-yl or phenylethyl group.

According to a particular embodiment of the invention, R¹ may comprise at least, 5, 6, or even 7 carbon atoms.

According to any one of the embodiments of the invention, R¹ is not a 2-hexylidenecyclopentyl group.

Herein disclosed, R² may represent a C₃₋₁₈ hydrocarbon group optionally comprising one or two oxygen atoms. Particularly, R² may represent a C₄₋₁₈ hydrocarbon group optionally comprising one or two oxygen atoms.

According to any one of the embodiments of the invention, R² may represent a C₅₋₁₈ hydrocarbon group optionally comprising one or two oxygen atoms. Particularly, R² may represent a C₆₋₁₈ hydrocarbon group optionally comprising one or two oxygen atoms. Particularly, R² may represent a C₆₋₁₈ hydrocarbon group optionally comprising one or two oxygen atoms; provided that R² does not comprise an allylic functional group. Particularly, R² may represent a C₆₋₈ hydrocarbon group optionally comprising one or two oxygen atoms; provided that R² is not a benzyl group and does not comprise an allylic functional group. Even more particularly, R² may represent a C₆₋₈ hydrocarbon group optionally comprising one or two oxygen atoms; provided that R² is not a benzyl group or a cyclohexyl group or 2-hydroxy-1,2-diphenylethyl, or a 1-(tert-butoxy)-7,7-dimethylbicyclo[2.2.1]heptan-2-yl group and does not comprise an allylic functional group.

According to any one of the embodiments of the invention, R² is not a benzyl group or a cyclohexyl group or 2-hydroxy-1,2-diphenylethyl, or a 1-(tert-butoxy)-7,7-dimethylbicyclo[2.2.1]heptan-2-yl group and does not comprise an allylic functional group.

According to any one of the embodiments of the invention, R² comprises at least 6 carbon atoms.

According to any one of the embodiments of the invention, R² may represent a C₁₋₁₈ alkyl, C₂₋₁₈ alkenyl, C₃₋₁₈ cycloalkyl or C₅₋₁₈ cycloalkenyl group, each optionally substituted with one or more of a hydroxy, C₁₋₁₅ alkyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, hydroxy and/or carboxylic acid. Particular, R² may represent a C₁₋₁₅ alkyl, C₂₋₁₅ alkenyl, C₃₋₁₅ cycloalkyl or C₅₋₁₅ cycloalkenyl group, each optionally substituted with one or more of a hydroxy, C₁₋₁₅ alkyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, hydroxy and/or carboxylic acid. Particularly, R² may represent a C₁₋₁₂ alkyl, C₃₋₁₂ alkenyl, C₃₋₁₂ cycloalkyl or C₅₋₁₂ cycloalkenyl group, each optionally substituted with one or more of a hydroxy, C₁₋₁₀ alkyl, C₁₋₁₀ alkoxy, C₃₋₁₀ cycloalkyl, C₅₋₁₀ cycloalkenyl, C₆₋₁₀ aryl and/or C₆₋₁₀ aryloxy group, each optionally substituted with one or more of a C₁₋₆ alkyl, C₁₋₆ alkoxy and/or hydroxy group. Particularly, R² may represent a C₁₋₁₀ alkyl, C₃₋₁₀ alkenyl, C₃₋₁₁ cycloalkyl or C₅₋₁₁ cycloalkenyl group, each optionally substituted with one or more of a hydroxy, C₁₋₄ alkyl, C₁₋₄ alkoxy, C₃₋₈ cycloalkyl, C₅₋₈ cycloalkenyl, C₆ aryl and/or C₆ aryloxy group, each optionally substituted with one or more of a hydroxy, C₁₋₄ alkyl and/or C₁₋₄ alkoxy group. Particularly, R² may represent a C₈₋₁₀ alkyl group, a C₆₋₁₀ alkenyl group having one olefinic double bond or a C₂ alkyl substituted with one phenyl or C₆ aryloxy group. Particularly, R² may represent an octyl, 2-phenoxyethyl, 3,7-dimethyloctyl, octan-2-yl, octan-3-yl, 3,7-dimethyloct-6-en-1-yl, (Z)-hex-3-en-1-yl, (Z)-oct-3-en-1-yl or 2-phenylethyl group. Even more particularly, R² may represent a 2-phenoxyethyl or 2-phenylethyl group.

According to any one of the embodiments of the invention, R² may represent C₆₋₁₀ aryl, optionally substituted with one or more of a hydroxy, C₁₋₆ alkyl, C₂₋₆ alkenyl, C₁₋₆ alkoxy, C₂₋₆ alkenyloxy and/or —COOR³ group wherein R³ is a C₁₋₆ alkyl, C₂₋₆ alkenyl or a benzyl group. Particularly, R² may represent C₆ aryl, optionally substituted with one or more of a hydroxy, C₁₋₄ alkyl, C₂₋₄ alkenyl, C₁₋₄ alkoxy, C₂₋₄ alkenyloxy and/or —COOR³ group wherein R³ is a C₁₋₆ alkyl, C₂₋₆ alkenyl or a benzyl group. Even more particularly, R² may represent C₆ aryl, optionally substituted with one or more of a hydroxy, C₁₋₃ alkyl, C₂₋₃ alkenyl, C₁₋₃ alkoxy, C₂₋₃ alkenyloxy and/or —COOR³ group wherein R³ is a C₁₋₆ alkyl, C₂₋₆ alkenyl or a benzyl group.

According to a particular embodiment, at least one of the compounds of formula (II), (III) and (IV) are active compounds. Even more, the compound of formula (II) is active compound.

According to a particular embodiment, the aldehyde compound of formula (II) and/or the active alcohol of formula (IV) are perfuming ingredients. For a person skilled in the art it is also evident that compounds of formula (II), (III) and (IV) according to the present invention are inherently volatile compounds.

The aldehyde compound, formate ester and/or alcohol may be advantageously characterized by a vapor pressure above 1.0 Pa, as obtained by calculation using the software EPIwin v. 3.10 (2000, available at the US Environmental Protection Agency). According to another embodiment, the vapor pressure of the ketone, formate ester and/or alcohol may be above 5.0, or even above 7.0 Pa.

According to a particular embodiment, the compound of formula (I) is non-volatile. The compound of formula (I) may be advantageously characterized by a vapor pressure below 0.01 Pa, as obtained by calculation using the software EPIwin v. 3.10 (2000, available at the US Environmental Protection Agency). According to a preferred embodiment, the vapor pressure is below 0.001 Pa.

According to a particular embodiment, the aldehyde compound of formula (II) is selected from the group consisting of hexanal, heptanal, octanal, nonanal, decanal, undecanal, dodecanal, 2-ethylhexanal, 3,7-dimethyloctanal, 2-methyldecanal, 2-methylundecanal, 6-nonenal, 4-decenal, 5-octenal, 8-nonenal, 8-decenal, 9-decenal, 3-(3,3-dimethyl-2,3-dihydro-1H-inden-5-yl)propanal, 9-undecenal, 10-undecenal, 3-(6,6-dimethylbicyclo[3.1.1]hept-2-en-2-yl)propanal, 4-dodecenal, 8-isopropyl-6-methylbicyclo[2.2.2]oct-5-ene-2-carbaldehyde, 3-(4-(tert-butyl)phenyl)propanal, 3-(4-(tert-butyl)phenyl)-2-methylpropanal, 2-methyl-4-phenylbutanal, 3-methyl-5-phenylpentanal, 4-(4-hydroxy-4-methylpentyl)cyclohex-3-ene-1-carbaldehyde, 2,4-dimethylcyclohex-3-ene-1-carbaldehyde, 3-phenylbutanal, 2,6-dimethylhept-5-enal, 3-(4-methylcyclohex-3-en-1-yl)butanal, 3-(4,4-dimethylcyclohex-1-en-1-yl)propanal, 4-methyl-5-(p-tolyl)pent-4-enal, 3,7-dimethyloct-6-enal, 2-phenylpropanal, phenylacetaldehyde, 3-(benzo[d][1,3]dioxol-5-yl)-2-methylpropanal, 5-methoxyoctahydro-1H-4,7-methanoindene-1-carbaldehyde, 6-methoxyoctahydro-1H-4,7-methanoindene-1-carbaldehyde, 3-(3-isopropylphenyl)butanal, 3-(4-isobutyl-2-methylphenyl)propanal, 3,6-dimethylcyclohex-3-ene-1-carbaldehyde, 2-((3,7-dimethyloct-6-en-1-yl)oxy)acetaldehyde, 3-(4-ethylphenyl)-2,2-dimethylpropanal, 3,5,6-trimethylcyclohex-3-ene-1-carbaldehyde, 4-(4-methylpent-3-en-1-yl)cyclohex-3-ene-1-carbaldehyde, 3-phenylpropanal, 3-(4-isopropylphenyl)-2-methylpropanal, 8,8-dimethyl-1,2,3,4,6,7,8,8a-octahydronaphthalene-2-carbaldehyde, 2-methyl-4-(2,2,3-trimethylcyclopent-3-en-1-yl)pent-4-enal), 3,5,5-trimethylhexanal, 2,6,10-trimethyl-9-undecenal, 3-(4-methoxyphenyl)-2-methylpropanal, 7-hydroxy-3,7-dimethyloctanal, 3-(4-isopropylphenyl)propanal, 2-(4-isopropylphenyl)acetaldehyde, 2-(4-(tert-butyl)phenyl)acetaldehyde, 6-methoxy-2,6-dimethylheptanal, 2,6-dimethyl-5-heptenal, 3-(4-isopropylcyclohex-1-en-1-yl)propanal, 3-(4-isopropylcyclohexylidene)propanal, 2,4-dimethyl-3-cyclohexen-1-carbaldehyde, 3-(3-isopropylcyclohex-1-en-1-yl)propanal, 3-(5-isopropylcyclohex-1-en-1-yl)propanal, 4-(4-methyl-3-pentenyl)-3-cyclohexene-1-carbaldehyde, 5-cyclohexyl-2,4-dimethyl-4-pentenal, 5,9-dimethyl-4-decenal, and 1-methyl-4-(4-methyl-3-pentenyl)-3-cyclohexen-1-carbaldehyde.

Particularly, the aldehyde compound of formula (II) is selected from the group consisting of octanal, nonanal, decanal, undecanal, dodecanal, 2-methyldecanal, 2-methylundecanal, 2-phenylpropanal, 2-methyl-4-phenylbutanal, phenylacetaldehyde, 2-(4-(tert-butyl)phenyl)acetaldehyde, phenylacetaldehyde, 4-decenal, 4-dodecenal, 9-decenal, 9-undecenal, 10-undecenal, 2,6-dimethyl-5-heptenal, 2,4-dimethyl-3-cyclohexen-1-carbaldehyde, 3-phenylbutanal, 3-(4,4-dimethylcyclohex-1-en-1-yl)propanal, 4-(4-methylpent-3-en-1-yl)cyclohex-3-ene-1-carbaldehyde, 3-(3,3-dimethyl-2,3-dihydro-1H-inden-5-yl)propanal, 3-(4-methylcyclohex-3-en-1-yl)butanal, 3-(6,6-dimethylbicyclo[3.1.1]hept-2-en-3-yl)propanal, 8-isopropyl-6-methylbicyclo[2.2.2]oct-5-ene-2-carbaldehyde, 3-(4-(tert-butyl)phenyl)-2-methylpropanal, and 3,7-dimethyloct-6-enal.

According to a particular embodiment, the formate ester of formula (III) are selected from the group consisting of butyl formate, pentyl formate, 2-methylbutyl formate, 3-methylbutyl formate, butan-2-yl formate, 2-methylpropyl formate, cyclohexyl formate, hexyl formate, heptyl formate, octyl formate, nonyl formate, decyl formate, undecyl formate, dodecyl formate, trideceyl formate, tetradecyl formate, 2-hexyl formate, 3-hexyl formate, 3-octyl formate, 2-octyl formate, 3-octen-1-yl formate, benzyl formate, 9-decen-1-yl formate, 3,7-dimethyloctyl formate, 3,7-dimethyloct-6-enyl formate, 3,7-dimethyloct-7-enyl formate, 4-methoxybenzyl formate, 3-hexenyl formate, 3,5,5-trimethylhexyl formate, 2-phenylethyl formate, 2-(phenoxy)ethyl formate, 3-phenylpropyl formate, 2-phenylpropan-1-yl formate, 1-phenylethyl formate, 4-phenylbutanyl formate, (Z)-6-nonen-1-yl formate, bornyl formate, isobornyl formate, cedryl formate, cyclododecyl formate, decahydronaphthalen-2-yl formate, menthyl formate, 5-methyl-2-(prop-1-en-2-yl)cyclohexyl formate, 3-methyl-5-phenylpentanyl formate, (4-isopropylcyclohexyl)methanyl formate, 2-pentyl-1-cyclopentyl formate, 5-ethyl-2-nonyl formate, (4-tert-butyl)cyclohexyl formate, 2-methoxy-4-propylcyclohexyl formate, 3a,4,5,6,7,7a-hexahydro-1H-4,7-methanoinden-5-yl formate, 3a,4,5,6,7,7a-hexahydro-1H-4,7-methanoinden-6-yl formate, 1-(3,3-dimethylcyclohexyl)ethyl formate, 2-methyl-1-phenylpropan-2-yl formate, 2,6-dimethyloct-7-en-2-yl formate, 2,6-dimethyloctan-2-yl formate, 3,7-dimethyloctanyl formate, 4-cyclohexyl-2-methyl-2-butanyl formate, (2,5-dimethyl-2,3-dihydro-1H-inden-2-yl)methanyl formate, 1-((2-(tert-butyl)cyclohexyl)oxy)butan-2-yl formate, 1-((1RS,6SR)-2,2,6-trimethylcyclohexyl)hexan-3-yl formate, 2,6-dimethyl-2-heptanol, 2-methyl-4-[(1R)-2,2,3-trimethyl-3-cyclopenten-1-yl]-4-penten-1-yl formate, 2-methyl-1-phenylpropan-2-yl foramte, (1RS,2SR,5RS)-2-isopropyl-5-methylcyclohexyl formate and 4-methyl-6-phenyl-2-hexanyl formate.

In a more particular embodiment, the formate ester of formula (III) is selected from the group consisting of 2-phenylethyl formate, 3-hexenyl formate, octyl formate, decyl formate, 3,7-dimethyloct-6-en-1-yl formate, 3,7-dimethyloct-7-enyl formate, 2-phenoxyethyl formate, hexyl formate, benzyl formate, octan-3-yl formate, octan-2-yl formate, (1RS,2SR,5RS)-2-isopropyl-5-methylcyclohexyl formate, cyclododecyl formate, 1-(3,3-dimethylcyclohexyl)ethyl formate, 1-((2-(tert-butyl)cyclohexyl)oxy)butan-2-yl formate, 2,6-dimethyloct-7-en-2-yl formate, 3,7-dimethyloctan-3-yl formate, 2-methyl-1-phenylpropan-2-yl formate, and 2,6-dimethylheptan-2-yl formate.

In a particular embodiment, the alcohol of formula (IV) is selected from the group consisting of butanol, pentanol, 2-methylbutanol, 3-methylbutanol, butan-2-ol, 2-methylpropanol, cyclohexanol, hexanol, heptanol, octanol, nonanol, decanol, 1-undecanol, 1-dodecanol, 1-tridecanol, 1-tetradecanol, 2-hexanol, 3-hexanol, 3-octanol, 2-octanol, 3-octenol, benzyl alcohol, 9-decen-1-ol, 3,7-dimethyloctan-1-ol, 3,7-dimethyloct-6-en-1-ol, 3,7-dimethyloct-7-en-1-ol, 4-methoxybenzyl alcohol, 3-hexen-1-ol, 3,5,5-trimethylhexanol, 2-phenylethanol, 2-(phenoxy)ethanol, 3-phenylpropanol, 2-phenylpropan-1-ol, 1-phenylethan-1-ol, 4-phenylbutan-2-ol, (Z)-6-nonen-1-ol, borneol, isoborneol, cedrol, cyclododecanol, decahydronaphthalen-2-ol, menthol, 1-phenylethanol, 5-methyl-2-(prop-1-en-2-yl)cyclohex-1-ol, 3-methyl-5-phenylpentan-1-ol, (4-isopropylcyclohexyl)methanol, 2-pentyl-1-cyclopentanol, 5-ethyl-2-nonanol, 4-(tert-butyl)cyclohexan-1-ol, 2-methoxy-4-propylcyclohexan-1-ol, 3a,4,5,6,7,7a-hexahydro-1H-4,7-methanoinden-5-ol, 3a,4,5,6,7,7a-hexahydro-1H-4,7-methanoinden-6-ol, 1-(3,3-dimethylcyclohexyl)ethanol, 2-methyl-1-phenylpropan-2-ol, 3,7-dimethylocta-1,6-dien-3-ol, 2,6-dimethyloct-7-en-2-ol, 2,6-dimethyloctan-2-ol, 3,7-dimethyloctan-3-ol, 4-cyclohexyl-2-methyl-2-butanol, (2,5-dimethyl-2,3-dihydro-1H-inden-2-yl)methanol, 1-((2-(tert-butyl)cyclohexyl)oxy)butan-2-ol, 1-((1RS,6SR)-2,2,6-trimethylcyclohexyl)hexan-3-ol, 2,6-dimethyl-2-heptanol, 2-methyl-4-[(1R)-2,2,3-trimethyl-3-cyclopenten-1-yl]-4-penten-1-ol, 2-methyl-1-phenylpropan-2-ol, (1RS,2SR,5RS)-2-isopropyl-5-methylcyclohexanol and 4-methyl-6-phenyl-2-hexanol

In a more preferred embodiment, the alcohol of formula (IV) is selected from the group consisting of 1-hexanol, 1-heptanol, 1-octanol, 1-nonanol, 2-octanol, 3-octanol, 1-decanol, 1-undecanol, 1-dodecanol, benzyl alcohol, 3,7-dimethyloct-6-en-1-ol, 3,7-dimethyloct-7-en-1-ol, 3,7-dimethyloctan-1-ol, 3-hexen-1-ol, 3-octen-1-ol, 2-phenylethanol, 2-(phenoxy)ethanol, 9-decen-1-ol, 2,6-dimethyloct-7-en-2-ol, (2,5-dimethyl-2,3-dihydro-1H-inden-2-yl)methanol, and cyclododecanol.

According to a particular embodiment, the compound of formula (I) is selected from the group consisting of (2-(non-1-en-1-yloxy)ethyl)benzene, (2-(undec-1-en-1-yloxy)ethyl)benzene, (2-(dodec-1-en-1-yloxy)ethyl)benzene, (2-(tridec-1-en-1-yloxy)ethyl)benzene, (2-(undeca-1,10-dien-1-yloxy)ethyl)benzene, ((3-phenethoxyallyl)benzene), (4-phenethoxybut-3-en-2-yl)benzene, (3-methyl-5-phenethoxypent-4-en-1-yl)benzene, 1-(octyloxy)dodec-1-ene, 1-(decyloxy)dodec-1-ene, 1-(hex-3-en-1-yloxy)dodec-1-ene, 1-((3,7-dimethyloct-6-en-1-yl)oxy)dodec-1-ene, 1-((3,7-dimethyloctyl)oxy)undec-1-ene, 1-((3,7-dimethyloct-6-en-1-yl)oxy)non-1-ene, (2-(undec-1-en-1-yloxy)ethoxy)benzene, (2-(dodec-1-en-1-yloxy)ethoxy)benzene, 1-(octan-2-yloxy)dodec-1-ene, 1-(tert-butyl)-4-(3-phenethoxyallyl)benzene, (2-((3-methylundec-1-en-1-yl)oxy)ethyl)benzene, (2-((3-methyldodec-1-en-1-yl)oxy)ethoxy)benzene, (2-((3-methyldodec-1-en-1-yl)oxy)ethyl)benzene, 1-(hex-3-en-1-yloxy)-3-methyldodec-1-ene, 3-methyl-1-(oct-3-en-1-yloxy)dodec-1-ene, 3-methyl-1-(octyloxy)dodec-1-ene, 3-methyl-1-(octan-3-yloxy)dodec-1-ene, 1-((3,7-dimethyloct-6-en-1-yl)oxy)-3-methyldodec-1-ene, 1-((3,7-dimethyloctyl)oxy)-3-methyldodec-1-ene, (((3-methyldodec-1-en-1-yl)oxy)methyl)benzene, (2-(dodeca-1,10-dien-1-yloxy)ethyl)benzene, 1-(hex-3-en-1-yloxy)dodeca-1,10-diene, 1-(octyloxy)dodeca-1,10-diene, 1-(decyloxy)dodeca-1,10-diene, 1-((3,7-dimethyloct-6-en-1-yl)oxy)dodeca-1,10-diene, (2-(dodeca-1,11-dien-1-yloxy)ethyl)benzene, 1-(octyloxy)dodeca-1,11-diene, 1-(decyloxy)dodeca-1,11-diene, 1-(octyloxy)tridec-1-ene, 1-(decyloxy)tridec-1-ene, (5-phenethoxypent-4-en-2-yl)benzene, (2-((2-(2,4-dimethylcyclohex-3-en-1-yl)vinyl)oxy)ethyl)benzene, 1,1-dimethyl-6-(4-phenethoxybut-3-en-1-yl)-2,3-dihydro-1H-indene, 5-isopropyl-2-methyl-7-(2-phenethoxyvinyl)bicyclo[2.2.2]oct-2-ene, (2-(trideca-1,5-dien-1-yloxy)ethyl)benzene, (2-(undeca-1,5-dien-1-yloxy)ethyl)benzene, 1-(tert-butyl)-4-(4-phenethoxybut-3-en-1-yl)benzene, 1-(tert-butyl)-4-(2-methyl-4-phenethoxybut-3-en-1-yl)benzene, (2-((3,7-dimethylocta-1,6-dien-1-yl)oxy)ethyl)benzene, (2-((4,8-dimethylnona-1,7-dien-1-yl)oxy)ethyl)benzene, (2-((4-(4,4-dimethylcyclohex-1-en-1-yl)but-1-en-1-yl)oxy)ethyl)benzene and (2-((4-(4-methylcyclohex-3-en-1-yl)pent-1-en-1-yl)oxy)ethyl)benzene.

According to any one of the above embodiments, the aldehyde compound of formula (II), the formate ester of formula (III) and the alcohol of formula (IV) are released from the precursor compound of formula (I) via oxidation of the precursor compound of formula (I) under ambient conditions. Even more, the precursor compound of formula (I) is oxidized under ambient conditions and in absence of any catalyst. For the sake of clarity, by the expression “ambient conditions”, or the similar, it is meant the normal meaning understood by a person skilled in the art, i.e. the oxidation occurs at room temperature, under air and atmospheric pressure. In other words, the environment wherein the compound is oxidized is air. Herewith it is understood, that the compound of formula (I) is oxidized in ambient air. In particular, it is understood that the compound of formula (I) does not require a pure oxygen environment, heat or catalyst to be oxidized.

Without intending to be limited to any particular theory, the rate at which the precursor compound of formula (I) is oxidized may be greater than, equal to, or slower than the evaporation rates of the individual aldehyde compound of formula (II), the formate esters of formula (III) or the alcohols of formula (IV).

In some embodiments, the rate at which the precursor compound of formula (I) is oxidized, and thereby, the rate at which the individual aldehyde compound of formula (II), the formate esters of formula (III) or the alcohols of formula (IV) are released intensifies or prolongs the diffusion effect, and/or perception of the characteristic fragrance of at least one aldehyde compound formula (II), of at least one formate ester of formula (III) and/or of at least one alcohol of formula (IV) as defined above.

In one embodiment, 100% of the compound of formula (I) is oxidized in ambient air in a period of time ranging from 24 to 48 hours. Alternatively, 90% of the compound of formula (I) is oxidized in ambient air in a period of time ranging from 24 to 48 hours. Alternatively, 80% of the compound of formula (I) is oxidized in ambient air in a period of time ranging from 24 to 48 hours. Alternatively, 70% of the compound of formula (I) is oxidized in ambient air in a period of time ranging from 24 to 48 hours. Alternatively, 60% of the compound of formula (I) is oxidized in ambient air in a period of time ranging from 24 to 48 hours. Alternatively, 50% of the compound of formula (I) is oxidized in ambient air in a period of time ranging from 24 to 48 hours. Alternatively, 40% of the compound of formula (I) is oxidized in ambient air in a period of time ranging from 24 to 48 hours. Alternatively, 30% of the compound of formula (I) is oxidized in ambient air in a period of time ranging from 24 to 48 hours. Alternatively, 20% of the compound of formula (I) is oxidized in ambient air in a period of time ranging from 24 to 48 hours. Alternatively, 10% of the compound of formula (I) is oxidized in ambient air in a period of time ranging from 24 to 48 hours. Alternatively, 9% of the compound of formula (I) is oxidized in ambient air in a period of time ranging from 24 to 48 hours. Alternatively, 8% of the compound of formula (I) is oxidized in ambient air in a period of time ranging from 24 to 48 hours. Alternatively, 7% of the compound of formula (I) is oxidized in ambient air in a period of time ranging from 24 to 48 hours. Alternatively, 6% of the compound of formula (I) is oxidized in ambient air in a period of time ranging from 24 to 48 hours. Alternatively, 5% of the compound of formula (I) is oxidized in ambient air in a period of time ranging from 24 to 48 hours. Alternatively, 4% of the compound of formula (I) is oxidized in ambient air in a period of time ranging from 24 to 48 hours. Alternatively, 3% of the compound of formula (I) is oxidized in ambient air in a period of time ranging from 24 to 48 hours. Alternatively, 2% of the compound of formula (I) is oxidized in ambient air in a period of time ranging from 24 to 48 hours. Alternatively, 1% of the compound of formula (I) is oxidized in ambient air in a period of time ranging from 24 to 48 hours.

The present invention also relates to a microcapsule comprising at least one compound of formula (I). In one embodiment, the at least one compound of formula (I) is encapsulated in a core-shell microcapsule wherein the at least one compound of formula (I) is contained in the core surrounded by the shell. In one embodiment, the shell of the microcapsule protects the compound of formula (I) from the environment. The shell is made of material which is able to release the at least one compound of formula (I) and/or the compound of formulas (II), (III) and/or (IV). In one embodiment, the shell is made of material which is able to release the compound of formula (I) and/or the compound of formulas (II), (III) and/or (IV) upon breakage of the shell and/or by diffusion through the shell. A person skilled in the art is well aware of processes to prepare said microcapsules. So, microcapsule comprising at least one compound of formula (I) is one object of the present invention.

In a preferred embodiment, encapsulation of a compound of formula (I) may provide an environment within the capsule wherein all, or a portion of the compound of formula (I) may oxidize, thereby releasing the individual aldehyde of formula (II), the formate esters of formula (III) or the alcohols of formula (IV) into the capsule. In a preferred embodiment, the shell of the microcapsule may act as a permeability barrier, preventing the leakage of the individual aldehyde compound of formula (II), the formate esters of formula (III) or the alcohols of formula (IV) from the capsule.

According to a particular embodiment, the shell of the microcapsule comprises a material selected from the group consisting of polyurea, polyurethane, polyamide, polyester, poly(meth)acrylate (i.e. polyacrylate and/or polymethacrylate), polysiloxane, polycarbonate, polysulfonamide, polymers of urea and formaldehyde, melamine and formaldehyde, melamine and urea, or melamine and glyoxal and mixtures thereof. The shell can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to a particular embodiment, the core-shell microcapsule(s) can be also derived by using different or more than one encapsulation method.

In a preferred embodiment, the shell of the microcapsules may be, each independently, selected from the group of aminoplast, polyamide, polyester, polyurea and polyurethane shells and mixtures thereof.

In a particular embodiment, the shell of the microcapsules comprises an aminoplast copolymer, such as melamine-formaldehyde or urea-formaldehyde or cross-linked melamine formaldehyde or melamine glyoxal.

In a particular embodiment, the shell of the microcapsules is polyurea-based made from, for example but not limited to isocyanate-based monomers and amine-containing crosslinkers such as guanidine carbonate and/or guanazole. Certain polyurea microcapsules comprise a polyurea wall which is the reaction product of the polymerisation between at least one polyisocyanate comprising at least two isocyanate functional groups and at least one reactant selected from the group consisting of an amine (for example a water-soluble guanidine salt and guanidine); a colloidal stabilizer or emulsifier; and an encapsulated perfume. However, the use of an amine can be omitted.

In a particular embodiment, the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). In a particular embodiment, the emulsifier is an anionic or amphiphilic biopolymer, which may be for example chosen from the group consisting of gum Arabic, soy protein, gelatin, sodium caseinate and mixtures thereof.

In a particular embodiment, the shell of the microcapsules is polyurethane-based made from, for example but not limited to polyisocyanate and polyols, polyamide, polyester, etc.

In a particular embodiment, the microcapsules have a polymeric shell resulting from complex coacervation wherein the shell is possibly cross-linked.

In a particular embodiment of the core-shell microcapsules, the core-shell microcapsules comprise an oil-based core comprising a hydrophobic active, preferably at least one compound of formula(I), and a composite shell comprising a first material and a second material, wherein the first material and the second material are different, the first material is a coacervate, the second material is a polymeric material.

In a particular embodiment, the weight ratio between the first material and the second material is comprised between 50:50 and 99.9:0.1.

In a particular embodiment, the coacervate comprises a first polyelectrolyte, preferably selected among proteins (such as gelatin), polypeptides or polysaccharides (such as chitosan), most preferably Gelatin and a second polyelectrolyte, preferably alginate salts, cellulose derivatives guar gum, pectinate salts, carrageenan, polyacrylic and methacrylic acid or xanthan gum, or yet plant gums such as acacia gum (Gum Arabic), most preferably Gum Arabic.

The coacervate first material can be hardened chemically using a suitable cross-linker such as glutaraldehyde, glyoxal, formaldehyde, tannic acid or genipin or can be hardenedenzymatically using an enzyme such as transglutaminase.

The second polymeric material can be selected from the group consisting of polyurea, polyurethane, polyamide, polyester, polyacrylate, polysiloxane, polycarbonate, polysulfonamide, polymers of urea and formaldehyde, melamine and formaldehyde, melamine and urea, or melamine and glyoxal and mixtures thereof, preferably polyurea and/or polyurethane. The second material is preferably present in an amount less than 3 wt. %, preferably less than 1 wt. % based on the total weight of the microcapsule slurry.

The preparation of an aqueous dispersion/slurry of core-shell microcapsules is well known by a skilled person in the art. In a particular embodiment, the microcapsule wall material may comprise any suitable resin and especially including melamine, glyoxal, polyurea, polyurethane, polyamide, polyester, etc. Suitable resins include the reaction product of an aldehyde and an amine, suitable aldehydes include, formaldehyde and glyoxal. Suitable amines include melamine, urea, benzoguanamine, glycoluril, and mixtures thereof. Suitable melamines include, methylol melamine, methylated methylol melamine, imino melamine and mixtures thereof. Suitable ureas include, dimethylol urea, methylated dimethylol urea, urea-resorcinol, and mixtures thereof. Suitable materials for making may be obtained from one or more of the following companies Solutia Inc. (St Louis, Missouri U.S.A.), Cytec Industries (West Paterson, New Jersey U.S.A.), Sigma-Aldrich (St. Louis, Missouri U.S.A.).

In a particular embodiment of the core-shell microcapsules, the core-shell microcapsules comprises
an oil-based core comprising a hydrophobic active, preferably comprising at least one compound of formula (I),
optionally an inner shell made of a polymerized polyfunctional monomer;
a biopolymer shell comprising a protein, wherein at least one protein is cross-linked.

According to a particular embodiment, the protein is chosen in the group consisting of milk proteins, caseinate salts such as sodium caseinate or calcium caseinate, casein, whey protein, hydrolyzed proteins, gelatins, gluten, pea protein, soy protein, silk protein and mixtures thereof, preferably sodium caseinate, most preferably sodium caseinate

According to a particular embodiment, the protein comprises sodium caseinate and a globular protein, preferably chosen in the group consisting of whey protein, beta-lactoglobulin, ovalbumine, bovine serum albumin, vegetable proteins, and mixtures thereof.

The protein is preferably a mixture of sodium caseinate and whey protein.

According to a particular embodiment, the biopolymer shell comprises a crosslinked protein chosen in the group consisting of sodium caseinate and/or whey protein.

According to a particular embodiment, the microcapsules slurry comprises at least one microcapsule made of:
an oil-based core comprising the hydrophobic active, preferably comprising at least one compound of formula (I);
an inner shell made of a polymerized polyfunctional monomer; preferably a polyisocyanate having at least two isocyanate functional groups
a biopolymer shell comprising a protein, wherein at least one protein is cross-linked; wherein the protein contains preferably a mixture comprising sodium caseinate and a globular protein, preferably whey protein.
optionally at least an outer mineral layer.

According to an embodiment, sodium caseinate and/or whey protein is (are) cross-linked protein(s).

The weight ratio between sodium caseinate and whey protein is preferably comprised between 0.01 and 100, preferably between 0.1 and 10, more preferably between 0.2 and 5.

In a particular embodiment, the microcapsules is a one-shell aminoplast core-shell microcapsule obtainable by a process comprising the steps of:
1) admixing a perfume oil with at least a polyisocyanate having at least two isocyanate functional groups to form an oil phase;
2) dispersing or dissolving into water an aminoplast resin and optionally a stabilizer to form a water phase;
3) preparing an oil-in-water dispersion, wherein the mean droplet size is comprised between 1 and 100 microns, by admixing the oil phase and the water phase;
4) performing a curing step to form the wall of said microcapsule; and
5) optionally drying the final dispersion to obtain the dried core-shell microcapsule.

In a particular embodiment, the core-shell microcapsule is a formaldehyde-free capsule. A typical process for the preparation of aminoplast formaldehyde-free microcapsules slurry comprises the steps of
1) preparing an oligomeric composition comprising the reaction product of, or obtainable by reacting together:
   a. a polyamine component in the form of melamine or of a mixture of melamine and at least one C₁-C₄ compound comprising two NH₂ functional groups;
   b. an aldehyde component in the form of a mixture of glyoxal, a C₄₋₆ 2,2-dialkoxy-ethanal and optionally a glyoxalate, said mixture having a molar ratio glyoxal/C₄₋₆ 2,2-dialkoxy-ethanal comprised between 1/1 and 10/1; and
   c. a protic acid catalyst;
2) preparing an oil-in-water dispersion, wherein the droplet size is comprised between 1 and 600 microns, and comprising:
   a. an oil;
   b. a water medium:
   c. at least an oligomeric composition as obtained in step 1;
   d. at least a cross-linker selected amongst:
      i. C₄-C₁₂ aromatic or aliphatic di- or tri-isocyanates and their biurets, triurets, trimmers, trimethylol propane-adduct and mixtures thereof, and/or
      ii. a di- or tri-oxiran compounds of formula:

Q-(oxiran-2-ylmethyl)ₙ
wherein n stands for 2 or 3 and Q represents a C₂-C₆ group optionally comprising from 2 to 6 nitrogen and/or oxygen atoms;
e. optionally a C₁-C₄ compounds comprising two NH₂ functional groups;
3) Heating the dispersion; and
4) Cooling the dispersion.

The above process is described in more details in WO 2013/068255.

In a particular embodiment of the core-shell microcapsules, the core-shell microcapsule is a polyamide core-shell polyamide microcapsule comprising:
an oil based core comprising an hydrophobic active, preferably comprising at least one compound of formula (I), and
a polyamide shell comprising or being obtainable from:
   an acyl chloride,
   a first amino compound, and
   a second amino compound.

According to a particular embodiment, the polyamide core-shell microcapsule comprises:
an oil based core comprising an hydrophobic active, preferably comprising at least one compound of formula (I), and
a polyamide shell comprising or being obtainable from:
   an acyl chloride, preferably in an amount comprised between 5 and 98%, preferably between 20 and 98%, more preferably between 30 and 85% w/w
   a first amino compound, preferably in an amount comprised between 1% and 50% w/w, preferably between 7 and 40% w/w;
   a second amino compound, preferably in an amount comprised between 1% and 50% w/w, preferably between 2 and 25% w/w
   a stabilizer, preferably a biopolymer, preferably in an amount comprised between 0 and 90%, preferably between 0.1 and 75%, more preferably between 1 and 70%.

According to a particular embodiment, the polyamide core-shell microcapsule comprises:
an oil based core comprising a hydrophobic active, preferably comprising at least one compound of formula (I), and
a polyamide shell comprising or being obtainable from:
   an acyl chloride,
   a first amino-compound being an amino-acid, preferably chosen in the group consisting of L-Lysine, L-Arginine, L-Histidine, L-Tryptophane and/or mixture thereof.
   a second amino compound chosen in the group consisting of ethylene diamine, diethylene triamine, cystamine and/or mixture thereof, and
   a biopolymer chosen in the group consisting of casein, sodium caseinate, bovin serum albumin, whey protein, and/or mixture thereof.

The first amino-compound can be different from the second amino-compound. Typically, a process for preparing a polyamide-based micrcoapsule includes the following steps:
a) Dissolving at least one acyl chloride in a hydrophobic material, preferably a perfume to form an oil phase;
b) Dispersing the oil phase obtained in step a) into a water phase comprising a first amino compound to form an oil-in water emulsion;
c) Performing a curing step to form polyamide microcapsules in the form of a slurry; wherein a stabilizer is added in the oil phase and/or in the water phase, and wherein at least a second amino-compound is added in the water phase before the formation of the oil-in-water emulsion and/or in the oil-in water emulsion obtained after step b).

In a particular embodiment, the shell of the microcapsule is polyurea- or polyurethane-based. Examples of processes for the preparation of polyurea and polyureathane-based microcapsule slurry are for instance described in WO 2007/004166, EP 2300146, and EP 2579976. Typically a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a water phase;
c) Adding the oil phase to the water phase to form an oil-in-water dispersion, wherein the mean droplet size is comprised between 1 and 500 μm, preferably between 5 and 50 m; and
d) Applying conditions sufficient to induce interfacial polymerisation and form microcapsules in form of a slurry.

In a particular embodiment, the microcapsule can be in form of a powder, which in particular may be obtained by submitting the microcapsule slurry to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, gum Arabic, vegetable gums, pectins, xanthans, alginates, carrageenans or cellulose derivatives to provide microcapsules in a powder form.

However, one may cite also other drying method such as the extrusion, plating, spray granulation, the fluidized bed, or even a drying at room temperature using materials (carrier, desiccant) that meet specific criteria as disclosed in WO 2017/134179.

In another aspect, the present invention relates to a method to confer, enhance, improve or modify the odor properties of a perfuming composition, the air surrounding the perfuming composition, a surface or a perfumed article, comprising adding to the composition, the air, or article, or contacting or treating the surface with an effective amount of at least one compound of formula (I) as defined above. The term “surface”, as used herein may refer to a user's skin, hair, a textile, or hard surface, on to which, a perfume composition comprising or containing the at least one compound of formula (I) is applied.

In another aspect, the present invention relates to a method for intensifying or prolonging the diffusion effect of the characteristic fragrance of at least one aldehyde compound of formula (II), of at least one formate ester of formula (III) and/or of at least one alcohol of formula (IV) as defined above, on a surface or the air surrounding the perfuming composition, wherein the surface, or the air is treated with at least one compound (I) as defined above, or with a composition or article containing at least one compound (I), under conditions susceptible of allowing the release of at least one aldehyde compound formula (II), of at least one formate ester of formula (III) and/or of at least one alcohol of formula (IV) over time.

Moreover, the present invention relates to a perfuming composition comprising
i) at least one compound of formula (I), as defined above;
ii) at least one ingredient selected from the group consisting of a perfumery carrier and a perfumery base; and
iii) optionally at least one perfumery adjuvant.

By “perfumery carrier” it is meant here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, Abalyn® (rosin resins, available from Eastman), benzyl benzoate, benzyl alcohol, 2-(2-ethoxyethoxy)-1-ethano, tri-ethyl citrate or mixtures thereof, which are the most commonly used or also naturally derived solvents like glycerol or various vegetable oils such as palm oil, sunflower oil or linseed oil. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. The use of solid carrier is of current use in the art and a person skilled in the art knows how to reach the desired effect. However by way of non-limiting example of solid carriers, one may cite absorbing gums or polymers or inorganic material, such as porous polymers, cyclodextrins, dextrins, maltodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, tri- and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualitat, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation technique.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycouryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cy Mel® (origin: Cytec Technology Corp.), Urecoll® or Luracoll® (origin: BASF).

Others resins one are the ones produced by the polycondensation of an a polyol, like glycerol, and a polyisocyanate, like a trimer of hexamethylene diisocyanate, a trimer of isophorone diisocyanate or xylene diisocyanate or a Biuret of hexamethylene diisocyanate or a trimer of xylene diisocyanate with trimethylolpropane (known with the tradename of Takenate®, origin: Mitsui Chemicals), among which a trimer of xylene diisocyanate with trimethylolpropane and a Biuret of hexamethylene diisocyanate.

Some of the seminal literature related to the encapsulation of perfumes by polycondensation of amino resins, namely melamine based resins with aldehydes includes represented by articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinency, which disclose suitable uses of such microcapsules, are represented for example by the article of H. Y. Lee et al. Journal of Microencapsulation, 2002, vol. 19, pages 559-569, international patent publication WO 01/41915 or yet the article of S. Bône et al. Chimia, 2011, vol. 65, pages 177-181.

The term “perfumery base” is understood as a composition comprising at least one perfuming co-ingredient.

The perfuming co-ingredient is not a compound according to the invention. Moreover, the term “perfuming co-ingredient” is understood as a compound, which is used in a perfuming preparation or composition to impart a hedonic effect; i.e. used for the primary purpose of conferring or modulating an odor. In other words, such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of general knowledge and according to intended use or application and the desired organoleptic effect. In general terms, these perfuming co-ingredients belong to chemical classes as varied as alcohols, lactones, aldehydes, ketones, esters, ethers, acetates, nitriles, terpene hydrocarbons, nitrogenous or sulphurous heterocyclic compounds and essential oils, and the perfuming co-ingredients can be of natural or synthetic origin.

In particular one may cite perfuming co-ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0^{2,7}]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Baalsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 4,6,6,7,8,8-hexamethyl-1,3,4,6,7,8-hexahydrocyclopenta[g]isochromene, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0²′7]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood® (Origin: Firmenich SA), (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

A perfumery base according to the invention may not be limited to the above mentioned perfuming co-ingredients, and many other of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds also known as properfume or profragrance.

Non-limiting examples of suitable properfumes or profragrances may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 3-(dodecylsulfonyl)-1-(2,6,6-trimethylcyclohex-3-en-1-yl)butan-1-one, a linear polysiloxane co-polymer of 3-((3-(dimethoxy(methyl)silyl)propyl)thio)-2-methyl-5-(prop-1-en-2-yl)cyclohexan-1-one, 3-(dodecylthio)-1-(6-ethyl-2,6-dimethylcyclohex-3-en-1-yl)butan-1-one, 2-(dodecylthio)octan-4-one, 2-(dodecylsulfonyl)octan-4-one, 4-oxooctan-2-yl dodecanoate, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2E,6Z)-2,6-nonadienyl hexadecanoate, (2E,6Z)-2,6-nonadien-1-yl tetradecanoate, (2E,6Z)-2,6-nonadien-1-yl dodecanoate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, (2-((2-methyl-4-(2,6,6-trimethylcyclohex-1-en-1-yl)but-1-en-1-yl)oxy)ethyl)benzene, 1-methoxy-4-(2-methyl-3-phenethoxyallyl)benzene, (2-((2-isopropyl-5-methylcyclohexylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 2-ethoxy-1-((2-methoxy-2-phenylvinyl)oxy)-4-methylbenzene, 3-methoxy-4-((2-methoxy-2-phenylvinyl)oxy)benzaldehyde, 1-isopropyl-2-((2-methoxy-2-phenylvinyl)oxy)-4-methylbenzene, 4-((2-(hexyloxy)-2-phenylvinyl)oxy)-3-methoxybenzaldehyde or a mixture thereof.

The term “perfumery adjuvant” is understood as an ingredient capable of imparting additional added benefit such as a color, a particular light resistance, chemical stability and etc. A detailed description of the nature and type of adjuvant commonly used in perfuming bases cannot be exhaustive, but it has to be mentioned that the ingredients are well known to a person skilled in the art. However, one may cite as specific non-limiting examples the following: viscosity agents (e.g. surfactants, thickeners, gelling and/or rheology modifiers), stabilizing agents (e.g. preservatives, antioxidants, heat/light and or buffers or chelating agents, such as BHT), coloring agents (e.g. dyes and/or pigments), preservatives (e.g. antibacterial or antimicrobial or antifungal or anti-irritant agents), abrasives, skin cooling agents, fixatives, insect repellants, ointments, vitamins and mixture thereof. By “fixative” also called “modulator”, it is understood here an agent having the capacity to affect the manner in which the odour, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Non-limiting examples of suitable modulators may include methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and a combination thereof. At most 20% by weight, based on the total weight of the perfuming composition, of the modulator may be incorporated into the perfumed consumer product.

It is understood that a person skilled in the art is perfectly able to design optimal formulations for the desired effect by admixing the above-mentioned components of a perfuming composition, simply by applying the standard knowledge of the art as well as by trial and error methodologies.

An invention's composition consisting of at least one of the invention's compounds of formula (I) and at least one perfumery carrier represents a particular embodiment of the invention as well as a perfuming composition comprising at least one of the invention's compounds of formula (I), at least one perfumery carrier, at least one perfumery base, and optionally at least one perfumery adjuvant.

It is useful to mention here that the possibility to have, in the compositions mentioned above, more than one of the invention's compounds of formula (I) or other precursors of similar type is important as it enables the perfumer to prepare accords, perfumes, possessing the odor tonality of various compounds of the invention, creating thus new building block for creation purposes.

For the sake of clarity, it is also understood that any mixture resulting directly from a chemical synthesis, e.g. a reaction medium without an adequate purification, in which the compound of the invention would be involved as a starting, intermediate or end-product could not be considered as a perfuming composition according to the invention as far as the mixture does not provide the inventive compound in a suitable form for perfumery. Thus, unpurified reaction mixtures are generally excluded from the present invention unless otherwise specified.

Furthermore, the invention's compounds of formula (I) can also be advantageously used in all the fields of modern perfumery, i.e. fine or functional perfumery, to positively impart or modify the odor of a consumer product into which the compound (I) is added. Therefore, the present invention also relates to a perfumed consumer product comprising at least one compound of formula (I), as defined above or a perfuming composition as defined above.

For the sake of clarity, it has to be mentioned that, the term “perfumed consumer product” is understood as a consumer product which is expected to deliver at least a pleasant perfuming effect to the surface to which it is applied (e.g. skin, hair, textile, or hard surface). In other words, a perfumed consumer product according to the invention is a perfumed consumer product which comprises the functional formulation, as well as optionally additional benefit agents, corresponding to the desired consumer product, e.g. a conditioner, a detergent or an air freshener, and an olfactively effective amount of at least one invention's compound. For the sake of clarity, the perfuming consumer product is a non-edible product.

The nature and type of the constituents of the perfuming consumer product do not warrant a more detailed description here, which in any case would not be exhaustive, the skilled person being able to select them on the basis of his general knowledge and according to the nature and the desired effect of the product.

In one embodiment, the perfumed consumer product is a perfume, a fabric care product, a body-care product, a cosmetic preparation, a skin-care product, an air care product or a home care product.

Non-limiting examples of suitable perfumed consumer products include a perfume, such as a fine perfume, a splash or eau de parfum, a cologne or a shave or after-shave lotion; a fabric care product, such as a liquid or solid detergent optionally in the form of a pod or or tablet, a fabric softener, a liquid or solid scent booster, a dryer sheet, a fabric refresher, an ironing water, a paper, a bleach, a carpet cleaner, a curtain-care product; a body-care product, such as a hair care product (e.g. a shampoo, a leave-on or rinse-off hair conditioner, a coloring preparation or a hair spray, a color-care product, a hair shaping product), a dental care product, a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup; or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furniture care product, a wipe, a dish detergent or a hard-surface (e.g. a floor, bath, sanitary or a window-cleaning) detergent; a leather care product; a car care product, such as a polish, a wax or a plastic cleaner.

Typical examples of fabric detergents or softener compositions into which the compounds of the invention can be incorporated are described in WO 97/34986 or in U.S. Pat. Nos. 4,137,180 and 5,236,615 or EP 799 885. Other typical detergent and softening compositions which can be used are described in works such as Ullmann's Encyclopedia of Industrial Chemistry, Vol. 20, Wiley-VCH, Weinheim, p. 355-540 (2012); Flick, Advanced Cleaning Product Formulations, Noye Publication, Park Ridge, New Jersey (1989); Showell, in Surfactant Science Series, vol. 71: Powdered Detergents, Marcel Dekker, New York (1988); Proceedings of the World Conference on Detergents (4th, 1998, Montreux, Switzerland), AOCS print.

According to any embodiments of the invention, the perfumed consumer product of the invention are characterized by a pH of 1 or more. Particularly, the perfumed consumer product of the invention has a pH comprised between 1 and 12, or between 1 and 8. Even more particularly, the perfumed consumer product of the invention has a pH comprised between 1 and 6.

According to a particular embodiment, the invention's perfumed consumer product is in the form of a personal care, a home care or fabric care consumer product comprising ingredients that are common in the personal, home or fabric care consumer products, in particular shower gel, shampoo, soap, fabric detergents or softeners and all-purpose cleaners. The main functional constituents of perfumed consumer products are surfactants and/or softener components capable of cleaning and/or softening fabrics and/or textiles of varied nature, such as clothes, curtain fabrics, carpet and furniture fabrics, etc, or other home surfaces, skin or hair, and typically used in a large amount of water or water-based solvents. These are therefore formulations wherein the amount of water is typically comprised between 50 and 99% by weight of the perfumed consumer product with the exception of soap or solid detergent wherein the amount of water is at most 20%.

A more detailed description of such fabric cleaning and/or softening formulations is not warranted here, many descriptions of current liquid formulations can be found in the cleaner/fabric softener's patent and other pertinent literature, such as for example the textbook of Louis Ho Tan Tai, “Détergents et Produits de Soins Corporels, Chapters 1 to 7 in particular, Dunod, Paris, 1999, or any other similar and/or more recent textbooks pertaining to the art of liquid softener and all-purpose cleaners formulations. A patent publication, WO 2010/105873, is also cited by way of example, in as much as it describes typical current ingredients, other than perfumes, of such liquid products, particularly in pages 9 to 21. Of course, many other examples of liquid cleaner and/or fabric softener formulations can be found in the literature. Any such liquid formulations, namely liquid fabric cleaner or conditioner and/or all-purpose cleaner, can be used in the here-described compositions.

According to a particular embodiment of the invention, the invention's perfumed consumer product is a liquid fabric softener comprising a fabric softener active base in amount comprised between 85 and 100% by weight, based on the total weight of the perfumed consumer product. The main constituent of the fabric softener active base is water or water-based solvents. The fabric softener active base may comprise dialkyl quaternary ammonium salts, dialkyl ester quaternary ammonium salts, Hamburg esterquat, triethanolamine quat, silicones and mixtures thereof. Optionally, the fabric softener active base of the composition may further comprise a viscosity modifier in an amount comprised between 0.05 and 1% by weight, based on the total weight of the liquid base; preferably chosen in the group consisting of calcium chloride.

According to a particular embodiment of the invention, the invention's perfumed consumer product is an all-purpose cleaner comprising an all-purpose cleaner active base in amount comprised between 85 and 100% by weight, based on the total weight of the perfumed consumer product. The main constituent of the all-purpose cleaner active base is water or water-based solvents The all-purpose active base may comprise linear alkylbenzene sulfonates (LAS) in an amount comprised between 1 and 2%, nonionic surfactant in an amount comprised between 2 and 4% and acid such as citric acid in an amount comprised between 0.1 and 0.5%.

According to a particular embodiment of the invention, the invention's perfumed consumer product is a liquid detergent comprising a liquid detergent active base in amount comprised between 85 and 100% by weight, based on the total weight of the perfumed consumer product. The main constituent of the liquid detergent active base is water or water-based solvents. The liquid detergent active base may comprise anionic surfactant such as alkylbenzenesulfonate (ABS), linear alkylbenzene sulfonates (LAS), secondary alkyl sulfonate (SAS), primary alcohol sulfate (PAS), lauryl ether sulfate (LES), sodium lauryl ether sulfate (SLES), methyl ester sulfonate (MES); nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides; ormixtures thereof.

According to a particular embodiment of the invention, the invention's perfumed consumer product is a solid detergent comprising a solid detergent active base in amount comprised between 85 and 100% by weight, based on the total weight of the perfumed consumer product. The solid detergent active base may comprise at least one surfactant chosen in the group consisting of anionic, nonionic, cationic, zwiterionic surfactant and mixtures thereof. The surfactant in the solid detergent active base is preferably chosen in the group consisting of linear alkene benzene sulphonate (LABS), sodium laureth sulphate, sodium lauryl ether sulphate (SLES), sodium lauryl sulphate (SLS), alpha olefin sulphonate (AOS), methyl ester sulphonates (MES), alkyl polyglyucosides (APG), primary alcohol ethoxylates and in particular lauryl alcohol ethoxylates (LAE), primary alcohol sulphonates (PAS), soap and mixtures thereof. The soild detergent active base may comprise a further component, commonly used in powder detergent consumer product, selected from the group consisting of bleaching agents such as TAED (tetraacetylethylenediamine); buffering agent; builders such as zeolites, sodium carbonate or mixture thereof; soil release or soil suspension polymers; granulated enzyme particles such as cellulase, lipase, protease, mannanase, pectinase or mixtures thereof; corrosion inhibitor; antifoaming; sud suppressing agents; dyes; fillers such as sodium silicate, sodium sulfate or mixture thereof; source of hydrogen peroxide such as sodium percarbonate or sodium perborate; and mixtures thereof.

According to a particular embodiment of the invention, the invention's perfumed consumer product is shampoo or a shower gel comprising a shampoo or shower gel active base in amount comprised between 85 and 100% by weight, based on the total weight of the perfumed consumer product. The main constituent of the shampoo or a shower gel active base is water or water-based solvents The shampoo shower gel active base may comprise sodium alkylether sulfate, ammonium alkylether sulfates, alkylamphoacetate, cocamidopropyl betaine, cocamide MEA, alkylglucosides and aminoacid based surfactants.

According to a particular embodiment of the invention, the invention's perfumed consumer product is a soap bar comprising a soap active base in amount comprised between 85 and 100% by weight, based on the total weight of the perfumed consumer product. The soap bar active base may comprise salt of a weak acid, typically, a salt of weak acid, which may be a fatty acid and strong base like sodium hydroxide.

The proportions in which the compounds according to the invention can be incorporated into the various aforementioned articles or compositions vary within a wide range of values. These values are dependent upon the nature of the article or product to be perfumed and on the desired olfactory effect as well as the nature of the co-ingredients in a given composition when the compounds according to the invention are mixed with perfuming co-ingredients, solvents or additives commonly used in the art.

For example, in the case of perfuming compositions, typical concentrations are in the order of 0.001% to 10% by weight, or even more, of the compounds of the invention based on the weight of the composition into which they are incorporated. In the case of perfumed consumer product, typical concentrations are in the order of 0.0001% to 5% by weight, or even more, of the compounds of the invention based on the weight of the consumer product into which they are incorporated.

Moreover, the present invention relates to a compound of formula (I). So another object of the invention is a compound of formula

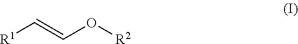

in the form of any one of its stereoisomers or a mixture thereof and wherein
R¹ comprises at least 6 carbon atoms and is a C₁₋₁₅ alkyl, C₃₋₁₅ alkenyl, C₃₋₁₅ cycloalkyl, C₅₋₁₅ is cycloalkenyl or C₃₋₁₄ heterocycloalkyl group, each optionally substituted with one or more of a hydroxy, C₁₋₁₅ alkyl, C₂₋₁₅ alkenyl, C₁₋₁₅ alkoxy, C₂₋₁₅ alkenyloxy, C₃₋₁₅ cycloalkyl, C₅₋₁₅ cycloalkenyl, C₃₋₁₅ heterocycloalkyl, carboxylic acid, C₁₋₄ carboxylic ester, C₆₋₁₀ aryl and/or C₆₋₁₀ aryloxy group, each optionally substituted with one or more of a C₁₋₈ alkyl, C₁₋₈ alkoxy, hydroxy, carboxylic acid and/or C₁₋₄ carboxylic ester group, wherein the heteroatom represents one or more of an oxygen atom, provided that R¹ is not a 2-hexylidenecyclopentyl group;
R² a C₆₋₈ hydrocarbon group optionally comprising one or two oxygen atoms, provided that an ester functional group alpha to the oxy group is excluded; and provided that R² is not a benzyl group or a cyclohexyl group or 2-hydroxy-1,2-diphenylethyl, or a 1-(tert-butoxy)-7,7-dimethylbicyclo[2.2.1]heptan-2-yl group and does not comprise an allylic functional group; and provided that 1-(heptyloxy)dec-1-ene, 1-(decyloxy)dec-1-ene, 1-(dodecyloxy)dodec-1-ene, (4-phenethoxybut-3-en-1-yl)benzene and 1-((3,7-dimethyloctyl)oxy)-3,7-dimethyloct-1-ene are excluded.

The terms “benzyl group” is understood as a CH₂C₆H₄ group; i.e. the phenyl group is unsubstituted.

The terms “cyclohexyl group” is understood as a C₆H₁₁ group; i.e. the cyclohexyl group is unsubstituted.

In a further aspect, the present invention also relates to the use of precursor compounds for releasing compounds selected from the group consisting of
a) an aldehyde compound of formula

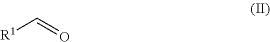

wherein
R¹ is a C₁₋₁₅ alkyl, C₃₋₁₅ alkenyl, C₃₋₁₅ cycloalkyl, C₅₋₁₅ cycloalkenyl or C₃₋₁₄ heterocycloalkyl group, each optionally substituted with one or more of a hydroxy, C₁₋₁₅ alkyl, C₂₋₁₅ alkenyl, C₁₋₁₅ alkoxy, C₂₋₁₅ alkenyloxy, C₃₋₁₅ cycloalkyl, C₅₋₁₅ cycloalkenyl, C₃₋₁₅ heterocycloalkyl, carboxylic acid, C₁₋₄ carboxylic ester, C₆₋₁₀ aryl and/or C₆₋₁₀ aryloxy group, each optionally substituted with one or more of a C₁₋₈ alkyl, C₁₋₈ alkoxy, hydroxy, carboxylic acid and/or C₁₋₄ carboxylic ester group;
b) a formate ester of formula

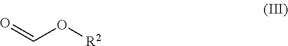

R² is a C₁₋₁₈ hydrocarbon group optionally comprising one, two or three oxygen atoms; provided that an ester functional group alpha to the formyloxy group is excluded;
c) an alcohol of formula

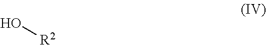

wherein R² has the same meaning as defined above;
wherein the precursor compound comprises a compound of formula (I)

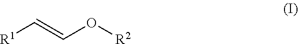

in the form of any one of its stereoisomers or a mixture thereof, and wherein R¹ and R² have the same meaning as defined above;
   by exposing the precursor compound of formula (I) to an environment wherein the compound is oxidized.

In a further aspect, the present invention relates to the use of at least one compound of formula (I) as defined above to confer, enhance, improve or modify the odor properties of a perfuming composition, the air surrounding the perfuming composition, a surface, or of a perfumed article, comprising adding to the composition or article or contacting or treating the surface with an effective amount of at least one compound of formula (I) as defined above. The term “surface”, as used herein may refer to a user's skin, hair, a textile, or hard surface, on to which, a perfume composition comprising or containing the at least one compound of formula (I) is applied.

In a further aspect, the present invention relates to the use of at least one compound of formula (I) as defined above for intensifying or prolonging the diffusion effect, and/or perception of the characteristic fragrance of at least one aldehyde compound formula (II), of at least one formate ester of formula (III) and/or of at least one alcohol of formula (IV) as defined above, on a surface, wherein the surface is treated with at least one compound of formula (I) as defined above, or with a composition or article containing the at least one compound of formula (I), under conditions susceptible of allowing the release of the at least one aldehyde compound formula (II), of at least one formate ester of formula (III) and/or of at least one active alcohol of formula (IV) over time.

The compounds of formula (I) can be prepared according to standard methods known in the art as described herein-below.

### EXAMPLES

The invention will now be described in further detail by way of the following examples, wherein the abbreviations have the usual meaning in the art, the temperatures are indicated in degrees centigrade (° C.). NMR spectra were acquired using either a Bruker Avance II Ultrashield 400 plus operating at 400 MHz, (H) and 100 MHz (¹³C) or a Bruker Avance III 500 operating at 500 MHz (H) and 125.8 MHz (¹³C) or a Bruker Avance III 600 cryoprobe operating at 600 MHz (H) and 151 MHz (¹³C). Spectra were internally referenced relative to tetramethyl silane 0.0 ppm. ¹H NMR signal shifts are expressed in 5 ppm, coupling constants (J) are expressed in Hz with the following multiplicities: s, singlet; d, doublet; t, triplet; q, quartet; m, multiplet; b, broad (indicating unresolved couplings) and were interpreted using Bruker Topspin software. ¹³C NMR data are expressed in chemical shift 3 ppm and hybridization from DEPT 90 and DEPT 135 experiments, C, quaternary; CH, methine; CH₂, methylene; CH₃, methyl.

### Example 1

### Preparation of Compounds According to Formula (I) and Comparative Compounds

Compound 1. (2-(dodec-1-en-1-yloxy)ethyl)benzene: The dimethyl acetal of dodecanal (13 g, 56.4 mmol), 2-phenylethanol (17.9 g, 146 mmol) and KHSO₄ (0.037 g, 0.27 mmol) were added to a 35 ml, round-bottomed flask equipped with a distillation head and nitrogen bubbler. The mixture was heated at 150° C. (oil bath) while distilling out the liberated alcohol for 1 h. The mixture was allowed to cool then placed under vacuum (4 Pa) and heated (190° C. oil bath) while allowing liberated phenylethanol and the enol ether to distill from the reaction flask. Fractions rich in the enol ether were combined and, after adding Na₂CO₃ (0.3 g), the distillate was subjected to short-path distillation (bp 140° C., 4 Pa) to afford 6.9 g (23.9 mmol, 42% yield) of the title compound as a colorless oil (E/Z=30:70).

¹H NMR (CDCl₃, 600 MHz): δ 0.877_{E} and 0.881_{Z} (both t, J=7.0 Hz, 3H), 1.20-1.36 (m, 16H), 1.89_{E} (q, J=7.1 Hz, 0.6H), 2.05_{Z} (q, J=7.1 Hz, 1.4H), 2.91_{Z} (t, J=7.2 Hz, 1.4H), 2.94_{E} (t, J=7.2 Hz, 0.6H), 3.84_{E} (t, J=7.2 Hz, 0.6H), 3.90_{Z} (t, J=7.2 Hz, 1.4H), 4.35_{Z} (q, J=7.1 Hz, 0.7H), 4.77_{E} (dt, J=12.6, 7.3 Hz, 0.3H), 5.92_{Z} (d, J=6.3 Hz, 0.7H), 6.22_{E} (d, J=12.6 Hz, 0.3), 7.18-7.24 (m, 3H), 7.26-7.31 (m, 2H).

¹³C NMR (CDCl₃, 151 MHz): δ 14.14 (CH₃), 22.71 (CH₂), 22.72 (CH₂), 24.02 (CH₂), 27.77 (CH₂), 29.05 (CH₂), 29.33 (CH₂), 29.37 (CH₂), 29.40 (CH₂), 29.52 (CH₂), 29.56 (CH₂), 29.66 (CH₂), 29.67 (CH₂), 29.69 (CH₂), 29.71 (CH₂), 29.83 (CH₂), 30.74 (CH₂), 31.94 (CH₂), 31.96 (CH₂), 35.83 (CH₂), 36.39 (CH₂), 69.68 (CH₂), 72.75 (CH₂), 104.59 (CH), 107.60 (CH), 126.33 (CH), 126.36 (CH), 128.38 (CH), 128.42 (CH), 128.92 (CH), 129.01 (CH), 138.32 (C), 138.41 (C), 144.45 (CH), 145.81 (CH).

Compound 2. (2-(tridec-1-en-1-yloxy)ethyl)benzene. The dimethyl acetal of tridecnal (10 g, 40.9 mmol), 2-phenylethanol (15.0 g, 123 mmol), and KHSO₄ (0.17 g, 1.24 mmol) were added to a 35 ml, round-bottomed flask equipped with a distillation head and nitrogen bubbler. The mixture was heated at 140° C. (oil bath) while allowing liberated methanol to distill from the reaction vessel. After 1 h, the reaction vessel was placed under vacuum (60 kPa) and the pressure progressively reduced to 1.3 Pa over a 3-h period while allowing 2-phenylethanol to distill from the flask. Following this the title compound (3.28 g, 26.5% yield) was isolated by short-path, vacuum distillation from the reaction flask (bp 155° C., 1 Pa) as a pale-yellow oil (E/Z=38:62).

¹H NMR (CDCl₃, 500 MHz): δ 0.88 (t, J=7.0 Hz, 3H), 1.20-1.36 (m, 18H), 1.89_{E} (q, J=7.0 Hz, 0.76H), 2.05_{Z} (q, J=7.1 Hz, 1.24H), 2.92_{Z} (t, J=7.2 Hz, 1.24H), 2.94_{E} (t, J=7.2 Hz, 0.76H), 3.84_{E} (t, J=7.2 Hz, 0.76H), 3.90_{Z} (t, J=7.2 Hz, 1.24H), 4.35_{Z} (q, J=7.1 Hz, 0.38H), 4.77_{E} (dt, J=12.6, 7.3 Hz, 0.62H), 5.92_{Z} (d, J=6.3 Hz, 0.62H), 6.22_{E} (d, J=12.6 Hz, 0.38H), 7.18-7.24 (m, 3H), 7.26-7.31 (m, 2H).

¹³C NMR (CDCl₃, 125.8 MHz): δ 14.13 (CH₃), 22.71 (CH₂), 24.01 (CH₂), 27.77 (CH₂), 29.05 (CH₂), 29.32 (CH₂), 29.37 (CH₂), 29.39 (CH₂), 29.52 (CH₂), 29.56 (CH₂), 29.66 (CH₂), 29.69 (CH₂), 29.70 (CH₂), 29.73 (CH₂), 29.82 (CH₂), 30.73 (CH₂), 31.94 (CH₂), 31.95 (CH₂), 35.84 (CH₂), 36.39 (CH₂), 69.69 (CH₂), 72.75 (CH₂), 104.61 (CH), 107.61 (CH), 126.33 (CH), 126.37 (CH), 128.38 (CH), 128.43 (CH), 128.93 (CH), 129.02 (CH), 138.32 (C), 138.41 (C), 144.45 (CH), 145.81 (CH).

### Compound 3. (2-(undeca-1,10-dien-1-yloxy)ethyl)benzene

General Procedure: A mixture of the aldehyde (120 mmol), 2-phenylethanol (4 equiv), TsOH (0.025 equiv) and toluene (100 mL) was heated at reflux for 1 h while removing water with a Dean-Stark trap. After the mixture cooled, it was diluted with diethyl ether and washed with sat. Na₂CO₃ and water. The organic phase was dried with Na₂SO₄, filtered and concentrated. Using a Kugelrohr distillation apparatus, excess phenylethanol was removed by heating the product under vacuum (oven at 140° C., 3.3 Pa). The resulting phenylethyl acetals (40-80 mmol) and KHSO₄ (0.059 g, 0.40-0.80 mmol) were charged to a 35-ml, round-bottomed flask fitted with a distillation head. The mixture was placed under vacuum (4 Pa) and then set in a 120° C. oil bath. The bath was heated to 190° C. while allowing liberated phenylethanol and the enol ether to distill from the flask as they formed. Factions rich in the enol ether were combined. After adding Na₂CO₃ (0.5 g), the distillate was subjected to short-path distillation to afford the enol ether as a colorless oil.

Following this general procedure and using the phenylethyl acetal of 10-undecenal (17.1 g, 43.4 mmol), the title compound was isolated by short-path distillation (bp 137-143° C., 4 Pa) in 70% yield (E/Z=35:65).

¹H NMR (CDCl₃, 600 MHz): δ 1.22-1.42 (m, 10H), 1.89_{E} (q, J=7.0 Hz, 0.70H), 2.00-2.08 (m, 3.30H), 2.92_{Z} (t, J=7.1 Hz, 1.30H), 2.94_{E} (t, J=7.1 Hz, 0.70H), 3.84_{E} (t, J=7.2 Hz, 0.70H), 3.90_{Z} (t, J=7.2 Hz, 1.30H), 4.35_{Z} (q, J=6.9 Hz, 0.65H), 4.77_{E} (dt, J=12.6, 7.4 Hz, 0.35H), 4.93 (d, J=10.1 Hz, 1H), 4.99 (d, J=17.1 Hz, 1H), 5.76-5.86 (m, 1H), 5.92_{Z} (d, J=6.2 Hz, 0.65H), 6.22_{E} (d, J=12.6 Hz, 0.35H), 7.19-7.24 (m, 3H), 7.27-7.31 (m, 2H).

¹³C NMR (CDCl₃, 151 MHz): δ 23.99 (CH₂), 27.74 (CH₂), 28.93 (CH₂), 28.96 (CH₂), 29.11 (CH₂), 29.15 (CH₂), 29.23 (CH₂), 29.32 (CH₂), 29.35 (CH₂), 29.76 (CH₂), 30.69 (CH₂), 33.81 (CH₂), 33.84 (CH₂), 35.83 (CH₂), 36.38 (CH₂), 69.70 (CH₂), 72.75 (CH₂), 104.56 (CH), 107.55 (CH), 114.09 (CH₂), 114.12 (CH₂), 126.33 (CH), 126.37 (CH), 128.38 (CH), 128.43 (CH), 128.92 (CH), 129.01 (CH), 138.31 (C), 138.40 (C), 139.22 (CH), 139.27 (CH), 144.48 (CH), 145.83 (CH).

Compound 4. ((3-phenethoxyallyl)benzene): Following the procedure described for Compound 3 and using the phenylethyl acetal of 3-phenylpropanal (28.7 g, 79.6 mmol), the title compound was isolated by bulb-to-bulb distillation using a Kugelrohr distillation apparatus (oven 140-170° C., 4 Pa) in 41% yield (E/Z=35:65).

¹H NMR (CDCl₃, 500 MHz): δ 2.95 (t, J=7.0 Hz, 2H), 3.24_{E} (d, J=7.5 Hz, 0.70H), 3.41_{Z} (d, J=7.5 Hz, 1.30H), 3.87_{E} (t, J=7.1 Hz, 0.70H), 3.97_{Z} (t, J=7.1 Hz, 1.30H), 4.57_{Z} (dt, J=6.1, 7.5 Hz, 0.65H), 4.93_{E} (dt, J=12.6, 7.5 Hz, 0.35H), 6.05_{Z} (d, J=6.1 Hz, 0.65H), 6.34_{E} (d, J=12.6 Hz, 0.35H), 7.13-7.32 (m, 10H).

¹³C NMR (CDCl₃, 125.8 MHz): δ 30.28 (CH₂), 34.06 (CH₂), 35.77 (CH₂), 36.39 (CH₂), 69.71 (CH₂), 72.90 (CH₂), 103.11 (CH), 105.83 (CH), 125.65 (CH), 125.92 (CH), 126.39 (CH), 126.40 (CH), 128.25 (CH), 128.28 (CH), 128.29 (CH), 128.33 (CH), 128.42 (CH), 128.43 (CH), 128.91 (CH), 129.02 (CH), 138.23 (C), 138.29 (C), 141.57 (C), 141.78 (C), 145.19 (CH), 147.03 (CH).

Compound 5. (4-phenethoxybut-3-en-2-yl)benzene: Following the procedure described for Compound 3 and using the phenylethyl acetal of 3-phenylbutanal (15 g, 40.1 mmol), the reaction mixture was heated at 155° C. under vacuum (6.7 Pa) for 1.5 h. It then was diluted with diethyl ether and washed with sat. Na₂CO₃ and water. The organic phase was dried over Na₂SO₄, filtered and concentrated. The title compound (2.8 g, 28% yield) was isolated by bulb-to-bulb distillation using a Kugelrohr distillation apparatus (oven 145-150° C., 2.7 Pa) as a colorless oil (E/Z=52:48).

¹H NMR (CDCl₃, 500 MHz): δ 1.31 and 1.33 (both d, J=7.1 Hz, 3H), 2.91 and 2.93 (both t, J=7.1 Hz, 2H), 3.38 (pentet, J=7.2 Hz, 0.5H), 3.85 (t, J=7.2 Hz, 1H), 3.88-3.98 (m, 1.5H), 4.53_{Z} (dd, J=9.4, 6.2 Hz, 0.5H), 4.97_{E} (dd, J=12.7, 7.7 Hz, 0.5H), 5.93_{Z} (d, J=6.2 Hz, 0.5H), 6.28_{E} (d, J=12.7 Hz, 0.5H), 7.12-7.32 (m, 10H).

¹³C NMR (CDCl₃, 125.8 MHz): δ 22.11 (CH₃), 22.61 (CH₃), 34.45 (CH), 35.82 (CH₂), 36.37 (CH₂), 38.47 (CH), 69.71 (CH₂), 72.88 (CH₂), 109.83 (CH), 112.67 (CH), 125.68 (CH), 125.93 (CH), 126.35 (CH), 126.39 (CH), 126.82 (CH), 126.93 (CH), 128.26 (CH), 128.34 (CH), 128.38 (CH), 128.42 (CH), 128.93 (CH), 129.02 (CH), 138.27 (C), 138.31 (C), 143.60 (CH), 145.74 (CH), 146.87 (C), 147.17 (C).

Compound 6. (3-methyl-5-phenethoxypent-4-en-1-yl)benzene: Following the procedure described for Compound 3 and using the phenylethyl acetal of 3-methyl-5-phenylpentanal (13 g, 32.3 mmol), the reaction mixture was heated at 155° C. (6.7 Pa) for 2 h. It then was diluted with diethyl ether and washed with sat. Na₂CO₃ and water. The organic phase was dried over Na₂SO₄, filtered and concentrated. The title compound (3.45 g, 38% yield) was isolated by bulb-to-bulb distillation using a Kugelrohr distillation apparatus (oven 165° C., 2.4 Pa) as a colorless oil (E/Z=50:50).

¹H NMR (CDCl₃, 500 MHz): δ 0.98 and 1.00 (both d, J=6.7 Hz, 3H), 1.45-1.56 (m, 1H), 1.56-1.66 (m, 1H), 1.99-2.09 (m, 0.5H), 2.49-2.57 (m, 1H), 2.57-2.71 (m, 1.5H), 2.92 and 2.96 (both t, J=7.1 Hz, 2H), 3.85 and 3.91 (both t, J=7.2 Hz, 2H), 4.21_{Z} (dd, J=9.4, 6.3 Hz, 0.5H), 4.66_{E} (dd, J=12.6, 8.8 Hz, 0.5H), 5.94_{Z} (d, J=6.3 Hz, 0.5H), 6.23_{E} (d, J=12.6 Hz, 0.5H), 7.12-7.33 (m, 10H).

¹³C NMR (CDCl₃, 125.8 MHz): δ 21.40 (CH₃), 22.19 (CH₃), 29.03 (CH), 32.60 (CH), 33.71 (CH₂), 33.96 (CH₂), 35.85 (CH₂), 36.38 (CH₂), 39.64 (CH₂), 39.67 (CH₂), 69.77 (CH₂), 72.79 (CH₂), 110.33 (CH), 113.33 (CH), 125.44 (CH), 125.55 (CH), 126.34 (CH), 126.40 (CH), 128.17 (CH), 128.24 (CH), 128.38 (CH), 128.39 (CH), 128.41 (CH), 128.45 (CH), 128.94 (CH), 129.01 (CH), 138.30 (C), 138.36 (C), 142.81 (C), 143.23 (C), 144.01 (CH), 145.37 (CH).

Compound 7. 1-(octyloxy)dodec-1-ene: A mixture of dodecanal dimethyl acetal (3.75 g, 16.3 mmol), octanol (8.46 g, 65.1 mmol) and KHSO₄ (0.022 g, 0.163 mmol) was heated for 3 h at 60° C. and 5 h at 80° C. under vacuum (12 kPa) using a Kugelrohr distillation apparatus. The mixture then was heated at 200° C. (15.3 Pa) and the generated enol ether and octanol were distilled from the pot as they formed. Distillation fractions rich in the enol ether were combined and subjected to silica gel flash chromatography (hexane/EtOAc, 99.5:0.5) followed by bulb-to-bulb distillation (150° C., 8 Pa) to afford the title compound (1.74 g, 36% yield) as a colorless liquid (E/Z=30:70).

¹H NMR (CDCl₃, 500 MHz): δ 0.879 and 0.882 (both t, J=7.1 Hz, 6H), 1.21-1.40 (m, 26H), 1.56-1.66 (m, 2H), 1.89_{E} (q, J=7.1 Hz, 0.6H), 2.06_{Z} (q, J=7.1 Hz, 1.4H), 3.61_{E} (t, J=6.6 Hz, 0.6H), 3.69_{Z} (t, J=6.6 Hz, 1.4H), 4.31_{Z} (dt, J=6.3, 7.3 Hz, 0.7H), 4.75_{E} (dt, J=12.6, 7.3 Hz, 0.3H), 5.90_{Z} (d, J=6.3 Hz, 0.7H), 6.21_{E} (d, J=12.6 Hz, 0.3).

¹³C NMR (CDCl₃, 125.8 MHz): δ 14.11 (CH₃), 14.13 (CH₃), 22.71 (CH₂), 22.75 (CH₂), 23.89 (CH₂), 25.89 (CH₂), 27.77 (CH₂), 26.05 (CH₂), 27.85 (CH₂), 29.09 (CH₂), 29.29 (CH₂), 29.31 (CH₂), 29.34 (CH₂), 29.41 (CH₂), 29.43 (CH₂), 29.57 (CH₂), 29.59 (CH₂), 29.70 (CH₂), 29.73 (CH₂), 29.75 (CH₂), 29.84 (CH₂), 29.91 (CH₂), 30.84 (CH₂), 31.88 (CH₂), 31.99 (CH₂), 69.23 (CH₂), 72.15 (CH₂), 104.17 (CH), 107.01 (CH), 144.83 (CH), 146.17 (CH).

Compound 8. 1-(tert-butyl)-4-(3-phenethoxyallyl)benzene: The dimethyl acetal of 3-(4-(tert-butyl)phenyl)propanal (28.1 g, 119 mmol), 2-phenylethanol (29.2 g, 239 mmol) and KHSO₄ (0.163 g, 1.2 mmol) were added to a 100 ml, round-bottomed flask equipped with a distillation head and nitrogen bubbler. The mixture was heated at 150° C. (oil bath) for 1 h while distilling out MeOH. The mixture was allowed to cool then placed under vacuum (4 Pa) and heated (240° C. oil bath) while allowing liberated phenylethanol and the enol ether to distill from the reaction flask. Fractions rich in the enol ether were combined and subjected to short-path distillation (bp 152° C., 4 Pa) to afford 12.1 g (41.1 mmol, 34% yield) of the title compound as a colorless oil (E/Z=40:60).

¹H NMR (CDCl₃, 600 MHz): δ 1.30_{E} and 1.31_{Z} (both s, 9H), 2.95 (t, J=7.1 Hz, 2H), 3.22_{E} (d, J=7.4 Hz, 0.80H), 3.39_{Z} (d, J=7.5 Hz, 1.20H), 3.87_{E} (t, J=7.1 Hz, 0.80H), 3.97_{Z} (t, J=7.1 Hz, 1.20H), 4.58_{Z} (dt, J=6.2, 7.5 Hz, 0.60H), 4.93_{E} (dt, J=12.6, 7.4 Hz, 0.40H), 6.05_{Z} (d, J=6.2 Hz, 0.60H), 6.34_{E} (d, J=12.6 Hz, 0.40H), 7.10-7.16 (m, 2H), 7.16-7.25 (m, 3H), 7.25-7.36 (m, 4H).

¹³C NMR (CDCl₃, 151 MHz): δ 29.70 (CH₂), 31.40 (CH₃), 31.42 (CH₃), 33.49 (CH₂), 34.32 (C), 34.34 (C), 35.77 (CH₂), 36.39 (CH₂), 69.64 (CH₂), 72.89 (CH₂), 103.17 (CH), 106.03 (CH), 125.19 (CH), 125.22 (CH), 126.37 (CH), 126.38 (CH), 127.87 (CH), 127.91 (CH), 128.41 (CH), 128.42 (CH), 128.91 (CH), 129.02 (CH), 138.23 (C), 138.30 (C), 138.53 (C), 138.69 (C), 145.07 (CH), 146.89 (CH), 148.42 (C), 148.74 (C).

Compound 9. (2-(undec-1-en-1-yloxy)ethyl)benzene: The dimethyl acetal of undecanal (8.0 g, 37.0 mmol), 2-phenylethanol (11.3 g, 92.4 mmol) and KHSO₄ (0.15 g, 1.10 mmol) were added to a 35 ml, round-bottomed flask equipped with a distillation head and nitrogen bubbler. The mixture was heated at 150° C. (oil bath) for 1.5 h while distilling out MeOH. The mixture was allowed to cool then placed under vacuum (typically 67 Pa) and heated (120° C. oil bath). The vacuum was progressively lowered to allow the excess 2-phenylethanol to distill from the reaction flask. Following this, the bath temperature was increased to 200° C. and the vacuum further lowered to 2 Pa to allow the enol ether to distill from the flask as it formed. Fractions rich in the enol ether were combined and subjected to short-path distillation (bp 152° C., 4 Pa) to afford 5.27 g (19.2 mmol, 52% yield) of the title compound as a colorless oil (E/Z=40:60).

¹H NMR (CDCl₃, 500 MHz): δ 0.877 and 0.881 (both t, J=7.0 Hz, 3H), 1.20-1.37 (m, 14H), 1.89 (q, J=6.9 Hz, 0.8H), 2.05 (q, J=7.0 Hz, 1.2H), 2.91 and 2.94 (overlapping t, J=7.2 Hz, 2H), 3.84 (t, J=7.2 Hz, 0.8H), 3.90 (t, J=7.2 Hz, 1.2H), 4.35 (dt, J=6.2, 7.3 Hz, 0.6H), 4.77 (dt, J=12.6, 7.4 Hz, 0.4H), 5.92 (d, J=6.3 Hz, 0.7H), 6.22 (d, J=12.6 Hz, 0.3H), 7.18-7.24 (m, 3H), 7.26-7.31 (m, 2H).

¹³C NMR (CDCl₃, 125.8 MHz): δ 14.13 (CH₃), 14.14 (CH₃), 22.70 (CH₂), 22.71 (CH₂), 24.0 (CH₂), 27.8 (CH₂), 29.0 (CH₂), 29.3 (CH₂), 29.35 (CH₂), 29.38 (CH₂), 29.51 (CH₂), 29.55 (CH₂), 29.6 (CH₂), 29.7 (CH₂), 29.8 (CH₂), 30.7 (CH₂), 31.91 (CH₂), 31.94 (CH₂), 35.8 (CH₂), 36.4 (CH₂), 69.7 (CH₂), 72.8 (CH₂), 104.6 (CH), 107.6 (CH), 126.3 (CH), 126.4 (CH), 128.38 (CH), 128.43 (CH), 128.9 (CH), 129.0 (CH), 138.3 (C), 138.4 (C), 144.4 (CH), 145.8 (CH).

Compound 10. (2-(undec-1-en-1-yloxy)ethoxy)benzene: Following the procedure described for compound 9 and starting from the dimethyl acetal of undecanal (10.0 g, 46.219 mmol), 2-phenoxyethanol (16.0 g, 115.5 mmol) and KHSO₄ (0.188 g, 1.38 mmol), the title compound (2.2 g, 7.6 mmol) was isolated by distillation (bp 138° C., 2.6 Pa) in 16% yield as a colorless liquid (E/Z=35:65).

¹H NMR (C₆D₆, 600 MHz): δ 0.90 and 0.92 (both t, J=7.0 Hz, 3H), 1.19-1.38 (m, 12.7H), 1.43 (pentet, J=7.5 Hz, 1.3H), 1.89 (q, J=7.1 Hz, 0.7H), 2.31 (q, J=7.3 Hz, 1.3H), 3.62-3.65 (m, 1.3H), 3.65-3.69 (m, 2H), 3.75-3.78 (m, 0.7H), 4.46 (q, J=7.0 Hz, 0.65H), 4.81 (dt, J=12.6, 7.4 Hz, 0.35H), 5.89 (d, J=6.3 Hz, 0.65H), 6.29 (d, J=12.6 Hz, 0.35H), 6.77-6.85 (m, 3H), 7.08-7.13 (m, 2H).

¹³C NMR (C₆D₆, 150.9 MHz): δ 14.4 (CH₃), 23.09 (CH₂), 23.10 (CH₂), 24.5 (CH₂), 28.2 (CH₂), 29.5 (CH₂), 29.7 (CH₂), 29.78 (CH₂), 29.8 (CH₂), 30.0 (CH₂), 30.1 (CH₂), 32.30 (CH₂), 32.31 (CH₂), 66.6 (CH₂), 67.1 (CH₂), 67.5 (CH₂), 70.4 (CH₂), 104.5 (CH), 107.7 (CH), 114.90 (CH), 114.94 (CH), 121.12 (CH), 121.13 (CH), 129.68 (CH), 129.72 (CH), 145.4 (CH), 146.6 (CH), 159.25 (C), 159.30 (C).

Compound 11. 1-((3,7-dimethyloctyl)oxy)undec-1-ene: Following the procedure described for compound 9 and starting from dimethyl acetal of undecanal (10.0 g, 46.2 mmol), 3,7-dimethyloctan-lol (18.3 g, 115.6 mmol) and KHSO₄ (0.25 g, 1.84 mmol), the title compound (8.8 g, 7.6 mmol) was isolated by distillation (bp 140° C., 4 Pa) in 61% yield as a colorless liquid (E/Z=35:65).

¹H NMR (C₆D₆, 600 MHz): δ 0.83 and 0.85 (both d, J=6.7 Hz, 3H), 0.87-0.93 (m, 9H), 1.01-1.09 (m, 1H), 1.09-1.17 (m, 2H), 1.17-1.34 (m, 14H), 1.34-1.43 (m, 2.65H), 1.43-1.64 (m, 4H), 1.64-1.72 (m, 0.35H), 1.95 (q, J=7.2 Hz, 0.7H), 2.34 (q, J=7.4 Hz, 1.3H), 3.52-3.63 (m, 2H), 4.45 (q, J=7.1 Hz, 0.65H), 4.85 (dt, J=12.6, 7.4 Hz, 0.35H), 5.89 (d, J=6.3 Hz, 0.65H), 6.35 (d, J=12.6 Hz, 0.35H).

¹³C NMR (C₆D₆, 150.9 MHz): δ 14.4 (CH₃), 19.7 (CH₃), 22.78 (CH₃), 22.79 (CH₃), 22.88 (CH₃), 23.1 (CH₂), 24.6 (CH₂), 25.08 (CH₂), 25.09 (CH₂), 28.3 (CH), 28.4 (CH₂), 29.5 (CH₂), 29.78 (CH₂), 29.79 (CH₂), 28.80 (CH₂), 29.95 (CH), 29.99 (CH₂), 30.01 (CH₂), 30.07 (CH₂), 30.09 (CH), 30.1 (CH₂), 30.42 (CH₂), 31.4 (CH₂), 32.3 (CH₂), 36.7 (CH₂), 37.1 (CH₂), 37.62 (CH₂), 37.67 (CH₂), 39.6 (CH₂), 67.2 (CH₂), 70.4 (CH₂), 103.7 (CH), 106.9 (CH), 145.5 (CH), 147.0 (CH).

Compound 12. (2-(dodec-1-en-1-yloxy)ethoxy)benzene: Following the procedure described for compound 9 and starting from dimethyl acetal of dodecanal (8.0 g, 34.7 mmol), 2-phenoxyethanol (12.0 g, 87 mmol) and KHSO₄ (0.15 g, 1.05 mmol), the title compound (1.2 g, 3.8 mmol) was isolated by distillation (bp 125° C., 2.6 Pa) in 11% yield as a colorless liquid (E/Z=40:60).

¹H NMR (CDCl₃, 500 MHz): δ 0.876 and 0.881 (overlapping t, J=7.0 Hz, 3H), 1.18-1.38 (m, 16H), 1.91 (q, J=7.0 Hz, 0.8H), 2.06 (q, J=7.1 Hz, 1.2H), 3.99 (t, J=4.9 Hz, 0.8H), 4.05 (t, J=5.0 Hz, 1.2H), 4.14 (t, J=5.0 Hz, 1.2H), 4.17 (t, J=4.9 Hz, 0.8H), 4.39 (dt, J=6.3, 7.3 Hz, 0.6H), 4.83 (dt, J=12.6, 7.4 Hz, 0.4H), 6.00 (d, J=6.3 Hz, 0.6H), 6.30 (d, J=12.6 Hz, 0.4H), 6.89-6.98 (m, 3H), 7.24-7.31 (m, 2H).

¹³C NMR (CDCl₃, 125.8 MHz): δ 14.1 (CH₃), 22.7 (CH₂), 23.9 (CH₂), 27.7 (CH₂), 29.1 (CH₂), 29.3 (CH₂), 29.36 (CH₂), 29.37 (CH₂), 29.51 (CH₂), 29.53 (CH₂), 29.65 (CH₂), 29.66 (CH₂), 29.67 (CH₂), 29.8 (CH₂), 30.7 (CH₂), 31.9 (CH₂), 66.5 (CH₂), 67.1 (CH₂), 67.3 (CH₂), 70.3 (CH₂), 105.0 (CH), 108.2 (CH), 114.64 (CH), 114.7 (CH), 120.98 (CH), 121.01 (CH), 129.42 (CH), 129.44 (CH), 144.6 (CH), 145.8 (CH), 158.63 (C), 158.69 (C).

Compound 13. 1-(octan-2-yloxy)dodec-1-ene: Following the procedure described for compound 9 and starting from dimethyl acetal of dodecanal (10.0 g, 43.4 mmol), 2-octanol (14.1 g, 108 mmol) and KHSO₄ (0.18 g, 1.30 mmol), the title compound (2.0 g, 6.85 mmol) was isolated by distillation (bp 130° C., 2 Pa) in 16% yield as a colorless liquid (E/Z=30:70).

¹H NMR (CDCl₃, 500 MHz): δ 0.879 and 0.883 (overlapping t, J=7.0 Hz, 6H), 1.17 (d, J=6.2 Hz, 3H), 1.21-1.48 (m, 24.6H), 1.55-1.63 (m, 1.4H), 1.89 (q, J=7.0 Hz, 0.6H), 2.00-2.11 (m, 1.4H), 3.68 (sextet, J=6.1 Hz, 0.7H), 3.73 (sextet, J=6.1 Hz, 0.3H), 4.31 (dt, J=7.3, 6.3 Hz, 0.7H), 4.85 (dt, J=12.4, 7.4 Hz, 0.3H), 5.94 (d, J=6.3 Hz, 0.7H), 6.06 (d, J=12.4 Hz, 0.3H).

¹³C NMR (CDCl₃, 125.8 MHz): δ 14.10 (CH₃), 14.13 (CH₃), 20.0 (CH₃), 20.3 (CH₃), 22.62 (CH₂), 22.64 (CH₂), 22.7 (CH₂), 24.0 (CH₂), 25.4 (CH₂), 25.41 (CH), 27.7 (CH₂), 29.1 (CH₂), 29.3 (CH₂), 29.33 (CH₂), 29.34 (CH₂), 29.37 (CH₂), 29.4 (CH₂), 29.5 (CH₂), 29.6 (CH₂), 29.66 (CH₂), 29.69 (CH₂), 29.72 (CH₂), 29.9 (CH₂), 30.7 (CH₂), 31.83 (CH₂), 31.85 (CH₂), 31.94 (CH₂), 31.95 (CH₂), 36.5 (CH₂), 36.7 (CH₂), 76.4 (CH), 77.7 (CH), 106.2 (CH), 107.0 (CH), 143.8 (CH), 145.0 (CH).

Compound 14. (2-(non-1-en-1-yloxy)ethyl)benzene: Following the procedure described for compound 9 and starting from dimethyl acetal of nonanal (10.0 g, 53.1 mmol), 2-phenylethanol (16.2 g, 132.7 mmol) and KHSO₄ (0.22 g, 1.60 mmol), the title compound (4.0 g, 16.1 mmol) was isolated by distillation (bp 120° C., 2 Pa) in 30% yield as a colorless liquid (E/Z=35:65).

¹H NMR (CD₂Cl₂, 500 MHz): δ 0.876 and 0.885 (overlapping t, J=6.8 Hz, 3H), 1.16-1.37 (m, 10H), 1.89 (q, J=7.0 Hz, 0.7H), 2.02 (q, J=7.0 Hz, 1.3H), 2.89 and 2.91 (overlapping t, J=7.0 Hz, 2H), 3.83 (t, J=7.0 Hz, 0.7H), 3.89 (t, J=7.0 Hz, 1.3H), 4.34 (q, J=6.8 Hz, 0.65H), 4.76 (dt, J=12.6, 7.3 Hz, 0.35H), 5.93 (d, J=6.3 Hz, 0.65H), 6.21 (d, J=12.6 Hz, 0.35H), 7.17-7.24 (m, 3H), 7.26-7.31 (m, 2H).

¹³C NMR (CD₂Cl₂, 125.8 MHz): δ 14.28 (CH₃), 14.30 (CH₃), 23.09 (CH₂), 23.11 (CH₂), 24.4 (CH₂), 28.1 (CH₂), 29.4 (CH₂), 29.57 (CH₂), 29.61 (CH₂), 29.64 (CH₂), 29.67 (CH₂), 30.2 (CH₂), 31.2 (CH₂), 32.28 (CH₂), 32.32 (CH₂), 36.1 (CH₂), 36.7 (CH₂), 70.1 (CH₂), 73.1 (CH₂), 104.9 (CH), 107.6 (CH), 126.64 (CH), 126.66 (CH), 128.69 (CH), 128.73 (CH), 129.3 (CH), 129.4 (CH), 139.0 (C), 139.2 (C), 144.9 (CH), 146.2 (CH).

Compound 15. 1-((3,7-dimethyloct-6-en-1-yl)oxy)non-1-ene: Following the procedure described for compound 9 and starting from dimethyl acetal of nonanal (8.0 g, 42.5 mmol), (±)-citronellol (16.6 g, 106.2 mmol) and KHSO₄ (0.18 g, 1.3 mmol), the title compound (7.0 g, 25.0 mmol) was isolated by distillation (bp 128° C., 2 Pa) in 59% yield as a colorless liquid (E/Z=25:75).

¹H NMR (CDCl₃, 500 MHz): δ 0.88 (t, J=6.9 Hz, 3H), 0.91 (overlapping d, J=6.6 Hz, 3H), 1.12-1.22 (m, 1H), 1.22-1.37 (m, 11H), 1.37-1.47 (m, 1H), 1.53-1.62 (m, 1H), 1.60 (s, 3H), 1.62-1.72 (m, 1H), 1.68 (s, 3H), 1.85-2.08 (m, 4H), 3.61-3.67 (m, 0.5H), 3.67-3.78 (m, 1.5H), 4.32 (dt, J=6.3, 7.3 Hz, 0.75H), 4.76 (dt, J=12.6, 7.3 Hz, 0.25H), 5.09 (t, J=7.1 Hz, 1H), 5.91 (d, J=6.3 Hz, 0.75H), 6.21 (d, J=12.6 Hz, 0.25H).

¹³C NMR (CDCl₃, 125.8 MHz, Z-isomer): δ 14.1 (CH₃), 17.6 (CH₃), 19.6 (CH₃), 22.7 (CH₂), 24.0 (CH₂), 25.5 (CH₂), 25.7 (CH₃), 29.2 (CH₂), 29.3 (CH₂), 29.4 (CH), 29.9 (CH₂), 31.9 (CH₂), 36.6 (CH₂), 37.1 (CH₂), 70.4 (CH₂), 107.1 (CH), 124.7 (CH), 131.2 (C), 144.8 (CH).

Compound 16. (1-methoxy-3-methyl)dodec-1-ene (intermediate used to prepare compounds 25-29): A toluene (500 mL) solution of methoxymethyltriphenylphosphonium chloride (139.7 g, 407.5 mmol) and 2-methylundecanal (50 g, 271.7 mmol) was cooled in an ice bath. Potassium t-butoxide (48.8 g, 434.7 mmol) was added in one portion and the mixture was stirred for 30 min until the exotherm subsided. The mixture was removed from the cold bath and stirred at room temperature until all of the starting aldehyde was consumed (3 h). The mixture was poured into 500 ml of water and stirred for 1 h. After phase separation, the aqueous phase was extracted with EtOAc (3×200 mL). The organic phases were combined, dried over Na₂SO₄, filtered and concentrated to a grainy slurry. The slurry was washed with hexane and filtered. Concentrating the filtrate afforded the crude methyl enol ether. Fractional distillation (bp 80° C., 2 Pa) of this material afforded 50.8 g (239 mmol, 79% yield) of the title compound as a colorless liquid (E/Z=60:40)

¹H NMR (CDCl₃, 600 MHz): δ 0.88 (t, J=6.9 Hz, 3H), 0.94 (d, J=6.9 Hz, 1.2H), 0.97 (d, J=6.8 Hz, 1.8H), 1.14-1.39 (m, 16H), 1.95-2.05 (m, 0.6H), 2.52-2.62 (m, 0.4H), 3.49 (s, 1.8H), 3.55 (s, 1.2H), 4.15 (dd, J=6.3, 9.5 Hz, 0.4H), 4.58 (dd, J=8.6, 12.7 Hz, 0.6H), 5.82 (d, J=6.3 Hz, 0.4H), 6.25 (d, J=12.7 Hz, 0.6H).

¹³C NMR (CDCl₃, 150.9 MHz): δ 14.1 (CH₃), 21.4 (CH₃), 22.2 (CH₃), 22.7 (CH₂), 27.4 (CH₂), 27.5 (CH₂), 28.9 (CH), 29.38 (CH₂), 29.39 (CH₂), 29.68 (CH₂), 29.69 (CH₂), 29.72 (CH₂), 29.74 (CH₂), 29.78 (CH₂), 29.82 (CH₂), 31.95 (CH₂), 31.96 (CH₂), 32.8 (CH), 37.7 (CH₂), 38.1 (CH₂), 55.9 (CH₃), 59.4 (CH₃), 109.7 (CH), 113.9 (CH), 145.0 (CH), 145.9 (CH).

Compound 17. 1-methoxydodeca-1,11-diene (intermediate used to prepare compound 30): Following the procedure described for compound 14 and starting from 10-undecenal, the title compound was isolated by fractional distillation (bp of the crude reaction mixture (bp 80° C., 100 mtorr) in 45% yield as a colorless liquid (E/Z=50:50).

¹H NMR (CDCl₃, 500 MHz): δ 1.21-1.43 (m, 12H), 1.91 (q, J=7.0 Hz, 1H), 2.00-2.08 (m, 3H), 3.49 (s, 1.5H), 3.57 (s, 1.5H), 4.33 (q, J=7.3 Hz, 0.5H), 4.72 (dt, J=7.4, 12.6 Hz, 0.5H), 4.92 (d, J=10.2 Hz, 1H), 4.99 (d, J=17.1 Hz, 1H), 5.81 (ddt, J=6.8, 10.2, 17.1 Hz, 1H), 5.86 (d, J=6.2 Hz, 0.5H), 6.27 (d, J=12.6 Hz, 0.5H).

¹³C NMR (CDCl₃, 125.8 MHz): δ 23.9 (CH₂), 27.7 (CH₂), 28.96 (CH₂), 29.02 (CH₂), 29.2 (CH₂), 29.3 (CH₂), 29.46 (CH₂), 29.49 (CH₂), 29.8 (CH₂), 30.8 (CH₂), 33.8 (CH₂), 55.9 (CH₃), 59.5 (CH₃), 103.2 (CH), 107.2 (CH), 114.08 (CH₂), 114.11 (CH₂), 139.2 (CH), 139.3 (CH), 145.9 (CH), 146.9 (CH).

Compound 18. (5E)-1-methoxyundeca-1,5-diene (intermediate used to prepare compound 31): Following the procedure described for compound 14 and starting from trans-4-decenal, the title compound was isolated by fractional distillation (bp 80° C., 26.7 Pa) in 25% yield as a colorless liquid (E/Z=60:40).

¹H NMR (CDCl₃, 500 MHz): δ 0.88 (t, J=6.9 Hz, 3H), 1.21-1.38 (m, 6H), 1.93-2.05 (m, 5H), 2.05-2.15 (m, 1H), 3.49 (s, 1.8H), 3.57 (s, 1.2H), 4.34 (q, J=6.9 Hz, 0.6H), 4.73 (dt, J=7.1, 12.6 Hz, 0.4H), 5.33-5.46 (m, 2H), 5.86 (d, J=6.3 Hz, 0.4H), 6.29 (d, J=12.6 Hz, 0.6H).

¹³C NMR (CDCl₃, 125.8 MHz): δ 14.1 (CH₃), 22.6 (CH₂), 24.0 (CH₂), 28.0 (CH₂), 29.3 (CH₂), 31.4 (CH₂), 32.6 (CH₂), 32.7 (CH₂), 33.9 (CH₂), 55.9 (CH₃), 59.5 (CH₃), 102.6 (CH), 106.4 (CH), 129.58 (CH₂), 129.7 (CH₂), 130.7 (CH), 131.0 (CH), 146.1 (CH), 147.1 (CH).

Compound 19. (5-methoxypent-4-en-2-yl)benzene (intermediate used to prepare compound 33): Following the procedure described for compound 14 and starting from 3-phenylbutanal, the title compound was isolated by fractional distillation (bp 70° C., 8 Pa) in 60% yield as a colorless liquid (E/Z=50:50).

¹H NMR (CDCl₃, 600 MHz): δ 1.24 and 1.25 (overlapping d, J=6.8 Hz, 3H), 2.09-2.16 (m, 0.5H), 2.19-2.26 (m, 0.5H), 2.29-2.41 (m, 1H), 2.65-2.77 (m, 1H), 3.44 (s, 1.5H), 3.52 (s, 1.5H), 4.25 (q, J=7.0 Hz, 0.5H), 4.62 (dt, J=7.6, 12.7 Hz, 0.5H), 5.84 (d, J=6.3 Hz, 0.5H), 6.24 (d, J=12.7 Hz, 0.5H), 7.14-7.19 (m, 2H), 7.19-7.23 (m, 1H), 7.25-7.31 (m, 2H).

¹³C NMR (CDCl₃, 150.9 MHz): δ 21.1 (CH₃), 21.8 (CH₃), 32.3 (CH₂), 36.6 (CH₂), 40.1 (CH), 41.0 (CH), 55.9 (CH₃), 59.4 (CH₃), 101.1 (CH), 105.1 (CH), 128.8 (CH), 125.9 (CH), 127.0 (CH), 127.1 (CH), 128.2 (CH), 128.3 (CH), 146.7 (CH), 147.1 (C), 147.4 (C), 147.9 (CH).

Compound 20. (4R)-4-(5-methoxypent-4-en-2-yl)-1-methylcyclohex-1-ene (intermediate used to prepare compound 34): Following the procedure described for compound 14 and starting from 3-((R)-4-methylcyclohex-3-en-1-yl)butanal, the title compound was isolated by fractional distillation (bp 94° C., 20 Pa) in 48% yield as a colorless liquid (mixture of diastereomers, E/Z=40:60).

¹H NMR (CDCl₃, 500 MHz): δ 0.82-0.87 (overlapping d, 3H), 1.15-1.46 (m, 3H), 1.64 (s, 3H), 1.66-1.83 (m, 2.4H), 1.88-2.05 (m, 4H), 2.08-2.16 (m, 0.6H), 3.51 (s, 1.2H), 3.57 (s, 1.8H), 4.33 (overlapping q, J=7.0 Hz, 0.6H), 4.70 (overlapping dt, J=7.6, 12.6 Hz, 0.4H), 5.35-5.40 (m, 1H), 5.90 (d, J=6.3 Hz, 0.6H), 6.25 (d, J=12.6 Hz, 0.4H).

¹³C NMR (CDCl₃, 125.8 MHz): δ 15.5 (CH₃), 15.8 (CH₃), 16.0 (CH₃), 16.2 (CH₃), 23.48 (CH₃), 23.49 (CH₃), 25.40 (CH₂), 25.44 (CH₂), 27.26 (CH₂), 27.28 (CH₂), 27.55 (CH₂), 27.64 (CH₂), 28.3 (CH₂), 28.5 (CH₂), 29.74 (CH₂), 29.76 (CH₂), 30.8 (CH₂), 30.88 (CH₂), 30.90 (CH₂), 30.96 (CH₂), 32.1 (CH₂), 32.4 (CH₂), 37.53 (CH), 37.58 (CH), 37.7 (CH), 37.8 (CH), 38.0 (CH), 38.1 (CH), 38.4 (CH), 55.91 (CH₃), 55.92 (CH₃), 59.4 (CH₃), 101.6 (CH), 101.7 (CH), 105.78 (CH), 105.83 (CH), 121.00 (CH), 121.04 (CH), 121.11 (CH), 121.17 (CH), 133.92 (C), 133.94 (C), 133.98 (C), 134.0 (C), 146.52 (CH), 146.56 (CH), 147.49 (CH), 146.52 (CH).

Compound 21. 1-methoxy-4,8-dimethylnona-1,7-diene (intermediate used to prepare compound 32): Following the procedure described for compound 14 and starting from (±)-citronellal, the title compound was isolated by fractional distillation (bp 70° C., 26.7 Pa) in 13% yield as a colorless liquid (E/Z=54:46).

¹H NMR (CDCl₃, 500 MHz): δ 0.86 and 0.88 (both d, J=6.7 Hz, 3H), 1.07-1.19 (m, 1H), 1.30-1.39 (m, 1H), 1.39-1.50 (m, 1H), 1.60 (s, 3H), 1.68 (s, 3H), 1.72-1.79 (m, 0.54H), 1.88-2.09 (m, 3.46H), 3.51 (s, 1.62H), 3.57 (s, 1.38H), 4.33 (dt, J=6.3, 7.3 Hz, 0.46H), 4.70 (dt, J=7.6, 12.6 Hz, 0.541H), 5.10 (t, J=7.2 Hz, 1H), 5.91 (d, J=6.3 Hz, 0.46H), 6.25 (d, J=12.6 Hz, 0.54H).

¹³C NMR (CDCl₃, 125.8 MHz): δ 17.62 (CH₃), 17.64 (CH₃), 19.2 (CH₃), 19.4 (CH₃), 25.64 (CH₂), 25.66 (CH₂), 25.7 (CH₃), 30.9 (CH₂), 32.9 (CH), 33.4 (CH), 34.9 (CH₂), 36.4 (CH₂), 36.6 (CH₂), 55.9 (CH₃), 59.4 (CH₃), 101.3 (CH), 105.4 (CH), 124.9 (CH), 125.1 (CH), 130.9 (C), 131.1 (C), 146.6 (CH), 147.6 (CH).

Compound 22. 1-(tert-butyl)-4-(4-methoxy-2-methylbut-3-en-1-yl)benzene (intermediate used to prepare compound 35): Following the procedure described for compound 14 and starting from 3-(4-(tert-butyl)phenyl)-2-methylpropanal, the title compound was isolated by fractional distillation (bp 100° C., 2 Pa) in 81% yield as a colorless liquid (E/Z=60:40).

¹H NMR (CDCl₃, 500 MHz): δ 0.95 (d, J=6.7 Hz, 1.2H), 0.98 (d, J=6.7 Hz, 1.8H), 1.30 and 1.31 (both s, 9H), 2.35 (septet, J=7.1 Hz, 0.6H), 2.49 (dd, J=7.6, 13.4 Hz, 1H), 2.60 and 2.61 (overlapping dd, J=6.7, 13.4 Hz, 1H), 2.87-2.97 (m, 0.4H), 3.46 (s, 1.2H), 3.47 (s, 1.8H), 4.24 (dd, J=6.3, 9.2 Hz, 0.4H), 4.69 (dd, J=8.0, 12.7 Hz, 0.6H), 5.78 (d, J=6.3 Hz, 0.4H), 6.22 (d, J=12.7 Hz, 0.6H), 7.04-7.09 (m, 1.2H), 7.09-7.13 (m, 0.8H), 7.25-7.31 (m, 2H).

¹³C NMR (CDCl₃, 125.8 MHz): δ 20.7 (CH₃), 21.1 (CH₃), 30.6 (CH), 31.4 (CH₃), 34.31 (C), 34.33 (C), 34.6 (CH), 43.2 (CH₂), 44.3 (CH₂), 55.9 (CH₃), 59.4 (CH₃), 109.0 (CH), 113.0 (CH), 124.8 (CH), 124.9 (CH), 128.86 (CH), 128.91 (CH), 137.7 (C), 137.9 (C), 145.1 (CH), 146.2 (CH), 148.3 (C), 148.4 (C).

Compound 23. 4-(2-methoxyvinyl)-1,3-dimethylcyclohex-1-ene (intermediate used to prepare compound of formula (I)): Following the procedure described for compound 14 and starting from 2,4-dimethylcyclohex-3-ene-1-carbaldehyde, the title compound was isolated by fractional distillation (bp 65° C., 67 Pa) in 68% yield as a colorless liquid (mixture of diastereomers, E/Z=42:58).

¹H NMR (C₆D₆, 500 MHz): 0.93 (d, J=7.0 Hz, 0.3H), 1.02 (d, J=7.0 Hz, 1.8H) 1.04 (d, J=7.2 Hz, 0.2H), 1.34 (d, J=7.0 Hz, 0.7H), 1.42-1.60 (m, 1.7H), 1.62 and 1.63 (both s, 3H), 1.67-1.74 (m, 0.7), 1.75-2.04 (m, 3H), 2.14-2.26, 2.38-2.48 and 2.51-2.61 (all m, 0.6H), 3.12 (s, 0.7H), 3.14 (s, 0.2H), 3.18 (s, 2.1H), 4.26 (dd, J=6.3, 9.5 Hz, 0.23H), 4.43 (dd, J=6.3, 9.5 Hz, 0.07H), 4.57 (dd, J=9.1, 12.7 Hz, 0.6H), 4.75 (dd, J=9.1, 12.7 Hz, 0.1H), 5.27-5.31 (m, 0.7H), 5.31-5.35 (m, 0.3H), 5.73 (d, J=6.3 Hz, 0.3H), 6.35 and 6.36 (overlapping d, J=12.7 Hz, 0.7H)

¹³C NMR (C₆D₆, 125.8 MHz): δ 17.7 (CH₃), 17.9 (CH₃), 20.7 (CH₃), 20.9 (CH₃), 23.71 (CH₃), 23.74 (CH₃), 23.76 (CH₃), 23.78 (CH₃), 27.1 (CH₂), 27.6 (CH₂), 28.6 (CH₂), 28.9 (CH₂), 30.0 (CH₂), 30.2 (CH₂), 30.3 (CH₂), 31.3 (CH₂), 33.2 (CH), 33.6 (CH), 34.6 (CH), 37.1 (CH), 37.14 (CH), 37.2 (CH), 37.6 (CH), 41.5 (CH), 55.3 (CH₃), 59.0 (CH₃), 104.2 (CH), 107.4 (CH), 108.1 (CH), 112.0 (CH), 127.7 (CH), 127.75 (CH), 127.79 (CH), 127.84 (CH), 132.6 (C), 132.7 (C), 132.9 (C), 133.0 (C), 146.48 (CH), 146.50 (CH), 147.67 (CH), 147.72 (CH).

Compound 24. 1-(4-methoxybut-3-en-1-yl)-4,4-dimethylcyclohex-1-ene (intermediate used to prepare compound of formula (I)): Following the procedure described for compound 14 and starting from 3-(4,4-dimethylcyclohex-1-en-1-yl)propanal, the title compound was isolated by fractional distillation (bp 80° C., 26.7 Pa) in 23% yield as a colorless liquid (E/Z=50:50).

¹H NMR (CDCl₃, 500 MHz): δ 0.881 and 88.3 (both s, 6H), 1.34 and 1.35 (overlapping t, J=6.5 Hz, 2H), 1.75-1.78 (m, 2H), 1.89-1.96 (m, 2H), 1.96-2.08 (m, 3H), 2.15-2.21 (m, 1H), 3.49 (s, 1.5H), 3.57 (s, 1.5H), 4.32 (q, J=7.1 Hz, 0.5H), 4.71 (dt, J=6.9, 12.6 Hz, 0.5H), 5.29-5.34 (m, 1H), 5.85 (d, J=6.3 Hz, 0.5H), 6.28 (d, J=12.6 Hz, 0.5H).

¹³C NMR (CDCl₃, 125.8 MHz): δ 22.2 (CH₂), 26.0 (CH₂), 26.1 (CH₂), 26.2 (CH₂), 28.19 (CH₃), 28.21 (CH₃), 28.49 (C), 28.52 (C), 35.7 (CH₂), 35.8 (CH₂), 37.6 (CH₂), 38.8 (CH₂), 39.31 (CH₂), 39.32 (CH₂), 55.9 (CH₃), 59.5 (CH₃), 102.9 (CH), 106.7 (CH), 120.0 (CH), 120.3 (CH), 135.7 (C), 136.0 (C), 145.9 (CH), 146.9 (CH).

Compound 25. (2-((3-methyldodec-1-en-1-yl)oxy)ethyl)benzene: Compound 16 (8.0 g, 37.7 mmol), 2-phenylethanol (11.5 g, 94.2 mmol) and KHSO₄ (0.153 g, 1.12 mmol) were added to a 35 ml, round-bottomed flask equipped with a distillation head and vacuum pump. The mixture was heated at 150° C. (oil bath) under vacuum (450 mbar) for 45 min and another 45 min while reducing the vacuum to 20 mbar. The mixture was allowed to cool then placed under vacuum (typically 67 Pa) and heated (120° C. oil bath) while allowing excess phenylethanol to distill from the reaction flask. The vacuum was progressively lowered to 2 Pa, and the bath temperature increased to 200° C. while allowing the reaction product to distill from the flask. Fractions rich in the enol ether were combined and subjected to short-path distillation (bp 140° C., 1.3 Pa) to afford 5.81 g (19.2 mmol, 51% yield) of the title compound as a colorless oil (E/Z=43:57).

¹H NMR (CDCl₃, 600 MHz): δ 0.88 (t, J=6.8 Hz, 3H), 0.93 (d, J=6.8 Hz, 1.7H), 0.95 (d, J=6.8 Hz, 1.3H), 1.12-1.33 (m, 16H), 1.94-2.03 (m, 0.43H), 2.53-2.63 (m, 0.57H), 2.91 (t, J=7.2 Hz, 1.14H), 2.95 (t, J=7.2 Hz, 0.86H), 3.84 (t, J=7.2 Hz, 0.86H), 3.86-3.93 (m, 1.14H), 4.16 (dd, J=6.3, 9.4 Hz, 0.57H), 4.62 (dd, J=8.7, 12.7 Hz, 0.43H), 5.88 (d, J=6.3 Hz, 0.57H), 6.20 (d, J=12.7 Hz, 0.43H), 7.19-7.25 (m, 3H), 7.26-7.31 (m, 2H).

¹³C NMR (CDCl₃, 150.9 MHz): δ 14.1 (CH₃), 21.4 (CH₃), 22.1 (CH₃), 22.7 (CH₂), 22.71 (CH₂), 27.4 (CH₂), 27.5 (CH₂), 29.0 (CH), 29.36 (CH₂), 29.40 (CH₂), 29.66 (CH₂), 29.70 (CH₂), 29.75 (CH₂), 29.83 (CH₂), 31.9 (CH₂), 32.0 (CH₂), 32.9 (CH), 35.9 (CH₂), 36.4 (CH₂), 37.7 (CH₂), 38.0 (CH₂), 69.7 (CH₂), 72.7 (CH₂), 111.0 (CH), 114.2 (CH), 126.3 (CH), 126.4 (CH), 128.4 (CH), 128.9 (CH), 129.0 (CH), 138.37 (C), 138.41 (C), 143.5 (CH), 144.8 (CH).

Compound 26. (2-((3-methyldodec-1-en-1-yl)oxy)ethoxy)benzene: Following the procedure described for compound 25 and starting from compound 16 (8 g, 37.7 mmol) and 2-phenoxyethanol (13.0 g, 94.2 mmol), the title compound was isolated by distillation (bp 140° C., 2.7 Pa) in 47% yield as a colorless liquid (E/Z=64:36).

¹H NMR (CDCl₃, 500 MHz): δ 0.88 (overlapping t, J=7.0 Hz, 3H), 0.92 (d, J=6.8 Hz, 1.1H), 0.98 (d, J=6.8 Hz, 1.9H), 1.12-1.34 (m, 16H), 1.94-2.08 (m, 0.64H), 2.55-2.67 (m, 0.36H), 3.97-4.01 (m, 1.28H), 4.01-4.08 (m, 0.72H), 4.13 (t, J=5.0 Hz, 0.72H), 4.18 (t, J=5.0 Hz, 1.28H), 4.21 (dd, J=6.2, 9.5 Hz, 0.36H), 4.69 (dd, J=8.7, 12.6 Hz, 0.64H), 5.97 (d, J=6.2 Hz, 0.36H), 6.28 (d, J=12.6 Hz, 0.64H), 6.89-6.98 (m, 3H), 7.24-7.31 (m, 2H).

¹³C NMR (CDCl₃, 125.8 MHz): δ 14.1 (CH₃), 21.3 (CH₃), 22.0 (CH₃), 22.7 (CH₂), 27.38 (CH₂), 27.43 (CH₂), 28.9 (CH), 29.37 (CH₂), 29.38 (CH₂), 29.67 (CH₂), 29.69 (CH₂), 29.71 (CH₂), 29.76 (CH₂), 29.8 (CH₂), 31.93 (CH₂), 31.94 (CH₂), 32.8 (CH), 66.5 (CH₂), 67.0 (CH₂), 67.3 (CH₂), 70.2 (CH₂), 111.3 (CH), 114.6 (CH), 114.7 (CH), 114.8 (CH), 120.97 (CH), 121.0 (CH), 129.43 (CH), 129.44 (CH), 143.7 (CH), 144.8 (CH), 158.64 (C), 158.7 (C).

Compound 27. 1-(((Z)-hex-3-en-1-yl)oxy)-3-methyldodec-1-ene: Following the procedure described for compound 25 and starting from compound 16 (8 g, 37.7 mmol) and cis-3-hexen-1-ol (9.43 g, 94.2 mmol), the title compound (8.2 g, 29.1 mmol) was isolated by distillation (bp 125° C., 2 Pa) in 77% yield as a colorless liquid (E/Z=40:60).

¹H NMR (CDCl₃, 600 MHz): δ 0.88 (t, J=7.0 Hz, 3H), 0.93 (d, J=6.8 Hz, 1.8H), 0.94-0.99 (m, 4.2H), 1.12-1.34 (m, 16H), 1.94-2.03 (m, 0.4H), 2.06 (pentet, J=7.5 Hz, 2H), 2.35 and 2.38 (overlapping q, J=7.1 Hz, 2H), 2.54-2.63 (m, 0.6H), 3.62 (t, J=7.0 Hz, 0.8H), 3.64-3.71 (m, 1.2H), 4.14 (dd, J=6.3, 9.4 Hz, 0.6H), 4.63 (dd, J=8.6, 12.6 Hz, 0.4H), 5.30-5.38 (m, 1H), 5.44-5.53 (m, 1H), 5.88 (d, J=6.3 Hz, 0.6H), 6.19 (d, J=12.6 Hz, 0.4H).

¹³C NMR (CDCl₃, 150.9 MHz): δ 14.1 (CH₃), 14.3 (CH₃), 20.6 (CH₂), 20.7 (CH₂), 21.4 (CH₃), 22.1 (CH₃), 22.7 (CH₂), 27.39 (CH₂), 27.42 (CH₂), 27.5 (CH₂), 28.0 (CH₂), 28.9 (CH), 29.38 (CH₂), 29.40 (CH₂), 29.67 (CH₂), 29.71 (CH₂), 29.76 (CH₂), 29.77 (CH₂), 29.8 (CH₂), 31.9 (CH₂), 32.0 (CH₂), 32.9 (CH), 37.7 (CH₂), 38.1 (CH₂), 68.6 (CH₂), 71.6 (CH₂), 110.9 (CH), 113.9 (CH), 124.2 (CH), 124.3 (CH), 134.0 (CH), 134.1 (CH), 143.7 (CH), 144.9 (CH).

Compound 28. 3-methyl-1-(((Z)-oct-3-en-1-yl)oxy)dodec-1-ene: Following the procedure described for compound 25 and starting from compound 16 (8 g, 37.7 mmol) and cis-3-octen-1-ol (12.1 g, 94.2 mmol), the title compound (6.0 g, 19.4 mmol) was isolated by distillation (bp 135° C., 2 Pa) in 52% yield as a colorless liquid (E/Z=40:60).

¹H NMR (CDCl₃, 500 MHz): δ 0.88-0.92 (overlapping t, 6H), 0.93 (d, J=6.7 Hz, 1.8H), 0.96 (d, J=6.8 Hz, 1.2H), 1.14-1.30 (m, 16H), 1.30-1.37 (m, 4H), 1.94-2.02 (m, 0.4H), 2.02-2.09 (m, 2H), 2.35 and 2.38 (overlapping q, J=7.2 Hz, 2H), 2.54-2.64 (m, 0.6H), 3.62 (t, J=7.1 Hz, 0.8H), 3.64-3.72 (m, 1.2H), 4.14 (dd, J=6.3, 9.3 Hz, 0.6H), 4.63 (dd, J=8.7, 12.7 Hz, 0.4H), 5.32-5.41 (m, 1H), 5.44-5.54 (m, 1H), 5.88 (d, J=6.3 Hz, 0.6H), 6.19 (d, J=12.6 Hz, 0.4H).

¹³C NMR (CDCl₃, 125.8 MHz): δ 14.0 (CH₃), 14.1 (CH₃), 21.4 (CH₃), 22.1 (CH₃), 22.3 (CH₂), 22.7 (CH₂), 27.05 (CH₂), 27.06 (CH₂), 27.38 (CH₂), 27.46 (CH₂), 27.51 (CH₂), 28.1 (CH₂), 28.9 (CH), 29.37 (CH₂), 29.4 (CH₂), 29.67 (CH₂), 29.7 (CH₂), 29.75 (CH₂), 29.76 (CH₂), 29.8 (CH₂), 31.81 (CH₂), 31.82 (CH₂), 31.93 (CH₂), 31.95 (CH₂), 32.9 (CH), 37.7 (CH₂), 38.1 (CH₂), 68.7 (CH₂), 71.6 (CH₂), 110.9 (CH), 113.9 (CH), 124.7 (CH), 124.8 (CH), 132.4 (CH), 132.5 (CH), 143.7 (CH), 144.9 (CH).

Compound 29. 3-methyl-1-(octan-3-yloxy)dodec-1-ene: Following the procedure described for compound 25 and starting from compound 16 (8.0 g, 37.7 mmol) and 3-octanol (12.3 g, 94.2 mmol), the title compound (5.8 g, 18.7 mmol) was isolated by distillation (bp 130° C., 2 Pa) in 50% yield as a colorless liquid (mixture of diastereomers, E/Z=35:65).

¹H NMR (CDCl₃, 500 MHz): δ 0.86-0.92 (overlapping t, 9H), 0.93 (d, J=6.8 Hz, 1.9H), 0.96 (d, J=6.7 Hz, 1.1H), 1.14-1.38 (m, 26H), 1.91-2.02 (m, 0.35H), 2.53-2.66 (m, 0.65), 3.45 (pentet, J=6.0 Hz, 0.65H), 3.48-3.54 (m, 0.35H), 4.06 (overlapping dd, J=6.3, 9.2 Hz, 0.65H), 4.72 (dd, J=8.6, 12.3 Hz, 0.35H), 5.90 (d, J=6.3 Hz, 0.65H), 6.02 (d, J=12.3 Hz, 0.35H).

¹³C NMR (CDCl₃, 125.8 MHz): δ 9.54 (CH₃), 9.57 (CH₃), 9.60 (CH₃), 9.63 (CH₃), 14.05 (CH₃), 14.06 (CH₃), 14.13 (CH₃), 21.52 (CH₃), 21.54 (CH₃), 22.02 (CH₃), 22.03 (CH₃), 22.64 (CH₂), 22.67 (CH₂), 22.72 (CH₂), 22.73 (CH₂), 24.97 (CH₂), 24.99 (CH₂), 25.03 (CH₂), 25.04 (CH₂), 26.93 (CH₂), 26.95 (CH₂), 26.97 (CH₂), 27.31 (CH₂), 27.33 (CH₂), 27.40 (CH₂), 27.43 (CH₂), 27.51 (CH₂), 27.55 (CH₂), 28.93 (CH), 28.94 (CH), 33.66 (CH₂), 33.74 (CH₂), 34.0 (CH₂), 34.06 (CH₂), 37.81 (CH₂), 37.85 (CH₂), 38.0 (CH₂), 81.7 (CH), 81.8 (CH), 83.07 (CH), 83.08 (CH), 112.37 (CH), 112.38 (CH), 112.53 (CH), 112.54 (CH), 143.6 (CH), 143.65 (CH), 144.57 (CH), 144.62 (CH).

Compound 30. (2-(dodeca-1,11-dien-1-yloxy)ethyl)benzene: Following the procedure described for compound 25 and starting from compound 17 (8 g, 40.7 mmol) and 2-phenylethanol (12.4 g, 102 mmol), the title compound (3.6 g, 12.4 mmol) was isolated by distillation (bp 140° C., 2 Pa) in 31% yield as a colorless liquid (E/Z=30:70).

¹H NMR (CDCl₃, 500 MHz): δ 1.23-1.34 (m, 10H), 1.34-1.42 (m, 2H), 1.89 (q, J=6.9 Hz, 0.6H), 1.99-2.09 (m, 3.4H), 2.92 (t, J=7.2 Hz, 1.4H), 2.94 (t, J=7.2 Hz, 0.6H), 3.84 (t, J=7.2 Hz, 0.6H), 3.91 (t, J=7.2 Hz, 1.4H), 4.35 (q, J=7.2 Hz, 0.7H), 4.77 (dt, J=7.4, 12.6 Hz, 0.3H), 4.92 (d, J=10.2 Hz, 1H), 5.00 (d, J=17.2 Hz, 1H), 5.76-5.86 (m, 1H), 5.92 (d, J=6.1 Hz, 0.7H), 6.22 (d, J=12.6 Hz, 0.3H), 7.19-7.25 (m, 3H), 7.26-7.32 (m, 2H).

¹³C NMR (CDCl₃, 125.8 MHz): δ 24.0 (CH₂), 27.7 (CH₂), 28.94 (CH₂), 28.96 (CH₂), 29.0 (CH₂), 29.14 (CH₂), 29.17 (CH₂), 29.28 (CH₂), 29.43 (CH₂), 29.46 (CH₂), 29.50 (CH₂), 29.8 (CH₂), 30.7 (CH₂), 33.82 (CH₂), 33.84 (CH₂), 35.8 (CH₂), 36.4 (CH₂), 69.7 (CH₂), 72.7 (CH₂), 104.6 (CH), 107.6 (CH), 114.09 (CH₂), 114.1 (CH₂), 126.33 (CH), 126.36 (CH), 128.38 (CH), 128.42 (CH), 128.9 (CH), 129.0 (CH), 138.3 (CH), 138.4 (CH), 139.2 (CH), 139.3 (CH), 144.5 (CH), 145.8. (CH).

Compound 31. (2-(((5E)-undeca-1,5-dien-1-yl)oxy)ethyl)benzene: Following the procedure described for compound 25 and starting from compound 18 (8 g, 43.9 mmol) and 2-phenylethanol (13.4 g, 110 mmol), the title compound (3.97 g, 14.6 mmol) was isolated by distillation (bp 135° C., 2 Pa) in 33% yield as a colorless liquid (E/Z=40:60).

¹H NMR (CDCl₃, 500 MHz): δ 0.875 and 0.88 (overlapping t, J=6.9 Hz, 3H), 1.21-1.38 (m, 6H), 1.92-2.09 (m, 4.8H), 2.12 (q, J=7.3 Hz, 1.2H), 2.92 and 2.94 (overlapping t, J=7.2 Hz, 2H), 3.84 (t, J=7.2 Hz, 0.8H), 3.91 (t, J=7.2 Hz, 1.2H), 4.36 (q, J=6.9 Hz, 0.6H), 4.77 (dt, J=7.2, 12.6 Hz, 0.4H), 5.32-5.46 (m, 2H), 5.93 (d, J=6.3 Hz, 0.6H), 6.23 (d, J=12.6 Hz, 0.4H), 7.18-7.24 (m, 3H), 7.26-7.32 (m, 2H).

¹³C NMR (CDCl₃, 125.8 MHz): δ 14.08 (CH₃), 14.1 (CH₃), 22.55 (CH₂), 22.57 (CH₂), 24.1 (CH₂), 28.1 (CH₂), 29.30 (CH₂), 29.33 (CH₂), 31.38 (CH₂), 31.41 (CH₂), 32.57 (CH₂), 32.59 (CH₂), 32.7 (CH₂), 33.8 (CH₂), 35.8 (CH₂), 36.4 (CH₂), 69.7 (CH₂), 72.8 (CH₂), 104.0 (CH), 106.8 (CH), 126.3 (CH), 126.4 (CH), 128.38 (CH), 128.43 (CH), 128.9 (CH), 129.0 (CH), 129.5 (CH), 129.8 (CH), 130.7 (CH), 131.0 (CH), 138.3 (C), 138.4 (C), 144.6 (CH), 146.0 (CH).

Compound 32. (2-((4,8-dimethylnona-1,7-dien-1-yl)oxy)ethyl)benzene: Following the procedure described for compound 25 and starting from compound 21 (8 g, 43.9 mmol) and 2-phenylethanol (13.4 g, 109.7 mmol), the title compound (1.81 g, 6.64 mmol) was isolated by distillation (bp 135° C., 2 Pa) in 15% yield as a colorless liquid (E/Z=35:65).

¹H NMR (CDCl₃, 500 MHz): δ 0.85 and 0.87 (both d, J=6.6 Hz, 3H), 1.06-1.19 (m, 1H), 1.28-1.49 (m, 2H), 1.60 and 1.61 (both s, 3H), 1.68 (s, 3H), 1.69-1.78 (m, 0.35H), 1.86-2.10 (m, 3.65H), 2.92 and 2.95 (overlapping t, J=7.2 Hz, 2H), 3.86 (t, J=7.2 Hz, 0.7H), 3.91 (t, J=7.2 Hz, 1.3H), 4.35 (dt, J=6.4, 7.4 Hz, 0.65H), 4.76 (dt, J=7.6, 12.6 Hz, 0.35H), 5.07-5.13 (m, 1H), 5.97 (d, J=6.4 Hz, 0.65H), 6.20 (d, J=12.6 Hz, 0.35H), 7.19-7.25 (m, 2H), 7.26-7.32 (m, 3H).

¹³C NMR (CDCl₃, 125.8 MHz): δ 17.6 (CH₃), 19.2 (CH₃), 19.4 (CH₃), 25.6 (CH₂), 25.7 (CH₂), 25.73 (CH₃), 25.74 (CH₃), 31.0 (CH₂), 33.0 (CH), 33.4 (CH), 35.0 (CH₂), 35.8 (CH₂), 36.4 (CH₂), 36.41 (CH₂), 36.7 (CH₂), 69.8 (CH₂), 72.8 (CH₂), 102.7 (CH), 105.8 (CH), 124.9 (CH), 125.1 (CH), 126.3 (CH), 126.4 (CH), 128.4 (CH), 128.44 (CH), 128.9 (CH), 129.0 (CH), 130.9 (C), 131.1 (C), 138.3 (C), 138.4 (C), 145.1 (CH), 146.5 (CH).

Compound 33. (5-phenethoxypent-4-en-2-yl)benzene: Following the procedure described for compound 25 and starting from compound 19 (8 g, 45.4 mmol) and 2-phenylethanol (13.9 g, 113.5 mmol), the title compound (5.2 g, 19.5 mmol) was isolated by distillation (bp 138° C., 2 Pa) in 43% yield as a colorless liquid (E/Z=35:65).

¹H NMR (CDCl₃, 600 MHz): δ 1.23 (d, J=7.0 Hz, 3H), 2.08-2.14 (m, 0.35H), 2.18-2.24 (m, 0.35H), 2.30-2.39 (m, 1.3H), 2.64-2.76 (m, 1H), 2.89 and 2.90 (overlapping t, J=7.1 Hz, 2H), 3.80 (t, J=7.1 Hz, 0.7H), 3.83-3.91 (m, 1.3H), 4.27 (q, J=7.0 Hz, 0.65H), 4.68 (dt, J=7.6, 12.6 Hz, 0.35H), 5.92 (d, J=6.3 Hz, 0.65H), 6.18 (d, J=12.6 Hz, 0.35H), 7.15-7.24 (m, 6H), 7.25-7.32 (m, 4H).

¹³C NMR (CDCl₃, 150.9 MHz): δ 21.1 (CH₃), 21.7 (CH₃), 32.5 (CH₂), 35.7 (CH₂), 36.4 (CH₂), 36.7 (CH₂), 40.1 (CH), 40.9 (CH), 69.7 (CH₂), 72.7 (CH₂), 102.5 (CH), 105.5 (CH), 125.8 (CH), 125.9 (CH), 126.35 (CH), 126.37 (CH), 127.0 (CH), 127.1 (CH), 128.1 (CH), 128.3 (CH), 128.39 (CH), 128.42 (CH), 128.9 (CH), 129.0 (CH), 138.3 (C), 145.2 (CH), 146.8 (CH), 147.1 (C), 147.4 (C).

### Compound 34. (2-((4-((R)-4-methylcyclohex-3-en-1-yl)pent-1-en-1-yl)oxy)ethyl)benzene

Following the procedure described for compound 25 and starting from compound 20 (8 g, 41.2 mmol) and 2-phenylethanol (12.6 g, 103 mmol), the title compound (3.67 g, 12.5 mmol) was isolated by distillation (bp 135° C., 2 Pa) in 31% yield as a colorless liquid (mixture of diastereomers, E/Z=40:60).

¹H NMR (CDCl₃, 500 MHz): δ 0.80-0.86 (overlapping d, J=6.7 Hz, 3H), 1.15-1.46 (m, 3H), 1.63 (s, 3H), 1.65-1.82 (m, 2.4H), 1.87-2.04 (m, 4H), 2.08-2.17 (m, 0.6H), 2.91 (t, J=7.1 Hz, 1.2H), 2.94 (t, J=7.2 Hz, 0.8H), 3.85 (t, J=7.2 Hz, 0.8H), 3.90 (t, J=7.1 Hz, 1.2H), 4.35 (q, J=7.0 Hz, 0.6H), 4.74 (overlapping dt, J=7.6, 12.6 Hz, 0.4H), 5.35-5.39 (m, 1H), 5.96 (d, J=6.3 Hz, 0.6H), 6.20 (d, J=12.6 Hz, 0.4H), 7.18-7.24 (m, 2H), 7.26-7.32 (m, 3H).

¹³C NMR (CDCl₃, 125.8 MHz): δ 15.6 (CH₃), 15.8 (CH₃), 16.0 (CH₃), 16.3 (CH₃), 23.48 (CH₃), 23.50 (CH₃), 23.51 (CH₃), 25.38 (CH₂), 25.43 (CH₂), 27.25 (CH₂), 27.27 (CH₂), 27.5 (CH₂), 27.6 (CH₂), 28.4 (CH₂), 28.6 (CH₂), 29.7 (CH₂), 29.8 (CH₂), 30.8 (CH₂), 30.87 (CH₂), 30.88 (CH₂), 31.0 (CH₂), 32.2 (CH₂), 32.4 (CH₂), 35.8 (CH₂), 36.4 (CH₂), 37.5 (CH), 37.6 (CH), 37.7 (CH), 37.8 (CH), 37.83 (CH), 37.9 (CH), 38.0 (CH), 38.3 (CH), 69.71 (CH₂), 69.73 (CH₂), 72.72 (CH₂), 72.73 (CH₂), 103.0 (CH), 103.1 (CH), 106.1 (CH), 106.2 (CH), 120.97 (CH), 121.02 (CH), 121.1 (CH), 121.2 (CH), 126.3 (CH), 126.4 (CH), 128.37 (CH), 128.43 (CH), 128.93 (CH), 129.0 (CH), 133.89 (C), 133.91 (C), 133.95 (C), 133.98 (C), 138.34 (C), 138.36 (C), 145.06 (CH), 145.09 (CH), 146.38 (CH), 146.42 (CH).

### Compound 35. 1-(tert-butyl)-4-(2-methyl-4-phenethoxybut-3-en-1-yl)benzene

Following the procedure described for compound 25 and starting from compound 22 (8 g, 34.4 mmol) and 2-phenylethanol (10.5 g, 86.1 mmol), the title compound (3.19 g, 9.9 mmol) was isolated by distillation (bp 155° C., 2 Pa) in 29% yield as a colorless liquid (E/Z=44:56).

¹H NMR (CDCl₃, 500 MHz): δ 0.95 (d, J=6.7 Hz, 1.7H), 0.97 (d, J=6.7 Hz, 1.3H), 1.28 (s, 5H), 1.29 (s, 4H), 2.33 (septet, J=7.1 Hz, 0.44H), 2.48 (dd, J=7.6, 13.4 Hz, 1H), 2.58 (overlapping dd, J=7.0, 13.4 Hz, 1H), 2.82 (t, J=7.1 Hz, 1.12H), 2.90 (t, J=7.1 Hz, 0.88H), 2.87-2.97 (m, 0.56H), 3.73-3.88 (m, 2H), 4.25 (dd, J=6.3, 9.2 Hz, 0.56H), 4.74 (dd, J=8.1, 12.7 Hz, 0.44H), 5.84 (d, J=6.3 Hz, 0.56H), 6.15 (d, J=12.7 Hz, 0.44H), 7.04-7.10 (m, 2H), 7.17-7.23 (m, 3H), 7.24-7.32 (m, 4H).

¹³C NMR (CDCl₃, 125.8 MHz): δ 20.7 (CH₃), 21.1 (CH₃), 30.7 (CH), 31.41 (CH₃), 31.42 (CH₃), 34.29 (C), 34.31 (C), 34.7 (CH), 35.7 (CH₂), 36.7 (CH₂), 43.2 (CH₂), 44.2 (CH₂), 69.7 (CH₂), 72.7 (CH₂), 110.3 (CH), 113.2 (CH), 124.7 (CH), 124.9 (CH), 126.29 (CH), 126.34 (CH), 128.35 (CH), 128.40 (CH), 128.85 (CH), 128.90 (CH), 128.91 (CH), 129.0 (CH), 137.7 (C), 138.0 (C), 138.3 (C), 138.4 (C), 143.7 (CH), 145.1 (CH), 148.2 (C), 148.4 (C).

Compound 36. 1-ethoxynon-1-ene (comparative example corresponding to Example V of US20040013779): The diethyl acetal of nonanal (15 g, 69.3 mmol) and KHSO₄ (0.19 g, 1.37 mmol) were added to a 35 ml, round-bottomed flask equipped with a distillation head and nitrogen bubbler. The mixture was heated at 150° C. (oil bath) for 2 h while distilling out EtOH. The mixture was allowed to cool then placed under vacuum (67 Pa) and heated (120° C. oil bath). The vacuum was progressively lowered to 3 Pa and the reaction mixture distilled. Factions rich in the enol ether were combined and subjected to silica gel flash chromatography (hexane/EtOAc 100:0 to 95:5) to afford 1.7 g (10.0 mmol, 14% yield) of the title compound as a colorless oil (E/Z=50:50).

¹H NMR (C₆D₆, 600 MHz): δ 0.88 and 0.90 (overlapping t, J=7.0 Hz, 3H), 0.99 (t, J=7.1 Hz, 1.5H), 1.06 (t, J=7.1 Hz, 1.5H), 1.20-1.32 (m, 7H), 1.32-1.39 (m, 2H), 1.45 (pentet, J=7.5 Hz, 1H), 1.91 (q, J=7.4 Hz, 1H), 2.33 (q, J=7.4 Hz, 1H), 3.42 and 3.45 (overlapping q, J=7.1 Hz, 2H), 4.43 (q, J=6.9 Hz, 0.5H), 4.79 (dt, J=12.6, 7.4 Hz, 0.5H), 5.83 (d, J=6.3 Hz, 0.5H), 6.28 (d, J=12.6 Hz, 0.5H).

¹³C NMR (C₆D₆, 150.9 MHz): δ 14.3 (CH₃), 14.9 (CH₃), 15.4 (CH₃), 23.08 (CH₂), 23.09 (CH₂), 29.4 (CH₂), 29.6 (CH₂), 29.7 (CH₂), 30.4 (CH₂), 31.4 (CH₂), 32.3 (CH₂), 64.3 (CH₂), 67.4 (CH₂), 103.8 (CH), 107.0 (CH), 145.2 (CH), 146.8 (CH).

Compound 37. 1-ethoxyundec-1-ene (comparative example corresponding to Example VIII of US20040013779): The diethyl acetal of undecanal (8 g, 32.7 mmol) and KHSO₄ (0.14 g, 1.0 mmol) were added to a 25 ml, round-bottomed flask equipped with a distillation head and vacuum pump. The mixture was heated under vacuum (450 mbar) at 150° C. (oil bath) for 2 h while progressively reducing the pressure to 18 mbar. The vacuum was then reduced to 3.3 Pa and the reaction mixture distilled. Factions rich in the enol ether were combined and subjected to silica gel flash chromatography (hexane/EtOAc 100:0 to 95:5) to afford 0.8 g (4.0 mmol, 12% yield) of the title compound as a colorless oil (E/Z=45:55).

¹H NMR (C₆D₆, 600 MHz): δ 0.90 and 0.91 (overlapping t, J=7.1 Hz, 3H), 0.99 (t, J=7.0 Hz, 1.65H), 1.07 (t, J=7.0 Hz, 1.35H), 1.20-1.34 (m, 11H), 1.34-1.40 (m, 2H), 1.46 (pentet, J=7.5 Hz, 1H), 1.93 (q, J=7.2 Hz, 0.9H), 2.33 (q, J=7.5 Hz, 1.1H), 3.43 and 3.45 (overlapping q, J=7.0 Hz, 2H), 4.44 (q, J=6.8 Hz, 0.55H), 4.79 (dt, J=12.6, 7.3 Hz, 0.45H), 5.83 (d, J=6.3 Hz, 0.55H), 6.29 (d, J=12.6 Hz, 0.45H).

¹³C NMR (C₆D₆, 150.9 MHz): δ 14.4 (CH₃), 14.9 (CH₃), 15.4 (CH₃), 23.1 (CH₂), 23.11 (CH₂), 24.6 (CH₂), 28.4 (CH₂), 29.5 (CH₂), 29.77 (CH₂), 29.80 (CH₂), 29.81 (CH₂), 30.0 (CH₂), 30.1 (CH₂), 30.4 (CH₂), 31.4 (CH₂), 32.32 (CH₂), 32.33 (CH₂), 64.2 (CH₂), 67.4 (CH₂), 103.7 (CH), 107.0 (CH), 145.2 (CH), 146.8 (CH).

Compound 38. 1-ethoxydodec-1-ene (comparative example corresponding to Example X of US20040013779): The diethyl acetal of dodecanal (15 g, 58.0 mmol) and KHSO₄ (0.24 g, 1.73 mmol) were added to a 35 ml, round-bottomed flask equipped with a distillation head and vacuum pump. The mixture was heated under vacuum (450 mbar) at 150° C. (oil bath) for 2 h while progressively reducing the pressure to 18 mbar. The vacuum was then reduced to 3.3 Pa and the reaction mixture distilled. Factions rich in the enol ether were combined and subjected to silica gel flash chromatography (hexane/EtOAc 100:0 to 95:5) to afford 1.35 g (6.3 mmol, 11% yield) of the title compound as a colorless oil (E/Z=10:90).

¹H NMR (C₆D₆, 600 MHz, Z-isomer): δ 0.90 (t, J=7.0 Hz, 3H), 1.00 (t, J=7.1 Hz, 3H), 1.20-1.34 (m, 12H), 1.34-1.41 (m, 2H), 1.46 (pentet, J=7.5 Hz, 2H), 2.33 (q, J=7.4 Hz, 2H), 3.43 (q, J=7.0 Hz, 2H), 4.43 (q, J=6.9 Hz, 1H), 5.82 (d, J=6.2 Hz, 1H).

¹³C NMR (C₆D₆, 150.9 MHz, Z-isomer): δ 14.4 (CH₃), 15.4 (CH₃), 23.1 (CH₂), 24.6 (CH₂), 29.77 (CH₂), 29.81 (CH₂), 30.0 (CH₂), 30.1 (CH₂), 30.15 (CH₂), 30.4 (CH₂), 32.3 (CH₂), 67.3 (CH₂), 107.0 (CH), 145.2 (CH).

### Example 2

Headspace Analysis from Fabric Softener Application Comprising Invention's Compounds of Formula (I)

A model liquid fabric softener was prepared by mixing a TEA-esterquat (Stepantex® VL 90A), 12.3 wt %, 10% aqueous calcium chloride, 0.4 wt %, Proxcel GXL, 0.04 wt % and deionized water, 87.2 wt %. The enol ethers (0.075 mmol) were weighed into a vial and dissolved in 0.25 mL of acetone. Liquid fabric softener (4.5 g) was added to the vial and the mixture shaken by hand to mix. Reference samples were prepared in the same manner using 0.075 mmol of each released volatile. The fabric softener samples were rinsed with deionized water into a 3 L beaker and the beaker was filled to a total volume of 1.5 L. Three, 5-g cotton swatches (ca. 12.5×12.5 cm, weight 270 g/m², item 403 from Testfabrics, West Pittston, PA) were added to the beaker and agitated by hand for 3 min. After an additional 2 min of standing, the swatches were retrieved, and excess water squeezed out by hand. The cloths were hung to dry overnight (15-16 h) at rt. The swatches then were subjected to dynamic headspace analysis.

Each swatch was placed inside a thermostatted (25° C.), headspace sampling cell (about 160 mL volume). Using an air-sampling pump, a constant flow of air (200 mL/min) was drawn through the sampling cell and then through a cartridge containing 100 mg of Tenax® (the waste cartridge). Prior to entering the sample cell, the air was drawn through a plug of active charcoal and then through a saturated NaCl solution to maintain a constant relative humidity of 75%. Headspace samples were collected after 1 and 2 hours by replacing the waste cartridge with a clean Tenax® cartridge for 15 min. The cartridges were thermally desorbed with a Gerstel TDU 3.5 with cryofocusing at −30° C. and desorbed into an Agilent 8890 gas chromatograph equipped with an HP1 capillary column (30 m, i.d. 0.25 mm, film 0.25 m) and coupled with Agilent 5977B mass spectrometer. TDU temperature settings for desorption: 40° C. to 70° C. (30° C./min) hold for 4 min then heat to 260° C. (400° C./min) and hold 5 min. CIS settings (Tenax® packed liner): cryofocus at −30° C. then heat at 12° C./sec to 300° C. and hold for 4 min (heater mode: standard). PTV inlet settings: pressure 7.7 psi, total flow 99 ml/min, septum purge flow 3 ml/min on standard flow mode. The inlet mode was set to solvent vent with a purge flow to split vent set at 95 ml/min and the vent flow at 50 ml/min. The GC oven temperature profile was 52° C. to 110° C. at 20° C./min (hold 2 min) then ramped to 210° C. (20 C/min). The amount of each fragrance volatile collected (reported as ng/L of air) was determined using external standard calibrations of the respective chemicals. At least five acetone solutions were prepared with concentrations of the analytes ranging from 0.05 g/L to 5 g/L. The solutions were injected (0.2 L) onto Tenax® cartridges and desorbed as described above. Each solution was analyzed in triplicate. Calibration curves were forced through the origin.

TABLE 1
Dynamic headspace concentrations (ng/L) of perfumery raw materials obtained from
line-dried cotton treated with fabric softener containing enol ether profragrances
compared to their respective references (data for the 60-75 and 120-135 min
headspace samples and standard deviations).

| | | 60 min sample | | 120 min sample | |
|---|---|---|---|---|---|
| | perfumery raw material | profragrance | reference | profragrance | reference |
| Cmpd 1 | undecanal | 195 ± 21.1 | 3.1 ± 0.03 | 240 ± 4.9 | 4.4 ± 1.1 |
| | 2-phenylethyl formate | 128 ± 11.0 | 0.2 ± 0.1 | 125 ± 9.3 | 0.2 ± 0.04 |
| | 2-phenylethanol | 18.4 ± 1.6 | 4.4 ± 1.4 | 20.9 ± 1.0 | 7.5 ± 1.5 |
| Cmpd 2 | dodecanal | 448 ± 49.1 | 117 ± 13.2 | 479 ± 37.4 | 145 ± 38.0 |
| | 2-phenylethyl formate | 187 ± 15.4 | <1 | 174 ± 7.9 | <1 |
| | 2-phenylethanol^{a} | 54.8 ± 8.6 | — | 61.7 ± 8.7 | — |
| Cmpd 4 | phenylacetaldehyde | 47.5 ± 4.8 | 12.1 ± 2.9 | 48.0 ± 7.6 | 14.9 ± 1.6 |
| | 2-phenylethyl formate | 47.8 ± 9.6 | 2.6 ± 2.1 | 49.0 ± 11.0 | 3.6 ± 2.0 |
| | 2-phenylethanol | 25.9 ± 2.9 | 4.7 ± 1.6 | 32.9 ± 4.82 | 6.3 ± 2.2 |
| Cmpd 5 | 2-phenylpropanal | 565 ± 199 | 4.8 ± 3.5 | 348 ± 103 | 6.0 ± 4.9 |
| | 2-phenylethyl formate | 135 ± 59.8 | <1 | 97.4 ± 50.0 | n.d.b |
| | 2-phenylethanol^{a} | 29.8 ± 9.2 | — | 26.5 ± 10.1 | — |
| Cmpd 7 | undecanal | 185 ± 18.4 | 35.3 ± 8.8 | 203 ± 34.2 | 35.7 ± 8.7 |
| | octyl formate | 37.2 ± 1.2 | n.d.^{b} | 31.2 ± 4.3 | n.d. |
| | octanol^{a} | 201 ± 30.2 | — | 248 ± 41.2 | — |
| Cmpd 9 | decanal | 399 ± 35.5 | 26.0 ± 5.4 | 346 ± 38.9 | 32.2 ± 13.2 |
| | 2-phenylethyl formate | 168 ± 17.8 | 0.9 ± 0.08 | 140 ± 32.9 | 2.1 ± 1.6 |
| | 2-phenylethanol | 27.6 ± 3.3 | 5.7 ± 0.8 | 24.9 ± 8.2 | 9.7 ± 2 |
| Cmpd 10 | decanal | 185 ± 3.6 | 11.5 ± 8.5 | 130 ± 3.6 | 8.2 ± 1.7 |
| | 2-phenoxyethyl formate | 113 ± 0.6 | 2.5 ± 0.9 | 100 ± 5.8 | 2.3 ± 0.8 |
| | 2-phenoxyethan-1-ol | 40.1 ± 10 | 10.2 ± 1.7 | 36.5 ± 3.8 | 9.7 ± 2.5 |
| Cmpd 11 | decanal | 296 ± 87.8 | 9.0 ± 2.7 | 302 ± 102.6 | 8.6 ± 6.7 |
| | 3,7-dimethyloctanyl | 110 ± 36.6 | n.d | 84.7 ± 25.6 | n.d |
| | formate | | | | |
| | 3,7-dimethyloctane | 22.8 ± 6.4 | n.d. | 28.4 ± 11.2 | n.d. |
| Cmpd 13 | undecanal | 132 ± 33.2 | 4.0 ± 0.5 | 155 ± 46.9 | 5.8 ± 1.9 |
| | 2-octanyl formate | 28.5 ± 15 | n.d | 29.9 ± 20.5 | n.d. |
| | 2-octanol | 46.6 ± 9.8 | 0.4 ± 0.15 | 56.6 ± 12.8 | 0.36 ± 0.04 |
| Cmpd 14 | octanal | 366 ± 42.6 | 9.2 ± 0.5 | 270 ± 14.8 | 8.4 ± 0.7 |
| | 2-phenylethyl formate | 148 ± 28.9 | 0.33 ± 0.03 | 167 ± 41.9 | 0.16 ± 0.03 |
| | 2-phenylethanol | 13.8 ± 1.4 | 4.5 ± 1.8 | 17.7 ± 1.7 | 1-.3 ± 2.7 |
| Cmpd 15 | octanal | 473 ± 45.6 | 6.2 ± 0.9 | 335 ± 41.5 | 5.9 ± 0.5 |
| | citronellyl formate | 184 ± 20.2 | 0.57 ± 0.29 | 163 ± 16.2 | 0.5 ± 0.16 |
| | citronellol | 24.2 ± 1.1 | 1.9 ± 0.3 | 36.1 ± 2.2 | 2.0 ± 0.5 |
| Cmpd 25 | 2-methylundecanal | 222 ± 43.4 | 10.9 ± 2.4 | 216 ± 46.7 | 17.9 ± 3 |
| | 2-phenylethyl formate | 114 ± 15.1 | 0.7 ± 0.14 | 92.2 ± 69.2 | 0.7 ± 0.7 |
| | 2-phenylethanol | 9.2 ± 3.94 | 5.8 ± 5.8 | 11.1 ± 2.1 | 6.4 ± 0.88 |
| Cmpd 26 | 2-methylundecanal | 355 ± 49.1 | 7.3 ± 2.0 | 365 ± 91.3 | 10.5 ± 0.8 |
| | 2-phenoxyethyl formate | 209 ± 26.8 | 1.9 ± 0.6 | 252 ± 18.1 | 2.3 ± 0.6 |
| | 2-phenoxyethan-1-ol | 28 ± 4.6 | 5 ± 0.4 | 37.2 ± 6.2 | 6.5 ± 1.4 |
| Cmpd 27 | 2-methylundecanal | 474 ± 97.5 | 16.1 ± 3.6 | 346 ± 128 | 26.9 ± 7.5 |
| | cis-3-hexenyl formate | 24.4 ± 6.9 | n.d. | 19.5 ± 8.7 | n.d. |
| | cis-3-hexen-1-ol | 69.4 ± 44.9 | n.d. | 47.5 ± 35.2 | n.d. |
| Cmpd 29 | 2-methylundecanal | 465 ± 54.4 | 49.6 ± 7.5 | 446 ± 54.4 | 81.4 ± 23.7 |
| | 3-octanyl formate | 118 ± 31.6 | 12.8 ± 1.8 | 104 ± 32 | 11.6 ± 1.5 |
| | 3-octanol | 99 ± 38.4 | n.d. | 91.5 ± 28.3 | n.d. |
| Cmpd 30 | 10-undecanal | 222 ± 29.5 | 14.1 ± 2.5 | 213 ± 29.5 | 17.9 ± 6.0 |
| | 2-phenylethyl formate | 112 ± 23.9 | 0.3 ± 0.1 | 97.7 ± 8.9 | 0.8 ± 0.6 |
| | 2-phenylethanol | 15.9 ± 2.6 | 3.9 ± 0.7 | 16.2 ± 3.4 | 6.2 ± 2.1 |
| Cmpd 34 | 3-(4-methylcyclohex-3- | 58.1 ± 5.3 | 2.6 ± 0.5 | 78.4 ± 5.3 | 5.3 ± 0.2 |
| | en-1-yl)butanal | | | | |
| | 2-phenylethyl formate | 111 ± 13.0 | 0.3 ± 0.1 | 99.6 ± 21.1 | 0.4 ± 0.2 |
| | 2-phenylethanol | 19.7 ± 1.8 | 16.1 ± 8.2 | 23.7 ± 2.9 | 38.7 ± 44.3 |
| Cmpd 35 | 3-(4-(tert-butyl)phenyl)- | 103 ± 32.7 | 5.8 ± 2.5 | 128 ± 14.1 | 8.2 ± 2.5 |
| | 2-methylpropanal | | | | |
| | 2-phenylethyl formate | 129 ± 6.4 | 1.1 ± 0.6 | 145 ± 25.9 | 1.1 ± 0.7 |
| | 2-phenylethanol | 12.4 ± 1.1 | 1.85 ± 0.1 | 16.2 ± 1.3 | 3.0 ± 0.3 |

^{a}This PRM was not added to the reference fabric softener.
^{b}Not detected.

These data show that, when applied to cotton fabric from a liquid fabric softener application, the compounds of formula (I) release considerably more perfumery ingredients (aldehydes, formate esters and alcohols) than the corresponding reference samples. This demonstrates that the compounds of the invention produced the desired slow-release effect.

### Example 3

Headspace Analysis from Liquid Laundry Detergent Application Comprising Invention's Compounds of Formula (I)

The profragrance enol ethers (0.075 mmol) were weighed into a vial and dissolved in 0.25 mL of acetone. Liquid laundry detergent (4.5 g, Tide Simply Free and Sensitive) was added to the vial and the mixture shaken by hand to mix. The liquid laundry detergent was diluted into 1.0 L of deionized water in a large beaker. Three, 5-g cotton swatches (ca. 12.5×12.5 cm, weight 270 g/m², item 403 from Testfabrics, West Pittston, PA) were added to the beaker and agitated by hand for 2 minutes and then allowed to soak in the detergent solution for an additional 13 min. The swatches were removed, and excess liquid squeezed out by hand. In a second beaker, the swatches were placed in 500 L of deionized water, separated by hand and allowed to soak for 2 ma. The swatches then were removed individually, and the excess liquid squeezed out by hand. Reference samples were prepared in the same manner using 0.075 mmol of each released volatile. The cloths were hung to dry overnight (15-16 h) at rt. The swatches then were subjected to dynamic headspace analysis as described in Example 2.

TABLE 2
Dynamic headspace concentrations (ng/L) of perfumery raw materials obtained from
line-dried cotton treated with liquid laundry detergent containing enol ether profragrances
compared to their respective references (data for the 60-75 and 120-135 min headspace
samples and standard deviations).

| | | 60 min sample | | 120 min sample | |
|---|---|---|---|---|---|
| | perfumery raw material | profragrance | reference | profragrance | reference |
| Cmpd 25 | 2-methylundecanal | 178 ± 11.7 | 5.9 ± 0.81 | 178 ± 24.4 | 7.7 ± 0.9 |
| | 2-phenylethyl formate | 106 ± 18.2 | 1.5 ± 0.6 | 75.4 ± 11.5 | 0.8 ± 0.3 |
| | 2-phenylethanol | 12.1 ± 1.97 | 2.5 ± 0.75 | 11.5 ± 2.3 | 2.8 ± 0.2 |
| Cmpd 29 | 2-methylundecanal | 119 ± 47.3 | 6.3 ± 0.1 | 150 ± 2.4 | 14.9 ± 2.2 |
| | 3-octyl formate | 16.2 ± 1.6 | n.d. | 12.2 ± 3.6 | n.d. |
| | 3-octanol | 10.9 ± 1.3 | n.d. | 8.2 ± 2.5 | n.d. |
| Cmpd 30 | 10-undecenal | 132 ± 60.9 | 9.4 ± 1.3 | 115 ± 16.0 | 10.2 ± 0.3 |
| | 2-phenylethyl formate | 75.2 ± 42.5 | 2.8 ± 1.6 | 55.0 ± 7.1 | 0.5 ± 0.1 |
| | 2-phenylethanol | 9.9 ± 3.0 | 5.7 ± 1.2 | 9.2 ± 0.8 | 2.7 ± 0.6 |
| Cmpd 34 | 3-(4-methylcyclohex- | 17.3 ± 2.1 | 1.5 ± 0.3 | 17.6 ± 10.6 | 1.9 ± 0.6 |
| | 3-en-1-yl)butanal | | | | |
| | 2-phenylethyl formate | 31.7 ± 7.6 | 1.6 ± 0.6 | 24.3 ± 12.5 | 1.1 ± 0.2 |
| | 2-phenylethanol | 6.3 ± 2.3 | 4.3 ± 2.4 | 6.4 ± 4.6 | 6.7 ± 2.3 |
| Cmpd 35 | 3-(4-(tert- | 34.3 ± 8.8 | 2.3 ± 1.3 | 32.3 ± 8.1 | 2.9 ± 1.2 |
| | butyl)phenyl)-2- | | | | |
| | methylpropanal | | | | |
| | 2-phenylethyl formate | 36.9 ± 7.4 | 0.5 ± 0.1 | 23.2 ± 2.1 | 0.32 ± .01 |
| | 2-phenylethanol | 4.4 ± 0.27 | 4.2 ± 1.3 | 3.3 ± 0.3 | 2.7 ± 3.3 |

^{a}Not detected.

These data show that, when applied to cotton fabric from a liquid laundry detergent application, the compounds of formula (I) release considerably more perfumery ingredients (aldehydes, formate esters and alcohols) than the corresponding reference samples. This demonstrates that the compounds of the invention produced the desired slow-release effect.

### Example 4

Headspace Analysis from Fabric Softener Application Comprising Invention's Compounds of Formula (I) and Comparative Compounds

The use of compounds of formula (I) for prolonging or enhancing the perfuming effect of aldehydes were compared to compounds as described in US 2004/0013779 A1. The abilities of these compounds to provide a long-lastingness to the release profile of highly volatile perfumery aldehydes was measured by dynamic headspace analysis of cotton swatches that were rinsed with liquid fabric softener and air-dried for 16 hours. The experiments were performed as described in Example 2 and the data reported in Table 3.

TABLE 3
Dynamic headspace concentrations (ng/L) of perfumery aldehydes obtained from line-dried
cotton treated with fabric softener containing enol ethers compared to their respective references
(data for the 60-75 and 120-135 min headspace samples and standard deviations).

| | perfumery | 60 min sample | | 120 min sample | |
|---|---|---|---|---|---|
| | aldehyde | profragrance | reference | profragrance | reference |
| 1-ethoxynon-1-ene, | octanal | 12.1 ± 0.9 | 9.5 ± 0.7 | 10.1 ± 0.7 | 9.0 ± 0.3 |
| Comp 36 | | | | | |
| (Ex. V, US | | | | | |
| 2004/0013779 A1) | | | | | |
| Cmpd 14 | octanal | 366 ± 42.6 | 9.2 ± 0.5 | 270 ± 14.8 | 8.4 ± 0.7 |
| Cmpd 15 | octanal | 473 ± 45.6 | 6.2 ± 0.9 | 335 ± 41.5 | 5.9 ± 0.5 |
| 1-ethoxyundec-1-ene, | decanal | 12.8 ± 9.8 | 12.1 ± 3.3 | 20.0 ± 3.0 | 12.6 ± 1.9 |
| Cmpd 37 | | | | | |
| (Ex. VIII, US | | | | | |
| 2004/0013779 A1) | | | | | |
| Cmpd 9 | decanal | 399 ± 35.5 | 26.0 ± 5.4 | 346 ± 38.9 | 32.2 ± 13.2 |
| Cmpd 10 | decanal | 185 ± 3.6 | 11.5 ± 8.5 | 130 ± 3.6 | 8.2 ± 1.7 |
| Cmpd 11 | decanal | 296 ± 87.8 | 9.0 ± 2.7 | 302 ± 102.6 | 8.6 ± 6.7 |
| 1-ethoxydodec-1-ene, | undecanal | 6.3 ± 0.7 | 3.3 ± 1.2 | 9.4 ± 1.2 | 4.9 ± 0.3 |
| Cmpd 38 | | | | | |
| (Ex. X, US | | | | | |
| 2004/0013779 A1) | | | | | |
| Cmpd 1 | undecanal | 195 ± 21.1 | 3.1 ± 0.03 | 240 ± 4.9 | 4.4 ± 1.1 |
| Cmpd 7 | undecanal | 185 ± 18.4 | 35.3 ± 8.8 | 203 ± 34.2 | 35.7 ± 8.7 |
| Cmpd 13 | undecanal | 132 ± 33.2 | 4.0 ± 0.5 | 155 ± 46.9 | 5.8 ± 1.9 |

The Table above reports the amounts of perfumery aldehydes (octanal, decanal or undecanal) released from enol ethers of formula (I) compared to enol ethers reported in US 2004/0013779 A1. These data show that, when applied to cotton fabric from a liquid fabric softener application, the compounds of formula (I) release considerably more perfumery aldehydes than the comparative examples. Considering both time points measured, the compounds of formula (I) released 30 to 39-fold higher levels of octanal, 6.5 to 31-fold higher levels of decanal and 16 to 31-fold higher levels of undecanal. This demonstrates that the compounds of the invention produced the desired slow-release effect while the comparative examples did not. The comparative examples produced levels of aldehydes that were so similar to the respective reference samples that the comparative examples could not find practical use as slow-release agents of perfumery aldehydes for the purpose of prolonging or enhancing the perfuming effect of these aldehydes.

### Example 5

### Hydrolysis of Compounds According to Formula (I) and Comparative Compounds

The acid-catalyzed hydrolysis of Compounds 1, 2, 3 and 25 were measured and compared to the hydrolysis of enol ethers report in WO2019243501. Each enol ether was dissolved in a 4:1 THF/1 M HCl mixture and the percent remaining over time measured relative to an internal standard.

Into a 15 mL vial were added 125 mg of enol ether, 60 mg of hexadecane and 10 mL of THE (purged with N₂ and containing 2500 ppm of BHT). After mixing, 2 mL of this solution were removed with a volumetric pipet and used to obtain the time zero measurement. 2 mL of 1 M HCl were mixed with the remaining 8 mL of the THF solution. This mixture was divided into 5 mL vials (1 mL per vial). The vials were gently topped with nitrogen and fitted with screw caps around which parafilm was wrapped. The vials were stored at room temperature until analyzed. For analyses, 2 mL of ethyl acetate was added to a vial and mixed. After phase separation, the top phase was collected and washed with saturated sodium carbonate (1 mL). Samples of the organic phase were analyzed by GC-FID. For the time zero sample, 0.5 mL of deionized water was added to the 2 mL taken from the original THE solution. 1 mL of this solution was added to a 5 mL vial and diluted with 2 mL of ethyl acetate and mixed. The top phase was collected and washed with saturated sodium carbonate (1 mL). The top phase then was analyzed by GC-FID. The percent of enol ether remaining was determined by dividing the peak area ratio of the enol ether to internal standard by the ratio measured at time zero.

TABLE 4
Hydrolysis of enol ethers in THF/1M HCl (80:20 v/v)

| | | percent remaining | | | | | |
|---|---|---|---|---|---|---|---|
| entry | compd | initial | 5 h | 1 d | 2 d | 3 d | 7 d |
| 1 | Compound 1 | 100 | 12.8 | 0 | 0 | 0 | 0 |
| 2 | Compound 2 | 100 | 16.3 | 0 | 0 | 0 | 0 |
| 3 | Compound 3 | 100 | 19.9 | 0 | 0 | 0 | 0 |
| 4 | Compound 25 | 100 | 35.6 | 4.6 | 0 | 0 | 0 |
| 5 | Compound 27 | 100 | 29.8 | — | — | — | — |
| 6 | Compound 29 | 100 | 3.7 | — | — | — | — |
| 7 | (2-phenethoxyvinyl) | 100 | — | 95 | 90 | 83.7 | 66.4 |
| | benzene (Ex. 20, | | | | | | |
| | WO 2019/243501) | | | | | | |
| 8 | (2-(((Z)-hex-3-en- | 100 | — | — | — | 79.5 | 57.3 |
| | 1-yl)oxy)vinyl) | | | | | | |
| | benzene (Ex. 19, | | | | | | |
| | WO 2019/243501) | | | | | | |
| 9 | (2-(octan-3-yloxy) | 100 | — | 84.9 | 72.0 | 61.7 | 33.6 |
| | vinyl)benzene | | | | | | |
| | (Ex. 22, | | | | | | |
| | WO 2019/243501) | | | | | | |
| 10 | 1-(2-(((Z)-hex-3-en- | 100 | — | 89.1 | 80.2 | 72.4 | 52.5 |
| | 1-yl)oxy)vinyl)-4- | | | | | | |
| | methoxybenzene | | | | | | |
| | (covered by WO | | | | | | |
| | 2019/243501) | | | | | | |

The Table above shows the rate of hydrolysis for enol ethers of formula (I) prepared from 2-phenylethanol (Z)-3-hexen-1-ol and 3-octanol compared to enol ethers prepared from the same alcohols but derived from phenyl acetaldehyde derivatives as reported in WO2019243501. The table shows that the enol ethers of formula (I) hydrolyzed more rapidly when treated with 1M HCl (pH 0) than the corresponding enol ethers as covered in WO2019243501. Therefore, the enol ethers of formula (I) can release the perfuming ingredient of formula (IV) more rapidly and at a different time point than enol ethers reported in WO2019243501 under use conditions that promote release by hydrolysis (e.g., high humidity climates)

### Example 6

### Preparation of a Perfume Oil

A non-limiting example of a typical perfume oil is prepared by admixing the following perfuming co-ingredients:

### Ingredients Weight %

Ethyl 2-methylbutanoate 0.16
Hexyl acetate 0.37
Limonene 1.67
2,6-Dimethyl-7-octen-2-ol 0.94
2-Phenylethanol 2.15
Linalool 0.73
(2RS,4SR/4RS)-4-Methyl-2-(2-methyl-1-propen-1-yl)tetrahydro-2H-pyran 0.30
Ethyl 2-methyl-1,3-dioxolane-2-acetate 0.32
Benzyl acetate 2.46
Allyl heptanoate 0.38
alpha-Terpineol 0.88
3,7-Dimethyl-6-octen-1-ol 0.55
4-Methoxybenzaldehyde 1.00
(E)-4-Methyl-3-decen-5-ol 0.37
[cis/trans-4-(2-Propanyl)cyclohexyl]methanol 0.47
1-Methoxy-4-[(1E)-1-propen-1-yl]benzene 0.15
(1RS,2RS/2SR)-2-(2-Methyl-2-propanyl)cyclohexyl acetate 1.95
1,1-Dimethyl-2-phenylethyl acetate 0.95
Tricyclo[5.2.1.0²˜]dec-3/4-en-8-yl acetate 3.34
Allyl 3-cyclohexylpropanoate 0.26
3-(4-Isopropylphenyl)-2-methylpropanal 8.18
(3E)-3-Methyl-4-(2,6,6-trimethyl-2-cyclohexen-1-yl)-3-buten-2-one and
(1E)-1-(2,6,6-trimethyl-2-cyclohexen-1-yl)-1-penten-3-one 1.13
2-Phenoxyethyl 2-methylpropanoate 5.38
Tricyclo[5.2.1.0(2,6)]dec-3/4-en-8-yl propanoate 2.32
5-Heptyldihydro-2(3H)-furanone 2.30
2/3-Methylbutyl salicylate 1.42
(3Z)-3-Hexen-1-yl salicylate 0.31
1-(2,3,8,8-Tetramethyl-1,3,4,5,6,7-hexahydronaphthalen-2-yl)ethanone 16.03
Hexyl 2-hydroxybenzoate 5.04
(2E)-2-Benzylideneoctanal 21.22
(−)-(3aR,5aS,9aS,9bR)-3a,6,6,9a-Tetramethyldodecahydronaphtho[2,1-b]furan 0.27
Habanolide® 4.78
Exaltolide® 3.82
Benzyl 2-hydroxybenzoate 3.01
Dipropylene glycol 5.39
   Total: 100

### Example 7

Preparation of all-Purpose Cleaner Formulations Comprising an Invention's Compound of Formula (I)

A typical all-purpose cleaner formulation is listed in Table 5. A perfumed all-purpose cleaner is prepared by adding, under gentle shaking, a perfume oil of Example 6 (0.3 to 0.8% by weight relative to the total weight of the all-purpose cleaner) and at least one of the invention's compounds of formula (I) (0.05 to 0.8% by weight relative to the total weight of the all-purpose cleaner) into the unperfumed all-purpose cleaner formulation of Table 5.

TABLE 5
Composition of a typical unperfumed all-purpose cleaner formulation

| | Amount |
|---|---|
| Ingredients | [wt %] |
| Ethoxylated alcohol (C₉-C₁₁, 8 EO) ⁽¹⁾ | 20.0 |
| Sodium dodecyl benzene sulfonate ⁽²⁾ | 16.0 |
| Sodiumcumene sulfonate ⁽³⁾ | 8.0 |
| Methyl chloro isothiazolinone/ | 0.8 |
| methyl isothiazolinone 3.3:1 ⁽⁴⁾ | |
| Deionized water | 55.9 |

⁽¹⁾ Neodol ® 91-8; origin: Shell Chemicals
⁽²⁾ Biosoft ® D-40; origin: Stepan
⁽³⁾ Stepanate ® SCS; origin: Stepan
⁽⁴⁾ Kathon ® CG; origin: Dow Chemicals

### Example 8

### Preparation of a Solid Detergent Comprising an Invention's Compound of Formula (I)

The chassis of a model powder detergent base comprises sodium sulfate, sodium carbonate, sodium dodecylbenzensulfonate, sodium silicate, zeolite, C₁₂₋₁₅ pareth-7, bentonite, perborate, TAED, citric acid, sodium acrylic acid/MA co-polymer, sodium carbonate peroxide, tetrasodium etidronate, sodium chloride, sodium bicarbonate, cellulose gum, disodium anilinomorpholinotriazinylaminostilbenesulfonate, phenylpropyl dimethicone, enzyme, dye. A typical unperfumed model powder detergent base is composed as listed in Table 6. A perfumed solid detergent is prepared by adding under gentle shaking the perfume oil of Example 6 (0.3 to 0.6% by weight, relative to the total weight of the solid detergent) and at least one of the invention's compounds of formula (I) (0.15% by weight, relative to the total weight of the solid detergent).

TABLE 6
Composition of a typical unperfumed
powder detergent

| | Amount |
|---|---|
| Ingredients | [wt %] |
| Anionic surfactant | 5-25% |
| Non-ionic surfactant | 2-15% |
| Builder | 20-50% |
| Percarborate | 5-20% |
| TAED | 1-8% |
| Polymer | 3-10% |
| Optical brightener | 0.1-0.5% |
| Enzyme, Dye | <2% |

### Example 9

### Preparation of a Bleach-Free Solid Detergent Comprising an Invention's Compound of Formula (I)

Typical bleach-free powder detergent formulations are composed of sodium sulfate, sodium carbonate, sodium dodecylbenzensulfonate, sodium silicate, zeolite, C₁₂₋₁₅ pareth-7, bentonite, citric acid, sodium acrylic acid/MA co-polymer, sodium carbonate peroxide, tetrasodium etidronate, sodium chloride, sodium bicarbonate, cellulose gum, disodium anilinomorpholinotriazinylaminostilbenesulfonate, phenylpropyl dimethicone, enzyme, dye. A typical unperfumed model powder detergent base is composed as listed in Table 7. A perfumed bleach-free solid detergent is prepared by adding under gentle shaking the perfume oil of Example 6 (0.3 to 0.6% by weight, relative to the total weight of the bleach-free solid detergent) and at least one of the invention's compounds of formula (I) (0.15% by weight, relative to the total weight of the bleach-free solid detergent).

TABLE 7
Composition of a typical unperfumed
bleach-free powder detergent

| | Amount |
|---|---|
| Ingredients | [wt %] |
| Anionic surfactant | 5-20% |
| Non-ionic surfactant | 3-12% |
| Builder | 20-65% |
| Polymer | 3-10% |
| Optical brightener | 0.1-0.5% |
| Enzyme, Dye | <1% |

### Example 10

### Preparation of a Hand Dishwash Formulation Comprising an Invention's Compound of Formula (I)

A typical hand dishwash formulation is listed in Table 8. Water, sodium hydroxide and diethanolamide are mixed. Then linear alkylbenzene sulfonic acid is added. After neutralization, the remaining ingredients are added. The pH is checked (7-8) and adjusted if necessary. A perfumed hand dishwash is prepared by adding under gentle shaking the perfume oil of Example 6 (0.4 to 0.8% by weight, relative to the total weight of the hand dishwash formulation) and at least one of the invention's compounds of formula (I) (0.02 to 0.5% by weight relative to the total weight of the unperfumed formulation) into the unperfumed hand dishwash formulation of Table 8.

TABLE 8
Composition of a typical unperfumed
hand dishwash formulation

| | Amount |
|---|---|
| Ingredients | [wt %] |
| Linear alkylbenzene sulfonic acid ⁽¹⁾ | 20.0 |
| Diethanolamine ⁽²⁾ | 3.5 |
| Sodium hydroxide (50%) ⁽³⁾ | 3.4 |
| Secondary alcohol ethoxolate ⁽⁴⁾ | 2.5 |
| Sodium xylene sulfonate | 6.3 |
| Deionized water | 64.3 |

⁽¹⁾ Biosoft ® S-118; origin: Stepan
⁽²⁾ Ninol ® 40-CO; origin: Stepan
⁽³⁾ Stepanate ® SXS; origin: Stepan
⁽⁴⁾ Tergitol ® 15-S-9; origin: Dow Chemicals

### Example 11

### Preparation of a Transparent Isotropic Shampoo Formulation Comprising an Invention's Compound of Formula (I)

A typical unperfumed transparent isotropic shampoo formulation is listed in Table 9. The unperfumed shampoo formulation is prepared by dispersing Polyquaternium-3 in water. The remaining ingredients of Phase A are mixed separately by addition of one after the other while mixing well after each adjunction. This pre-mix is added to the Polyquaternium-10 dispersion and mixed for another 5 min. Then, the premixed Phase B and the premixed Phase C are added (Monomuls® 90L-12 is heated to melt in Texapon® NSO IS) while agitating. Phase D and Phase E are added while agitating. The pH is adjusted with a citric acid solution to 5.5-6.0 to give the unperfumed shampoo formulation listed in Table 9. The perfumed shampoo formulation is obtained by adding, under gentle shaking, the perfume oil of Example 6 (0.1 to 0.8% by weight relative to the total weight of the unperfumed shampoo formulation) and at least one of the compounds of formula (I) (0.05 to 0.50 by weight relative to the total weight of the unperfumed shampoo formulation) into the unperfumed shampoo formulation listed in Table 9.

TABLE 9
Composition of a typical unperfumed transparent
isotropic shampoo formulation

| | | Amount |
|---|---|---|
| Phase | Ingredients | [wt %] |
| A | Deionized water | 44.4 |
| | Polyquaternium-10 ⁽¹⁾ | 0.3 |
| | Glycerin 85% ⁽²⁾ | 1.0 |
| | DMDM Hydantoin ⁽³⁾ | 0.2 |
| B | Sodium laureth sulfate ⁽⁴⁾ | 28.0 |
| | Cocamidopropyl betaine ⁽⁵⁾ | 3.2 |
| | Disodium cocoamphodiacetate ⁽⁶⁾ | 4.0 |
| | Ethoxy (20) stearyl alcohol ⁽⁷⁾ | 1.0 |
| C | Sodium laureth sulfate ⁽⁴⁾ | 3.0 |
| | Glyceryl laureate ⁽⁸⁾ | 0.2 |
| D | Deionized water | 1.0 |
| | Sodium methylparaben ⁽⁹⁾ | 0.1 |
| E | Sodium chloride (10% aqueous solution) | 15.0 |
| | Citric acid (10% aqueous solution to | q.s. |
| | pH 5.5-6.0) | |

⁽¹⁾ Ucare ® Polymer JR-400; origin: Noveon
⁽²⁾ Origin: Brenntag Schweizerhall AG
⁽³⁾ Glydant ®; origin: Lonza
⁽⁴⁾ Texapon ® NSO IS; origin: Cognis
⁽⁵⁾ Tego ® Betain F 50; origin: Evonik
⁽⁶⁾ Amphotensid GB 2009; origin: Zschimmer & Schwarz
⁽⁷⁾ Brij ® S20; origin: Croda
⁽⁸⁾ Monomuls ® 90 L-12; origin: Gruenau GmbH
⁽⁹⁾ Nipagin Monosodium; origin: NIPA

### Example 12

### Preparation of a Pearly Shampoo Formulation Comprising an Invention's Compound of Formula (I)

A typical unperfumed pearly shampoo formulation is listed in Table 10. The unperfumed shampoo formulation is prepared by dispersing Tetrasodium EDTA, Guar hydroxypropyltrimonium chloride and Polyquaternium-10 in water. NaOH (10% aqueous solution, Phase B) is added once Phase A is homogeneous. Then, the premixed Phase C is added, and the mixture heated to 75° C. Phase D ingredients are added and mixed until the mixture is homogeneous. The mixture is cooled. At 45° C., Phase E ingredients are added while mixing. The final viscosity is adjusted with NaCl (25% aqueous solution) and a pH of 5.5-6.0 is adjusted with NaOH (10% aqueous solution). A perfumed pearly shampoo formulation is obtained by adding, under gentle shaking, the perfume oil of Example 6 (0.1 to 0.8% by weight relative to the total weight of the unperfumed shampoo formulation) and at least one of the compounds of formula (I) (0.05 to 0.5% by weight relative to the total weight of the unperfumed shampoo formulation) into the unperfumed pearly shampoo formulation listed in Table 10.

TABLE 10
Composition of a typical unperfumed pearly shampoo formulation

| | | Amount |
|---|---|---|
| Phase | Ingredients | [wt %] |
| A | Deionized water | 45.97 |
| | Tetrasodium EDTA ⁽¹⁾ | 0.05 |
| | Guar hydroxypropyl-trimonium chloride ⁽²⁾ | 0.05 |
| | Polyquaternium-10 ⁽³⁾ | 0.075 |
| B | NaOH (10% aqueous solution) | 0.30 |
| C | Ammonium lauryl sulfate ⁽⁴⁾ | 34.00 |
| | Ammonium laureth sulfate ⁽⁵⁾ | 9.25 |
| | Cocamidopropyl betaine ⁽⁶⁾ | 2.00 |
| | Dimethicone (&) C₁₂₋₁₃ pareth-4 (&) | 2.50 |
| | C₁₂₋₁₃ pareth-23 (&) salicylic acid ⁽⁷⁾ | |
| D | Cetyl alcohol ⁽⁸⁾ | 1.20 |
| | Cocamide MEA ⁽⁹⁾ | 1.50 |
| | Glycol distearate ⁽¹⁰⁾ | 2.00 |
| E | Methylchloroisothiazolinone & | 0.10 |
| | methylisothiazolinone ⁽¹¹⁾ | |
| | D-Panthenol 75% ⁽¹²⁾ | 0.10 |
| | Deionized water | 0.30 |
| F | Sodium chloride (25% aqueous solution) | 0.60 |

⁽¹⁾ EDETA ® B Powder; origin: BASF
⁽²⁾ Jaguar ® C14 S; origin: Rhodia
⁽³⁾ Ucare ® Polymer JR-400; origin: Noveon
⁽⁴⁾ Sulfetal ® LA B-E; origin: Zschimmer & Schwarz
⁽⁵⁾ Zetesol ® LA; origin: Zschimmer & Schwarz
⁽⁶⁾ Tego ® Betain F 50; origin: Evonik
⁽⁷⁾ Xiameter ® MEM-1691; origin: Dow Corning
⁽⁸⁾ Lanette ® 16; origin: BASF
⁽⁹⁾ Comperlan ® 100; origin: Cognis
⁽¹⁰⁾ Cutina ® AGS; origin: Cognis
⁽¹¹⁾ Kathon ® CG; origin: Rohm & Haas
⁽¹²⁾ D-Panthenol; origin: Roche

### Example 13

### Preparation of a Rinse-Off Hair Conditioner Formulation Comprising an Invention's Compound of Formula (I)

A typical unperfumed rinse-off hair conditioner formulation is listed in Table 11. The unperfumed rinse-off hair conditioner formulation is prepared by mixing the ingredients of Phase A until a uniform mixture was obtained. Tylose® is allowed to completely dissolve. Then the mixture is heated to 70-75° C. The ingredients of Phase B are combined and melted at 70-75° C. Then the ingredients of Phase B are added to Phase A with good agitation, and the mixing is continued until that the mixture has a temperature of 60° C. Then, the ingredients of Phase C are added while agitating and keeping mixing until the mixture cooled to 40° C. The pH is adjusted with a citric acid solution to 3.5-4.0. A perfumed rinse-off hair conditioner formulation is obtained by adding, under gentle shaking, the perfume oil of Example 6 (0.2 to 1.0% by weight relative to the total weight of the unperfumed conditioner formulation) and at least one of the compounds of formula (I) (0.05 to 0.5% by weight relative to the total weight of the unperfumed conditioner formulation) into the unperfumed rinse-off hair conditioner formulation listed in Table 11.

TABLE 11
Composition of a typical unperfumed rinse-off hair conditioner formulation

| | | Amount |
|---|---|---|
| Phase | Ingredients | [wt %] |
| A | Deionized water | 81.8 |
| | Behentrimonium chloride ⁽¹⁾ | 2.5 |
| | Hydroxyethylcellulose ⁽²⁾ | 1.5 |
| B | Cetearyl alcohol ⁽³⁾ | 4.0 |
| | Glyceryl stearate (and) PEG-100 stearate ⁽⁴⁾ | 2.0 |
| | Behentrimonium metho-sulfate (and) | 4.0 |
| | cetyl alcohol (and) butylene glycol ⁽⁵⁾ | |
| | Ethoxy (20) stearyl alcohol ⁽⁶⁾ | 1.0 |
| C | Amodimethicone (and) Trideceth-12 (and) | 3.0 |
| | Cetrimonium chloride ⁽⁷⁾ | |
| | Chlorhexidine digluconate (20% aqueous solution) ⁽⁸⁾ | 0.2 |
| D | Citric acid (10% aqueous solution tol pH 3.5-4.0) | q.s. |

⁽¹⁾ Genamin ® KDMP; origin: Clariant
⁽²⁾ Tylose ® H10 Y G4; origin: Shin Etsu
⁽³⁾ Lanette ® O; origin: BASF
⁽⁴⁾ Arlacel ® 165; origin: Croda
⁽⁵⁾ Incroquat ® Behenyl TMS-50-PA- (MH); origin: Croda
⁽⁶⁾ Brij ® S20; origin: Croda
⁽⁷⁾ Xiameter ® MEM-949; origin: Dow Corning
⁽⁸⁾ Origin: Alfa Aesar

### Example 14

### Preparation of a Structured Shower Gel Formulation Comprising an Invention's Compound of Formula (I)

A typical unperfumed structured shower gel formulation is listed in Table 12. A perfumed structured shower gel is prepared by adding, under gentle shaking, the perfume oil of Example 6 (0.1 to 1.5% by weight relative to the total weight of the structured shower gel) and at least one of the invention's compounds of formula (I) (0.05 to 0.5% by weight relative to the total weight of the structured shower gel) into the unperfumed structured shower gel formulation of Table 12.

TABLE 12
Composition of a typical unperfumed structured shower gel formulation

| | Amount |
|---|---|
| Ingredients | [wt %] |
| Deionized water | 49.35 |
| Tetrasodium EDTA ⁽¹⁾ | 0.05 |
| Acrylates co-polymer ⁽²⁾ | 6.00 |
| Sodium C₁₂₋₁₅ pareth sulfate ⁽³⁾ | 35.00 |
| Sodium hydroxide (20% aqueous solution) | 1.00 |
| Cocamidopropyl betaine ⁽⁴⁾ | 8.00 |
| Methylchloroisothiazolinone and methylisothiazolinone ⁽⁵⁾ | 0.10 |
| Citric acid (40% aqueous solution) | 0.50 |

⁽¹⁾ EDETA B powder; origin: BASF
⁽²⁾ Carbopol Aqua SF-1 polymer; origin: Noveon
⁽³⁾ Zetesol AO 328 U; origin: Zschimmer & Schwarz
⁽⁴⁾ Tego Betain F 50; origin: Goldschmidt
⁽⁵⁾ Kathon ® CG; origin: Rohm & Haas

### Example 15

### Preparation of a Transparent Shower Gel Formulation Comprising an Invention's Compound of Formula (I)

A typical unperfumed transparent shower gel formulation is listed in Table 13. A perfumed transparent shower gel is prepared by adding, under gentle shaking, the perfume oil of Example 6 (0.5 to 1.5% by weight relative to the total weight of the transparent shower gel) and at least one of the invention's compounds of formula (I) (0.05 to 0.5%0 by weight relative to the total weight of the transparent shower gel) into the unperfumed transparent shower gel formulation of Table 13.

TABLE 13
Composition of a typical unperfumed transparent
shower gel formulation

| | Amount |
|---|---|
| Ingredients | [wt %] |
| Deionized water | 52.40 |
| Tetrasodium EDTA ⁽¹⁾ | 0.10 |
| Sodium benzoate | 0.50 |
| Propylene glycol | 2.00 |
| Sodium C₁₂₋₁₅ pareth sulfate ⁽²⁾ | 35.00 |
| Cocamidopropyl betaine ⁽³⁾ | 8.00 |
| Polyquaternium-7 ⁽⁴⁾ | 0.20 |
| Citric acid (40% aqueous solution) | 1.00 |
| Sodium chloride | 0.80 |

⁽¹⁾ EDETA B powder; origin: BASF
⁽²⁾ Zetesol AO 328 U; origin: Zschimmer & Schwarz
⁽³⁾ Tego Betain F 50; origin: Goldschmidt
⁽⁴⁾ Merquat ® 550; origin: Lubrizol

### Example 16

### Preparation of a Milky Shower Gel Formulation Comprising an Invention's Compound of Formula (I)

A typical unperfumed milky shower gel formulation is listed in Table 14. A perfumed milky shower gel is prepared by adding, under gentle shaking, the perfume oil of Example 6 (0.1 to 1.5% by weight relative to the total weight of the milky shower gel) and at least one of the invention's compounds of formula (I) (0.05 to 0.5% by weight relative to the total weight of the milky shower gel) into the unperfumed milky shower gel formulation of Table 14.

TABLE 14
Composition of a typical unperfumed milky
shower gel formulation

| | Amount |
|---|---|
| Ingredients | [wt %] |
| Deionized water | 50.95 |
| Tetrasodium EDTA ⁽¹⁾ | 0.05 |
| Sodium benzoate | 0.50 |
| Glycerin (86% aqueous solution) | 3.50 |
| Sodium laureth sulfate ⁽²⁾ | 27.00 |
| Polyquaternium-7 ⁽³⁾ | 1.00 |
| Coco-betaine ⁽⁴⁾ | 6.00 |
| PEG-120 Methyl glucose trioleate ⁽⁵⁾ | 1.00 |
| Citric acid (40% aqueous solution) | 1.00 |
| Glycol distearate & laureth-4 & | 3.00 |
| cocamidopropyl betaine ⁽⁶⁾ | |
| Sodium chloride (20% aqueous solution) | 5.00 |
| PEG-40 hydrogenated castor oil (7) | 1.00 |

⁽¹⁾ EDETA ® B powder; origin: BASF
⁽²⁾ Texapon ® NSO IS; origin: Cognis
⁽³⁾ Merquat ® 550; origin: Lubrizol
⁽⁴⁾ Dehyton ® AB-30; origin: Cognis
⁽⁵⁾ Glucamate ® LT; origin: Lubrizol
⁽⁶⁾ Euperlan ® PK 3000 AM; origin: Cognis
⁽⁷⁾ Cremophor ® RH 40; origin: BASF.

1. A method to release from a precursor compound, compounds selected from the group consisting of
a) an aldehyde compound of formula 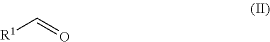
wherein
R¹ is a C₁₋₁₅ alkyl, C₃₋₁₅ alkenyl, C₃₋₁₅ cycloalkyl, C₅₋₁₅ cycloalkenyl or C₃₋₁₄ heterocycloalkyl group, each optionally substituted with one or more of a hydroxy, C₁₋₁₅ alkyl, C₂₋₁₅ alkenyl, C₁₋₁₅ alkoxy, C₂₋₁₅ alkenyloxy, C₃₋₁₅ cycloalkyl, C₅₋₁₅ cycloalkenyl, C₃₋₁₅ heterocycloalkyl, carboxylic acid, C₁₋₄ carboxylic ester, C₆₋₁₀ aryl and/or C₆₋₁₀ aryloxy group, each optionally substituted with one or more of a C₁₋₈ alkyl, C₁₋₈ alkoxy, hydroxy, carboxylic acid and/or C₁₋₄ carboxylic ester group;
b) a formate ester of formula 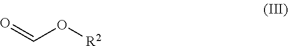
R² is a C₄₋₁₈ hydrocarbon group optionally comprising one or two oxygen atoms; provided that an ester functional group alpha to the formyloxy group is excluded and provided that R² does not comprise an allylic functional group;
c) an alcohol of formula 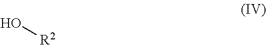
wherein R² has the same meaning as defined above;
wherein the precursor compound comprises a compound of formula (I) 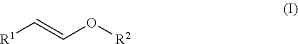
in the form of any one of its stereoisomers or a mixture thereof, and wherein R¹ and R² have the same meaning as defined above;
by exposing the precursor compound of formula (I) to an environment wherein the compound is oxidized.

2. The method according to claim 1, wherein the compound of formula (I) is a C₁₈-C₃₆ compound.

3. The method according to claim 1, wherein R² is a C₅₋₁₈ hydrocarbon group optionally comprising one or two oxygen atoms.

4. The method according to claim 1, wherein R² is a C₆₋₁₈ hydrocarbon group optionally comprising one or two oxygen atoms.

5. The method according to claim 1, wherein R² is a C₁₋₁₅ alkyl, C₂₋₁₅ alkenyl, C₃₋₁₅ cycloalkyl or C₅₋₁₅ cycloalkenyl group, each optionally substituted with one or more of a hydroxy, C₁₋₁₅ alkyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, hydroxy and/or carboxylic acid.

6. The method according to claim 1, wherein R¹ is a C₁₋₁₀ alkyl, C₃₋₁₀ alkenyl, C₃₋₁₁ cycloalkyl or C₅₋₁₁ cycloalkenyl group, each optionally substituted with one or more of a hydroxy, C₁₋₄ alkyl, C₁₋₄ alkoxy, C₃₋₈ cycloalkyl, C₅₋₈ cycloalkenyl, C₆ aryl and/or C₆ aryloxy group, each optionally substituted with one or more of a C₁₋₄ alkyl, hydroxy and/or C₁₋₄ alkoxy group.

7. The method according to claim 1, wherein the compound of formula (II), (III) and/or (IV) are a perfuming ingredient.

8. The method according to claim 1, wherein the environment wherein the compound is oxidized is air.

9. A method to confer, enhance, improve or modify the odor properties of a perfuming composition, the air surrounding the perfuming composition, a surface or a perfumed article, comprising adding to the composition, the air, or article, or contacting or treating the surface with an effective amount of at least one compound of formula (I) as defined in claim 1.

10. A method for intensifying or prolonging the diffusion effect of the characteristic fragrance of at least one aldehyde compound of formula (II), of at least one active formate ester of formula (III) and/or of at least one active alcohol of formula (IV) as defined in claim 1, on a surface or the air surrounding the perfuming composition, wherein the surface, or the air is treated with at least one compound (I) as defined in claim 1, or with a composition or article containing at least one compound (I), under conditions susceptible of allowing the release of at least one ketone or aldehyde formula (II), of at least one formate ester of formula (III) and/or of at least one alcohol of formula (IV) over time.

11. A perfuming composition comprising
i) at least one compound of formula (I), as defined in claim 1;
ii) at least one ingredient selected from the group consisting of a perfumery carrier and a perfumery base; and
iii) optionally at least one perfumery adjuvant.

12. A perfumed consumer product comprising at least one compound of formula (I), as defined in claim 1.

13. The perfumed consumer product according to claim 12, wherein the perfumery consumer product is a perfume, a fabric care product, a body-care product, a cosmetic preparation, a skin-care product, an air care product or a home care product.

14. The perfumed consumer product according to claim 13, wherein the perfumery consumer product is a fine perfume, a splash or eau de parfum, a cologne, a shave or after-shave lotion, a liquid or solid detergent optionally in the form of a pod or tablet, a fabric softener, a liquid or solid scent booster, a dryer sheet, a fabric refresher, an ironing water, a paper, a bleach, a carpet cleaner, a curtain-care product, a shampoo, a leave-on or rinse-off hair conditioner, a coloring preparation, a color-care product, a hair shaping product, a dental care product, a disinfectant, an intimate care product, a hair spray, skin cream or lotion, a vanishing cream, a deodorant or antiperspirant, a hair remover, a tanning or sun or after sun product, a nail product, a skin cleansing, a makeup, a perfumed soap, a shower or bath mousse, oil or gel, a foot/hand care product, a hygiene product, an air freshener, a “ready to use” powdered air freshener, a mold remover, a furnisher care, a wipe, a dish detergent or hard-surface detergent, a leather care product, a car care product.

15. A compound of formula 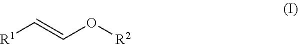
in the form of any one of its stereoisomers or a mixture thereof and wherein
R¹ comprises at least 6 carbon atoms and is a C₁₋₁₅ alkyl, C₃₋₁₅ alkenyl, C₃₋₁₅ cycloalkyl, C₅₋₁₅ cycloalkenyl or C₃₋₁₄ heterocycloalkyl group, each optionally substituted with one or more of a hydroxy, C₁₋₁₅ alkyl, C₂₋₁₅ alkenyl, C₁₋₁₅ alkoxy, C₂₋₁₅ alkenyloxy, C₃₋₁₅ cycloalkyl, C₅₋₁₅ cycloalkenyl, C₃₋₁₅ heterocycloalkyl, carboxylic acid, C₁₋₄ carboxylic ester, C₆₋₁₀ aryl and/or C₆₋₁₀ aryloxy group, each optionally substituted with one or more of a C₁₋₈ alkyl, C₁₋₈ alkoxy, hydroxy, carboxylic acid and/or C₁₋₄ carboxylic ester group, wherein the heteroatom represents one or more of an oxygen atom, provided that R¹ is not a 2-hexylidenecyclopentyl group;
R² is a C₆₋₁₈ hydrocarbon group optionally comprising one or two oxygen atoms, provided that an ester functional group alpha to the oxy group is excluded; and
provided that R² is not a benzyl group or a cyclohexyl group or 2-hydroxy-1,2-diphenylethyl, or a 1-(tert-butoxy)-7,7-dimethylbicyclo[2.2.1]heptan-2-yl group and does not comprise an allylic functional group; and provided that 1-(heptyloxy)dec-1-ene, 1-(decyloxy)dec-1-ene, 1-(dodecyloxy)dodec-1-ene, (4-phenethoxybut-3-en-1-yl)benzene and 1-((3,7-dimethyloctyl)oxy)-3,7-dimethyloct-1-ene are excluded.

16. The method according to claim 2, wherein the compound of formula (I) is a C₂₀₋₃₆ compound.

17. A perfumed consumer product comprising a perfuming composition as defined in claim 11.
